(19) United States
(12) Patent Application Publication
(10) Pub. No.: US 2015/0316552 A1
(43) Pub. Date: Nov. 5, 2015

(54) Human Notch Receptor Mutations and Their Use

(71) Applicant: OncoMed Pharmaceuticals, Inc., Redwood City, CA (US)

(72) Inventors: Jennifer Anne Cain, Redwood City, CA (US); Min Wang, Burlingame, CA (US); Ann M. Kapoun, Mountain View, CA (US); Timothy C. Hoey, Hillsborough, CA (US)

(21) Appl. No.: 14/358,331

(22) PCT Filed: Nov. 14, 2012
(86) PCT No.: PCT/US2012/064969

§ 371 Date: Jun. 3, 2015

Related U.S. Application Data
(60) Provisional application No. 61/560,627, filed on Nov. 16, 2011.
(60) Provisional application No. 61/704,006, filed on Sep. 21, 2012.

(51) Int. Cl. G01N 33/574; C12Q 1/68; A61K 39/395; C07K 16/28; A61K 38/55; A61K 45/06; G01N 33/50; C07K 14/705; A61K 31/551

(52) U.S. Cl. CPC G01N 33/57415; C07K 14/705; G01N 33/57492; C12Q 1/6886; A61K 39/39558; A61K 31/551; A61K 38/55; A61K 45/06; G01N 33/5011; C07K 16/28; C12Q 2600/106; C12Q 2600/156; G01N 2800/52; C07K 2317/565; C07K 2317/76; G01N 2500/10

(57) ABSTRACT

The present invention provides the identification and characterization of NOTCH mutations associated with enhanced receptor signaling. The present invention provides methods and kits for using the same. The present invention further provides methods of treating cancer in a patient having a solid tumor, wherein the solid tumor cells comprise an elevated level of NOTCH ICD.

### FIELD OF INVENTION

The present invention relates to the field of oncology and provides the identification and characterization of NOTCH receptors that comprise mutations that result in increased receptor signaling. The present invention also provides methods and kits for using the same. The present invention further provides methods of treating cancer in a patient having a solid tumor, wherein the solid tumor cells comprise an elevated level of NOTCH ICD.

### BACKGROUND

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for over half of all new cases (Jemal et al., Cancer J. Clin. 53:5-26 (2003)). Increasingly, treatment of cancer has moved from the use of systemically acting cytotoxic drugs to include more targeted therapies that hone in on the mechanisms that allow unregulated cell growth and survival.

NOTCH receptors 1-4 are transmembrane receptor proteins that signal through a pathway that relies on regulated proteolysis. Following ligand-binding, the receptor is sequentially: i) cleaved extracellularly by metalloproteases of the ADAM family (Brou et al., Mol. Cell. 5:207-216 (2000); Mumm et al. Mol Cell 5:197-206 (2000)); ii) mono-ubiquitinated on a lysine residue lying just internal to the transmembrane domain (Gupta-Rossi et al., J. Cell Biol. 166:73-83 (2004)); iii) endocytosed (Gupta-Rossi, 2004), and iv) proteolytically cleaved by a gamma-secretase enzyme (De Strooper et al., Nature 398:518-522 (1999)). This final step in the activation process permits the intracellular portion of a NOTCH receptor to translocate to the cell nucleus where it interacts with transcription factors to alter gene activity. NOTCH receptor signaling plays an important role in the differentiation and proliferation of cells and in controlling apoptosis, three processes that are important with respect to neoplastic transformation.

The regulatory extracellular domain of NOTCH proteins consists largely of tandemly arranged EGF-like repeats that are required for ligand binding (Artavanis-Tsakonas et al. Science, 268:225-232 (1995)). Carboxy-terminal to the EGF-like repeats are an additional three cysteine-rich repeats, designated the LIN12/NOTCH repeats (LNR). Downstream of the LNR lies the proteolytic cleavage sequence (RXRR) that is recognized by a furin-like convertase. For NOTCH1, cleavage at this site yields a 180 kilodalton extracellular peptide and a 120 kilodalton intracellular peptide that are held together to generate a heterodimeric receptor at the cell surface (Blaumueller et al. Cell 90:281-91 (1997)).

The intracellular domain of NOTCH (NOTCH.ICD) rescues loss-of-function NOTCH phenotypes indicating that this form of NOTCH signals constitutively (Fortini, M. E., and S. Artavanis-Tsakonas. Cell 75:1245-7 (1993)). The cytoplasmic domain of NOTCH contains three identifiable domains: the RAM domain, the ankyrin repeat domain and the carboxy-terminal PEST domain. Upon ligand-activation NOTCH undergoes two additional proteolytic cleavages which results in the release of the cytoplasmic domain (Weinmaster, G. Curr Opin Genet Dev. 8:436-42 (1998)). This NOTCH peptide translocates to the nucleus and interacts with transcriptional repressors known as CSL (CBF, Su (H), Lag-2) and converts it to a transcriptional activator. The CSL/NOTCH interaction is dependent on the presence of the RAM domain of NOTCH; while, transcriptional activity also requires the presence of the ankyrin repeats (Hsieh, J. J. et al. J. Virol. 71:1938-45 (1997)). Both in vivo and in vitro studies indicate that the HES and Hey genes are the direct targets of NOTCH/CSL-dependent signaling (Nakagawa et al. Proc Natl Acad Sci USA 97:13655-13660 (2000)). The HES and HEY genes are bHLH transcriptional repressor that bind DNA at N-boxes. NOTCH has also been proposed to signal by a CSL-independent pathway. In fact, expression of just the ankyrin repeat domain is necessary and sufficient for some forms of NOTCH signaling (Lieber et al. Genes Dev. 7:1949-1965 (1993)). Finally, the PEST domain has been implicated in protein turnover by a SEL-10/ubiquitin-dependent pathway (Greenwald, I. Current Opinion in Genetics & Development 4:556-62 (1994)).

It has been previously shown that a (7;9) translocation creates a NOTCH-T cell receptor β fusion gene that encodes amino-terminally-deleted, constitutively active NOTCH-1 polypeptides similar to the ICD (Ellisen et al., Cell 66:649-661 (1991); Aster et al., Cold Spring Harb. Symp. Quant. Biol. 59:125-136 (1994)) and these truncated and constitutively active forms of NOTCH-1 induce T-cell acute lymphoblastic leukemia (T-ALL) in mouse models (Aster et al., Mol. Cell. Biol. 20:7505-7515 (2000)). In fact, activating mutations of NOTCH-1 have been reported in more than 50% of human T-ALLs (Weng et al., Science 306:269-271 (2004)). Leukemia-associated mutations within HD domain of NOTCH-1 have been shown to render the receptor sensitive to ligand-independent proteolytic activation (Malecki et al., Mol. Cell. Biol. 26:4642-4651 (2006)), while mutations in the PEST domain lead to reduced CDC4/FBW7-mediated degradation and stabilization of the intracellular cleaved form of NOTCH-1 (Pancewicz et al., Proc. Natl. Acad. Sci. USA 107:16619-16624 (2010)).

Although activating mutations in NOTCH-3, and NOTCH-4 have not been identified in human cancer, it is known that abnormal increases in the function of these NOTCH receptors in other mammals can cause T-ALL (NOTCH-2 and -3, Bellavia et al., Embo J. 19:3337-3348 (2000); Rohn J. Virol. 70:8071-8080 (1996); Weng et al., Mol. Cell. Biol. 23:655-664)), B-cell lymphoma (NOTCH-2, Lee et al., Cancer Sci. 100:920-926 (2009)), and breast cancer (NOTCH-4, Callahan and Rafat, J. Mammary Gland Biol Neoplasia 6:23-36 (2001)).

Identification of novel mutations in human solid tumors are useful diagnostically in helping to identify the presence of cancer and in identifying cancer cells that respond to inhibitors of NOTCH signaling, thereby making it possible to direct rational cancer treatment with NOTCH signaling pathway inhibitors.

### SUMMARY OF THE INVENTION

The invention provides for the identification of groups of cancer cells that depend upon abnormal NOTCH activity to maintain uncontrolled growth. In certain embodiments, the invention demonstrates that these cells comprise mutated NOTCH receptors and these mutations can be used diagnostically to identify cancer cells likely to respond to NOTCH inhibitor therapy or other factors that diminish NOTCH activity.

Thus, in one embodiment, the invention is directed to an isolated polynucleotide encoding a mutant human NOTCH1 receptor, wherein the polynucleotide comprises a deletion at nucleotide 7279 of the human NOTCH1 gene. In another embodiment, the mutation is a guanine (G) deletion at nucleotide 7279.

The invention is also directed to an isolated polynucleotide encoding a mutant human NOTCH3 receptor, wherein the polynucleotide comprises an insertion at position 6622 of the human NOTCH3 gene. In another embodiment, the mutation is a cytosine (C) insertion at position 6622.

The invention is also directed to an isolated polynucleotide encoding a mutant human NOTCH3 receptor, wherein the polynucleotide comprises an insertion at position 6096 of the human NOTCH3 gene. In another embodiment, the mutation is a cytosine (C) insertion at position 6096.

The invention is also directed to an isolated polynucleotide encoding a mutant human NOTCH1 receptor, wherein the polynucleotide comprises a substitution at position 6733 of the human NOTCH1 gene. In another embodiment, the mutant polynucleotide comprises an adenine (A) or cytosine (C) at position 6733. In another embodiment, the mutation is a guanine (G) to adenine (A) or a guanine (G) to cytosine (C) substitution at position 6733.

The invention is also directed to an isolated polynucleotide encoding a mutant human NOTCH1 receptor, wherein the polynucleotide comprises a substitution at position 6788 of the human NOTCH1 gene. In another embodiment, the mutation is a substitution to adenine (A) at position 6788. In another embodiment, the mutation is a guanine (G) to adenine (A) substitution at position 6788.

In one embodiment, the invention is directed to an isolated polypeptide encoded by the mutated NOTCH receptors described herein. In a further embodiment, the invention is directed to a vector comprising the polynucleotides of the invention. In one embodiment, the invention is directed to a host cell transformed with the vector.

In one embodiment, the invention is directed to a method of identifying solid tumor cells that exhibit increased NOTCH receptor signaling, comprising identifying whether the cells contain a mutation in the proline, glutamate, serine, threonine-rich (PEST) domain or in the TAD domain of human NOTCH1, wherein the solid tumor cells are selected from the group consisting of: a glioma, a gastrointestinal tumor, a renal tumor, an ovarian tumor, a liver tumor, a colorectal tumor, an endometrial tumor, kidney tumor, prostate tumor, thyroid tumor, neuroblastoma, pancreatic tumor, glioblastoma multiforme, cervical tumor, stomach tumor, bladder tumor, hepatoma, breast tumor, colon tumor, melanoma, biliary tract tumor, and head and neck tumor.

In another embodiment, the invention is directed to a method of identifying solid tumor cells that exhibit increased NOTCH receptor signaling, comprising identifying whether the cells contain a mutation of the PEST domain or in the TAD domain of human NOTCH2, wherein the solid tumor cells are selected from the group consisting of: a lung tumor, glioma, a gastrointestinal tumor, a renal tumor, an ovarian tumor, a liver tumor, a colorectal tumor, an endometrial tumor, kidney tumor, prostate tumor, thyroid tumor, neuroblastoma, pancreatic tumor, glioblastoma multiforme, cervical tumor, stomach tumor, bladder tumor, hepatoma, colon tumor, melanoma, biliary tract tumor, and head and neck tumor.

In another embodiment, the invention is directed to a method of identifying solid tumor cells that exhibit increased NOTCH receptor signaling, comprising identifying whether the cells contain a mutation of the PEST domain or in the TAD domain of NOTCH3, wherein the solid tumor cells are selected from the group consisting of: a lung tumor, a glioma, a gastrointestinal tumor, a renal tumor, an ovarian tumor, a liver tumor, a colorectal tumor, an endometrial tumor, kidney tumor, prostate tumor, thyroid tumor, neuroblastoma, pancreatic tumor, glioblastoma multiforme, cervical tumor, stomach tumor, bladder tumor, hepatoma, breast tumor, colon tumor, melanoma, biliary tract tumor, and head and neck tumor.

In another embodiment, the invention is directed to a method of identifying solid tumor cells that exhibit increased NOTCH receptor signaling, comprising identifying whether the cells contain a mutation of the PEST domain or in the TAD domain of NOTCH4, wherein the solid tumor cells are selected from the group consisting of: a lung tumor, glioma, a gastrointestinal tumor, a renal tumor, an ovarian tumor, a liver tumor, a colorectal tumor, an endometrial tumor, kidney tumor, prostate tumor, thyroid tumor, neuroblastoma, pancreatic tumor, glioblastoma multiforme, cervical tumor, stomach tumor, bladder tumor, hepatoma, breast tumor, colon tumor, melanoma, biliary tract tumor, and head and neck tumor.

In one embodiment, the mutation is a missense, nonsense, or frameshift mutation. In another embodiment, the mutation is a frameshift or nonsense mutation of the PEST domain. In another embodiment, the mutation is a deletion at nucleotide 7279 of the human NOTCH1 gene. In a further embodiment, the mutation is a guanine (G) deletion at nucleotide 7279 (B40 mutation) of the human NOTCH1 gene. In yet another embodiment, the mutation is an insertion at position 6622 of the human NOTCH3 gene. In a further embodiment, the mutation is a cytosine (C) insertion at position 6622 of the human NOTCH3 gene (B37 mutation). In yet another embodiment, the mutation is an insertion at position 6096 of the human NOTCH3 gene. In a further embodiment, the mutation is a cytosine (C) insertion at position 6096 of the human NOTCH3 gene (C31 mutation). In yet another embodiment, the mutation is a substitution at position 6733 of the human NOTCH1 gene. In a further embodiment, the mutation is a substitution to adenine (A) or cytosine (C) at position 6733 of the human NOTCH1 gene. In a further embodiment, the mutation is a guanine (G) to adenine (A) or a guanine (G) to cytosine (C) substitution at position 6733 of the human NOTCH1 gene (Lung_{—}01246 mutation). In yet another embodiment, the mutation is a substitution at position 6788 of the human NOTCH1 gene. In a further embodiment, the mutation is a substitution to adenine (A) at position 6788 of the human NOTCH1 gene. In a further embodiment, the mutation is a guanine (G) to adenine (A) substitution at position 6788 of the human NOTCH1 gene (Breast_H12932T mutation).

In one embodiment, the presence of the mutation is determined using an anti-NOTCH antibody or a nucleic acid probe that hybridizes to the mutant NOTCH polynucleotide under stringent conditions. In another embodiment, the antibody or nucleic acid probe is detectably labeled. In another embodiment, the label is selected from the group consisting of immunofluorescent label, chemiluminescent label, phosphorescent label, enzyme label, radiolabel, avidin/biotin, colloidal gold particles, colored particles and magnetic particles. In another embodiment, the presence of the mutations is determined by radioimmunoassay, Western blot assay, immunofluorescent assay, enzyme immunoassay, immunoprecipitation assay, chemiluminescent assay, immunohistochemical assay, dot blot assay, slot blot assay or flow cytometry assay. In yet another embodiment, the presence of the mutations is determined by RT-PCR. In a further embodiment, the presence of the mutations is determined by microarray. In another embodiment, the presence of the mutations is determined by nucleic acid sequencing.

The invention is also directed to a method for stratifying a cancer patient population for treatment with a NOTCH inhibitor comprising: (a) determining whether tumor cells from the patients contain an activating mutation of the PEST domain of a human NOTCH receptor, and (b) stratifying the patient population based on the presence or absence of the mutation.

The invention is also directed to a method for selecting a patient for treatment with a NOTCH inhibitor comprising: (a) determining whether tumor cells from the patient contain an activating mutation of the PEST domain of a human NOTCH receptor, and (b) selecting those patients whose tumor cells contain the mutation.

The invention is also directed to a method for determining whether a patient diagnosed with cancer is likely to respond to a NOTCH inhibitor-based therapy comprising the steps of determining whether tumor cells from the patient contain an activating mutation of the PEST domain of a human NOTCH receptor, wherein the presence of the mutation indicates that the patent is likely to respond to therapy.

The invention is also directed to a method for determining whether a patient diagnosed with cancer should be administered a NOTCH inhibitor, comprising determining whether tumor cells from the patient contain an activating mutation of the PEST domain of a human NOTCH receptor, wherein the presence of the mutation is predictive of the patient having a favorable response to treatment with a NOTCH inhibitor.

The invention is also directed to a method to determine whether a patient diagnosed with cancer should continue treatment with a NOTCH inhibitor, comprising determining whether tumor cells from the patient contain an activating mutation of the PEST domain of a human NOTCH receptor, wherein the presence of either mutation indicates that the patient is likely to respond to therapy, wherein the presence of the mutation is predictive of the patient having a favorable response to treatment with the NOTCH inhibitor.

The invention is also directed to a method for determining the therapeutic efficacy of a NOTCH inhibitor for treating cancer in a patient comprising determining whether tumor cells from the patient contain an activating mutation of the PEST domain of a human NOTCH receptor, wherein the presence of the mutation is indicative of the therapeutic efficacy of the NOTCH inhibitor.

In one embodiment, the patient is a human. In one embodiment, the mutation increases NOTCH signaling. In another embodiment, at least about 0.1%, at least about 1%, at least about 2%, or at least about 5%, of the tumor cells from the patient comprise the mutation. In another embodiment, the NOTCH receptor is NOTCH1 or NOTCH3. In another embodiment, the mutation is a missense, nonsense, or frameshift mutation. In a further embodiment, the mutation is a frameshift or nonsense mutation of the PEST domain. In another embodiment, the mutation is a deletion at nucleotide 7279 of the human NOTCH1 gene. In another embodiment, the mutation is a guanine (G) deletion at nucleotide 7279 (B40 mutation) of the human NOTCH1 gene. In another embodiment, the mutation is an insertion at position 6622 of the human NOTCH3 gene. In another embodiment, the mutation is a cytosine (C) insertion at position 6622 of the human NOTCH3 gene (B37 mutation). In another embodiment, the mutation is an insertion at position 6096 of the human NOTCH3 gene. In a further embodiment, the mutation is a cytosine (C) insertion at position 6096 of the human NOTCH3 gene (C31 mutation). In another embodiment, the mutation is a substitution at position 6733 of the human NOTCH1 gene. In a further embodiment, the mutation is a substitution to adenine (A) or cytosine (C) at position 6733 of the human NOTCH1 gene. In a further embodiment, the mutation is a guanine (G) to adenine (A) or a guanine (G) to cytosine (C) substitution at position 6733 of the human NOTCH1 gene (Lung_{—}01246 mutation). In another embodiment, the mutation is a substitution at position 6788 of the human NOTCH1 gene. In a further embodiment, the mutation is a substitution to adenine (A) at position 6788 of the human NOTCH1 gene. In a further embodiment, the mutation is a guanine (G) to adenine (A) substitution at position 6788 of the human NOTCH1 gene (Breast_H12932T mutation).

In one embodiment, the methods further comprise obtaining a body sample from the patient. In another embodiment, the sample is whole blood, plasma, serum, or tissue. In another embodiment, the cancer is selected from the group consisting of: lung, gastrointestinal, renal, ovarian, liver, colorectal, endometrial, kidney, prostate, thyroid, neuroblastoma, pancreatic, glioblastoma multiforme, cervical, stomach, bladder, breast, colon, melanoma, biliary tract, and head and neck cancer.

In one embodiment, the presence of the mutations is determined using an anti-NOTCH antibody or a nucleic acid probe that hybridizes to the mutant NOTCH polynucleotide under stringent conditions. In another embodiment, the antibody or nucleic acid probe is detectably labeled. In another embodiment, the label is selected from the group consisting of immunofluorescent label, chemiluminescent label, phosphorescent label, enzyme label, radiolabel, avidin/biotin, colloidal gold particles, colored particles and magnetic particles. In another embodiment, the presence of the mutations is determined by radioimmunoassay, Western blot assay, immunofluorescent assay, enzyme immunoassay, immunoprecipitation assay, chemiluminescent assay, immunohistochemical assay, dot blot assay or slot blot assay. In another embodiment, the presence of the mutations is determined by RT-PCR. In another embodiment, the presence of the mutations is determined by microarray. In another embodiment, the presence of the mutations is determined by nucleic acid sequencing.

In one embodiment, the methods further comprise administering a NOTCH inhibitor to the patient. In another embodiment, the NOTCH inhibitor is a gamma-secretase inhibitor or an anti-NOTCH antibody. In another embodiment, the gamma-secretase inhibitor is selected from the group consisting of: III-31-C; N—[N-(3,5-difluorophenacetyl)-L-alanyl]S-phenylglycine t-butyl ester) (DAPT); compound E; D-helical peptide 294; isocoumarins; BOC-Lys(Cbz)Ile-Leu-epoxide; and (Z-LL)2-ketone. In another embodiment, the anti-NOTCH antibody is an anti-NOTCH1 antibody. In another embodiment, the anti-NOTCH1 antibody blocks ligand binding to the NOTCH1 receptor. In another embodiment, the anti-NOTCH1 antibody blocks cleavage of the NOTCH1 receptor. In another embodiment, the anti-NOTCH1 antibody comprises the heavy chain variable region comprising CDR amino acid sequences CDR1 (SEQ ID NO:5), CDR2 (SEQ ID NO:6), and CDR3 (SEQ ID NO:7), and the light chain variable region comprising CDR amino acid sequences CDR1 (SEQ ID NO: 8); CDR2 (SEQ ID NO: 9); and CDR3 (SEQ ID NO: 10). In another embodiment, the anti-NOTCH antibody is an anti-NOTCH3 antibody. In another embodiment, the anti-NOTCH3 antibody blocks ligand binding to the NOTCH3 receptor. In another embodiment, the anti-NOTCH3 antibody blocks cleavage of the NOTCH3 receptor. In another embodiment, the anti-NOTCH3 antibody comprises the heavy chain variable region comprising CDR amino acid sequences CDR1 (SEQ ID NO:23), CDR2 (SEQ ID NO:24), and CDR3 (SEQ ID NO:25), and the light chain variable region comprising CDR amino acid sequences CDR1 (SEQ ID NO:26); CDR2 (SEQ ID NO: 27); and CDR3 (SEQ ID NO: 28).

The invention is also directed to a method of treating cancer in a patient having a solid tumor comprising administering to the patient a therapeutically effective amount of a NOTCH1 inhibitor, wherein at least one of the solid tumor cells in the patient comprises an activating mutation in the human NOTCH1 gene. The invention is also directed to a method of treating breast cancer in a patient having a breast tumor comprising administering to the patient a therapeutically effective amount of a NOTCH1 inhibitor, wherein at least one of the breast tumor cells in the patient comprises an activating mutation in the human NOTCH1 gene. In one embodiment, the activating mutation is of the PEST domain. In another embodiment, the mutation increases NOTCH signaling. In another embodiment, the mutation comprises a guanine deletion at nucleotide 7279 of the human NOTCH1 gene. In a further embodiment, the mutation is a guanine (G) to adenine (A) or a guanine (G) to cytosine (C) substitution at position 6733 of the human NOTCH1 gene (Lung_{—}01246 mutation). In another embodiment, the mutation is a guanine (G) to adenine (A) substitution at position 6788 of the human NOTCH1 gene (Breast_H12932T mutation).

The invention is also directed to a method of treating cancer in a patient having a solid tumor comprising administering to the patient a therapeutically effective amount of a NOTCH3 inhibitor, wherein at least one of the solid tumor cells in the patient comprises an activating mutation in the human NOTCH3 gene. The invention is also directed to a method of treating breast cancer in a patient having a breast tumor comprising administering to the patient a therapeutically effective amount of a NOTCH3 inhibitor, wherein at least one of the breast tumor cells in the patient comprises an activating mutation in the human NOTCH3 gene. In one embodiment, the activating mutation is of the PEST domain. In another embodiment, the mutation increases NOTCH signaling. In another embodiment, the mutation comprises a cytosine insertion at position 6622 of human NOTCH3 gene. In a further embodiment, the mutation is a cytosine (C) insertion at position 6096 of the human NOTCH3 gene (C31 mutation).

In one embodiment, the patients are humans. In one embodiment, at least about 0.1%, at least about 1%, at least about 2%, or at least about 5%, of the tumor cells from the patient comprise the mutation. In another embodiment, the NOTCH1 inhibitor is a gamma-secretase inhibitor or an anti-NOTCH1 antibody. In another embodiment, the gamma-secretase inhibitor is selected from the group consisting of: III-31-C; N—[N-(3,5-difluorophenacetyl)-L-alanyl]S-phenylglycine t-butyl ester) (DAPT); compound E; D-helical peptide 294; isocoumarins; BOC-Lys(Cbz)Ile-Leu-epoxide; and (Z-LL)2-ketone. In another embodiment, the anti-NOTCH1 antibody blocks ligand binding to the NOTCH1 receptor. In another embodiment, the anti-NOTCH1 antibody blocks cleavage of the NOTCH1 receptor.

In one embodiment, the anti-NOTCH1 antibody comprises a heavy chain variable region comprising CDR amino acid sequences CDR1 (SEQ ID NO:5), CDR2 (SEQ ID NO: 6), and CDR3 (SEQ ID NO: 7), and a light chain variable region comprising CDR amino acid sequences CDR1 (SEQ ID NO: 8); CDR2 (SEQ ID NO: 9); and CDR3 (SEQ ID NO: 10). In one embodiment, the anti-NOTCH1 antibody comprises the heavy chain variable region sequence of SEQ ID NO:14 and the light chain variable region sequence of SEQ ID NO: 18. In another embodiment the anti-NOTCH1 antibody is OMP-52M51.

In one embodiment, the NOTCH3 inhibitor is selected a gamma-secretase inhibitor or an anti-NOTCH3 antibody. In another embodiment, the gamma-secretase inhibitor is selected from the group consisting of: III-31-C; N—[N-(3,5-difluorophenacetyl)-L-alanyl]S-phenylglycine t-butyl ester) (DAPT); compound E; D-helical peptide 294; isocoumarins; BOC-Lys(Cbz)Ile-Leu-epoxide; and (Z-LL)2-ketone. In another embodiment, the anti-NOTCH3 antibody blocks ligand binding to the NOTCH3 receptor. In another embodiment, the anti-NOTCH3 antibody blocks cleavage of the NOTCH3 receptor. In another embodiment, the anti-NOTCH3 antibody comprises the heavy chain variable region comprising CDR amino acid sequences CDR1 (SEQ ID NO: 23), CDR2 (SEQ ID NO: 24), and CDR3 (SEQ ID NO: 25), and the light chain variable region comprising CDR amino acid sequences CDR1 (SEQ ID NO: 26); CDR2 (SEQ ID NO: 27); and CDR3 (SEQ ID NO: 28). (59R5)

In one embodiment, the patient comprises triple negative breast cancer cells.

In one embodiment, the method further comprises administering a second therapeutic agent.

In another embodiment, the patient previously failed a cancer therapy. In another embodiment, the patient has chemotherapy resistant breast cancer.

The invention is also directed to a method of identifying a test compound that inhibits abnormal cell growth induced by the presence of a mutated NOTCH receptor, comprising: (a) incubating the test compound with cells that express the mutant NOTCH receptor, the incubation performed in the presence of gamma-secretase, (b) comparing the amount of receptor activity in step (a) with the activity of an incubation performed in the absence of the test compound, wherein the test compound inhibits cell growth if the activity observed in the presence of the test compound is less than the activity observed in the absence of the test compound.

The invention is also directed to a kit comprising at least one reagent for specifically detecting a mutated NOTCH receptor of the invention. In one embodiment, the reagent is an antibody or nucleic acid probe that binds a mutated NOTCH receptor of the invention.

Additional objects and advantages of the invention will be set forth in part in the description which follows, and in part will be obvious from the description, or can be learned by routine practice of the invention. The objects and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The invention further provides for the identification of groups of cancer cells that depend upon abnormal NOTCH activity to maintain uncontrolled growth. In certain embodiments, the invention demonstrates that these cells comprise a level of NOTCH ICD above the level of a control sample or above another reference level, and that the level of NOTCH ICD in tumor cells can be used diagnostically to identify cancer cells likely to respond to NOTCH inhibitor therapy or other factors that diminish NOTCH activity.

Thus, in one embodiment, the invention is directed to a method for stratifying a cancer patient population for treatment with a NOTCH inhibitor comprising: (a) determining the level of NOTCH ICD in tumor cells from said patients, and (b) stratifying the patient population based on the level of NOTCH ICD in tumor cells.

In another embodiment, the invention is directed to a method for selecting a patient for treatment with a NOTCH inhibitor comprising: (a) determining the level of NOTCH ICD in tumor cells from the patient, and (b) selecting the patient whose tumor cells comprise a level of NOTCH ICD above the level of a control sample or above a reference level. The invention also provides a method for selecting a cancer patient for treatment with a NOTCH1 inhibitor (e.g., OMP-52M51) comprising: (a) determining the level of NOTCH1 ICD in solid tumor cells from the patient in an immunohistochemical assay with an anti-NOTCH1 ICD antibody, and (b) selecting for treatment the patient whose solid tumor cells receive an H-score of about 30 or more in the assay (or, alternatively, about 100 or more in the assay).

In another embodiment, the invention is directed to a method for determining whether a patient diagnosed with cancer is likely to respond to a NOTCH inhibitor-based therapy comprising the step of determining the level of NOTCH ICD in tumor cells from said patient, wherein a level of NOTCH ICD above a reference level or above the level of a control sample indicates that the patent is likely to respond to therapy.

In another embodiment, the invention is directed to a method for determining whether a patient diagnosed with cancer should be administered a NOTCH inhibitor, comprising determining the level of NOTCH ICD in tumor cells from said patient, wherein a level of NOTCH ICD above a reference level or above the level of a control sample is predictive of said patient having a favorable response to treatment with a NOTCH inhibitor.

In another embodiment, the invention is directed to a method to determine whether a patient diagnosed with cancer should continue treatment with a NOTCH inhibitor, comprising determining the level of NOTCH ICD in tumor cells from said patient, wherein a level of NOTCH ICD above a reference level or above the level of a control sample indicates that the patient is likely to respond to therapy with said NOTCH inhibitor.

In another embodiment, the invention is directed to a method for determining the therapeutic efficacy of a NOTCH inhibitor for treating cancer in a patient comprising determining the level of NOTCH ICD in tumor cells from said patient, wherein a level of NOTCH ICD above a reference level or above the level of a control sample is indicative of the therapeutic efficacy of said NOTCH inhibitor.

In some embodiments, the NOTCH ICD is NOTCH1 ICD. In alternative embodiments, the NOTCH ICD is NOTCH3 ICD. In a further embodiment, the level of NOTCH ICD is the level of NOTCH ICD in the nucleus of tumor cells.

In an additional embodiment, the method further comprises obtaining a body sample from said patient. In a further embodiment, the sample is whole blood, plasma, serum, or tissue.

In an additional embodiment, the cancer is selected from the group consisting of: lung cancer, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, breast cancer, colon cancer, melanoma, biliary tract cancer, and head and neck cancer. In a further embodiment, the cancer is breast cancer. In a further embodiment, the cancer is small cell cancer, small cell lung cancer, gastric cancer, esophageal cancer, hepatocellular carcinoma, or cholangiocarcinoma.

In an additional embodiment, the NOTCH ICD level is determined using an agent that specifically binds to NOTCH ICD. In a further embodiment, the agent is an anti-NOTCH ICD antibody. In a further embodiment, the anti-NOTCH ICD antibody is a polyclonal antibody or a monoclonal antibody.

In an additional embodiment, the agent is detectably labeled. In a further embodiment, the label is selected from the group consisting of immunofluorescent label, chemiluminescent label, phosphorescent label, enzyme label, radiolabel, avidin/biotin, colloidal gold particles, colored particles and magnetic particles.

In an additional embodiment, the level of NOTCH ICD is determined by a radioimmunoassay, immunofluorescent assay, enzyme immunoassay, chemiluminescent assay, or immunohistochemical assay. In a further embodiment, the level of NOTCH ICD (and/or a reference level to which that level is compared) is characterized by an H-score.

In an additional embodiment, the method further comprises administering a NOTCH inhibitor to said patient.

In another embodiment, the invention is directed to a method of treating cancer in a patient having a solid tumor comprising: (a) determining the level of NOTCH ICD in the solid tumor cells; and (b) administering to said patient a therapeutically effective amount of a NOTCH inhibitor. In certain embodiments, the NOTCH ICD is NOTCH1 ICD, the NOTCH inhibitor is a NOTCH1 inhibitor (e.g., OMP-52M51), and the solid tumor cells are determined to have an H-score of about 30 or more in an immunohistochemical assay with an anti-NOTCH1 ICD antibody. In certain alternative embodiments, the solid tumor cells are determined of have an H-score of about 100 or more in the assay.

In an additional embodiment, the NOTCH inhibitor is a gamma-secretase inhibitor or an anti-NOTCH antibody. In a further embodiment, the gamma-secretase inhibitor is selected from the group consisting of: III-31-C; N—[N-(3,5-difluorophenacetyl)-L-alanyl]S-phenylglycine t-butyl ester) (DAFT); compound E; D-helical peptide 294; isocoumarins; BOC-Lys(Cbz)Ile-Leu-epoxide; and (Z-LL)2-ketone.

In an additional embodiment, the anti-NOTCH antibody is an anti-NOTCH1 antibody. In a further embodiment, the anti-NOTCH1 antibody blocks ligand binding to the NOTCH1 receptor. In a further embodiment, the anti-NOTCH1 antibody blocks cleavage of the NOTCH1 receptor. In a further embodiment, the anti-NOTCH1 antibody comprises a heavy chain variable region comprising CDR amino acid sequences CDR1 (SEQ ID NO:5), CDR2 (SEQ ID NO:6), and CDR3 (SEQ ID NO:7), and a light chain variable region comprising CDR amino acid sequences CDR1 (SEQ ID NO: 8); CDR2 (SEQ ID NO: 9); and CDR3 (SEQ ID NO: 10). In one embodiment, the anti-NOTCH1 antibody comprises the heavy chain variable region sequence of SEQ ID NO:14 and the light chain variable region sequence of SEQ ID NO: 18. In another embodiment the anti-NOTCH1 antibody is OMP-52M51.

In an additional embodiment, the anti-NOTCH antibody is an anti-NOTCH3 antibody. In a further embodiment, the anti-NOTCH3 antibody blocks ligand binding to the NOTCH3 receptor. In a further embodiment, the anti-NOTCH3 antibody blocks cleavage of the NOTCH3 receptor. In a further embodiment, the anti-NOTCH3 antibody comprises a heavy chain variable region comprising CDR amino acid sequences CDR1 (SEQ ID NO:23), CDR2 (SEQ ID NO:24), and CDR3 (SEQ ID NO:25), and a light chain variable region comprising CDR amino acid sequences CDR1 (SEQ ID NO: 26); CDR2 (SEQ ID NO: 27); and CDR3 (SEQ ID NO: 28). In one embodiment, the anti-NOTCH3 antibody comprises the heavy chain variable region sequence of SEQ ID NO:30 and the light chain variable region sequence of SEQ ID NO: 32. In one embodiment the anti-NOTCH3 antibody is 59R5.

In an additional embodiment, the patient is a human.

In another embodiment, the invention is directed to a method of treating cancer in a patient having a solid tumor comprising administering to said patient a therapeutically effective amount of a NOTCH1 inhibitor, wherein the solid tumor cells in said patient (a) comprise NOTCH1 ICD at a level above a reference level or above the level found in a control sample or (b) are characterized by an H-score of about 30 or more (or, alternatively, about 100 or more) in an immunohistochemical assay with an anti-NOTCH1 ICD antibody.

In an additional embodiment, the NOTCH1 inhibitor is a gamma-secretase inhibitor or an anti-NOTCH1 antibody. In a further embodiment, the gamma-secretase inhibitor is selected from the group consisting of: III-31-C; N—[N-(3,5-difluorophenacetyl)-L-alanyl]S-phenylglycine t-butyl ester) (DAFT); compound E; D-helical peptide 294; isocoumarins; BOC-Lys(Cbz)Ile-Leu-epoxide; and (Z-LL)2-ketone.

In an additional embodiment, the anti-NOTCH1 antibody blocks ligand binding to the NOTCH1 receptor. In a further embodiment, the anti-NOTCH1 antibody blocks cleavage of the NOTCH1 receptor. In a further embodiment, the anti-NOTCH1 antibody comprises the heavy chain variable region comprising CDR amino acid sequences CDR1 (SEQ ID NO:5), CDR2 (SEQ ID NO:6), and CDR3 (SEQ ID NO:7), and the light chain variable region comprising CDR amino acid sequences CDR1 (SEQ ID NO: 8); CDR2 (SEQ ID NO: 9); and CDR3 (SEQ ID NO:10).

In an additional embodiment, the patient is a human.

In an additional embodiment, the method further comprises administering a second therapeutic agent.

In an additional embodiment, the patient previously failed a cancer therapy. In a further embodiment, the patient comprises triple negative breast cancer cells. In a further embodiment, the patient has chemotherapy resistant breast cancer.

In an additional embodiment, the patient has small cell cancer, small cell lung cancer, gastric cancer, esophageal cancer, hepatocellular carcinoma, or cholangiocarcinoma.

In an additional embodiment, the NOTCH ICD level is determined using an agent that specifically binds to NOTCH ICD. In a further embodiment, the agent is an anti-NOTCH ICD antibody. In a further embodiment, the anti-NOTCH ICD antibody is a polyclonal antibody or a monoclonal antibody.

In an additional embodiment, the agent is detectably labeled. In a further embodiment, the label is selected from the group consisting of immunofluorescent label, chemiluminescent label, phosphorescent label, enzyme label, radiolabel, avidin/biotin, colloidal gold particles, colored particles and magnetic particles.

In an additional embodiment, the level of NOTCH ICD is determined by a radioimmunoassay, immunofluorescent assay, enzyme immunoassay, chemiluminescent assay, or immunohistochemical assay. In a further embodiment, the level of NOTCH ICD is characterized by an H-score.

### DESCRIPTION OF THE FIGURES

FIG. 1: Schematic representation of newly identified OMP-B40 and OMP-B37 NOTCH mutations. (A) The OMP-B40 mutation is characterized as a heterozygous deletion of guanine (G) at the nucleotide site 7279 of the human NOTCH1 gene. The deletion causes a reading frame shift of the PEST (G2427fs) domain of NOTCH1. (B) The OMP-B37 mutation is characterized as a homozygous insertion of cytosine (C) at the nucleotide site 6622 in the human NOTCH3 gene which causes a reading frame shift at amino acid position 2208 (P2208fs) of the PEST domain. The schematic representation of NOTCH was adopted from Weng et al., Science 306:269-271 (2004).

FIG. 2: NOTCH expression analysis in xenograft tumors containing the OMP-B40 or OMP-B37 mutations. (A) An anti-NOTCH1.ICD antibody reacts specifically against the cleaved NOTCH1 intracellular domain (NOTCH1.ICD) and detects a truncated cleaved NOTCH1 intracellular domain in the nuclear fraction of the OMP-B40 xenograft tumor. (B) An anti-NOTCH3.ICD antibody reacts specifically against the cleaved NOTCH3.ICD and detects a truncated cleaved NOTCH3.ICD in the nuclear fraction of the OMP-B37 xenograft tumor. (C) Truncated NOTCH1 ICD and the truncated NOTCH3 ICD are found predominately in the nuclear fractions of the OMP-B40 and OMP-B37 xenograft tumors. These two tumor lines carry mutations of the PEST domains of NOTCH1 and NOTCH3, respectively. Nuclear NOTCH1 ICD and NOTCH3 ICD are not detected in the OMP-C31 and OMP-OV38 xenograft tumors that carry wild-type NOTCH1, but OMP-C31 contains a 6172insC(het) [P2033fs] mutation in the NOTCH3 gene.

FIG. 3: Activity of a NOTCH1 antagonist in OMP-B40 breast tumors. (A) Dose dependent activity of OMP-52M51 humanized anti-NOTCH1 antibody in OMP-B40 tumors. (B) The anti-NOTCH1 antibody OMP-52M51, taxol, or a combination of OMP-52M51 and taxol were administered to OMP-B40 xenograft mice. OMP-52M51, either alone or in combination with taxol, greatly decreased tumor volume in xenografted animals. (C and D) OMP-52M51 anti-NOTCH1 antibody blocks NOTCH1.ICD accumulation in breast cancer model with a NOTCH1 activating mutation. OMP-52M51 anti-NOTCH1 used as single agent or in combination with taxol inhibits NOTCH1.ICD accumulation as detected by IHC assay.

FIG. 4: Tumorigenicity of anti-NOTCH1-treated OMP-B40 breast tumors. Tumor cells from control antibody treated tumors and OMP-52M51 humanized anti-NOTCH1 antibody treated tumors were injected into 10 mice each. Tumors were allowed to grow for 92 days without further treatment. OMP-B40 tumors grew in each of the 10 mice injected with the control antibody treated cells, whereas only 2 out of 10 mice injected with cells previously treated with OMP-52M51 resulted in detectable tumor growth at day 92, indicating that OMP-52M51 treatment reduced the subsequent tumorigenecity of the cells.

FIG. 5: Activity of a NOTCH2/3 antagonist in OMP-B37 breast tumors. The anti-NOTCH2/3 antibody 59R5 or a control antibody were administered to OMP-B37 xenograft mice. 59R5 greatly decreased tumor volume in xenografted animals compared to administration of a control antibody.

FIG. 6: Activity of anti-NOTCH2/3 antibody in OMP-B37 breast tumor. The anti-NOTCH2/3 antibody 59R5, taxol, or a combination of 59R5 and taxol were administered to OMP-B37 xenograft mice. (A) 59R5, either alone or in combination with taxol, greatly decreased tumor volume in xenografted animals. (B) 59R5 Anti-NOTCH2/3 antibody increases expression of E-cadherin indicating reversal of Epithelial Mesenchymal Transition (EMT).

FIG. 7: (A) Sanger chromatogram of NOTCH3 frameshift mutation in OMP-C31. (B) Sanger chromatogram of NOTCH1 missense mutation in Lung_{—}01246. (C) Sanger chromatogram of NOTCH1 missense mutation in Breast_H12932T. The schematic representation of NOTCH was adopted from Weng et al., Science 306:269-271 (2004).

FIG. 8. Nuclear localization of activated NOTCH 1-ICD by immunohistochemistry.

FIG. 9. (A) NOTCH1.ICD screening in solid tumors. Frequency of samples with H-scores ≧30 are shown. (B) N1.ICD IHC analysis of esophageal carcinoma samples. (C) NOTCH1.ICD screening in gastric cancer. Frequency of samples with H-scores ≧30 are shown.

FIG. 10. Activity of OMP-52M51 humanized anti-NOTCH1 antibody in OMP-LU61 small cell lung cancer.

FIG. 11. Activity of OMP-52M51 humanized anti-NOTCH1 antibody in OMP-C63 colon cancer.

FIG. 12. Activity of OMP-52M51 humanized anti-NOTCH1 antibody in combination with irinotecan in OMP-C11, OMP-C20 and OMP-C40 tumor cells.

FIG. 13. Luciferase Assay of NOTCH1 and NOTCH3 mutants. (A) PC3 cells were transiently transfected with DNA encoding NOTCH1 full length wild type (NOTCH1_WT) or NOTCH1_G2427fs mutant (OMP-B40) polypeptide. NOTCH signaling mediated luciferase activity was assessed in the absence of exogenous ligand. (B) PC3 cells and A549 cells were transiently transfected with DNA encoding NOTCH3 full length wild type (NOTCH3_WT), NOTCH3_P2033fs mutant (OMP-C31), or NOTCH3_P2208fs mutant (OMP-B37) polypeptide. NOTCH luciferase activity was assessed in the absence of exogenous ligand. (C) Dose response curves for JAG1 (1-500 ng/30 μL) in PC3 cells transiently transfected with DNA encoding NOTCH3_P2033fs (OMP-C31) polypeptide. (D) Dose response curves for JAG1 (1-500 ng/30 μL) in PC3 cells transiently transfected with NOTCH3_P2208fs (OMP-B37) encoding DNA. * =p-value <0.05 for NOTCH full length WTvs N3.mutant using t-test.

FIG. 14. A2G1 anti-NOTCH1 antibody treatment inhibits tumor growth and NOTCHI ICD accumulation in the OMP-B40 breast tumor model with less severe gastrointestinal toxicity than treatment with a gamma secretase inhibitor (GSI).

FIG. 15. Activity of the 59R5 anti-NOTCH2/3 antibody in OMP-C31 colon tumor.

FIG. 16. 52M51 anti-NOTCH1 antibody inhibits the activity of the G2427fs (OMP-B40) and R2328W NOTCH1_mutant polypeptide as measured in a luciferase reporter assay. FIGS. 16A and B show the Firefly luciferase to Renilla luciferase activity ratio observed in G2427fs (OMP-B40) NOTCH1_mutant polypeptide expressing PC3 cells after stimulation with DLL4 and JAG1, respectively, in the presence of increasing concentrations of 52M51 antibody. FIGS. 16C and D show the Firefly luciferase to Renilla luciferase activity ratio observed in R2328W NOTCH1_mutant polypeptide expressing PC3 cells after stimulation with DLL4 and JAG1, respectively, in the presence of increasing concentrations of 52M51 antibody. All of FIGS. 16A-D also includes data obtained with control PC3 cells that did not express a recombinant NOTCH1 polypeptide.

FIG. 17. Activity of NOTCH2/3 antagonist in OMP-B37 breast tumors. OMP-59R5 anti-NOTCH2/3 antibody blocks NOTCH3.ICD accumulation in breast cancer model with a NOTCH3 activating mutation.

### DESCRIPTION OF THE EMBODIMENTS

### I. Definitions

For purposes of the present invention, the following terms are defined below.

It is to be noted that the term “a” or “an” entity refers to one or more of that entity; for example, “a NOTCH receptor polypeptide” is understood to represent one or more polypeptides that comprise a NOTCH receptor amino acid. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis.

An “isolated” biological component (such as a nucleic acid molecule or protein) has been substantially separated or purified away from other biological components in the cell of the organism in which the component naturally occurs, i.e., other chromosomal and extrachromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

The term “polynucleotide,” when used in singular or plural, generally refers to any a polyribonucleotide or polydeoxyribonucleotide, which can be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides. Polynucleotides can be made by a variety of methods, including in vitro recombinant DNA-mediated techniques and by expression of DNAs in cells and organisms.

The term “naturally-occurring” or “wild-type” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is wild-type.

A “mutation” as used herein refers to variations that arise through somatic mutation, for instance those that are found only in disease cells, but not constitutionally, in a given subject. Examples of such somatically-acquired variations include the point mutations that frequently result in altered function of various genes that are involved in development of cancers. Types of mutations include base substitution point mutations (e.g., transitions or transversions), deletions, and insertions. Missense mutations are those that introduce a different amino acid into the sequence of the encoded protein; nonsense mutations are those that introduce a new stop codon. In the case of insertions or deletions, mutations can be in-frame (not changing the frame of the overall sequence) or frame shift mutations, which can result in the misreading of a large number of codons (and often leads to abnormal termination of the encoded product due to the presence of a stop codon in the alternative frame). The mutations of the invention can be either found on one allele (heterozygous) or on both alleles (homozygous) in a subject. Activating mutations that affect the function of domains of the NOTCH receptor, in particular the PEST domain (rich in proline, glutamate, serine, and threonine), can include mutations that eliminate some or all of the domain by truncation (e.g. a nonsense or frameshift mutation). Activating mutations can also include missense mutations in the domains themselves (e.g. within the PEST domain) that impede function of the domains.

An “activating mutation” is a somatic mutation that causes NOTCH to be constitutively active, hypersensitive to ligand stimulation, or aberrantly expressed. In some embodiments, the activating mutations result in increased NOTCH signaling. The amount of NOTCH signaling can be determined by methods known in the art. Briefly, the amount of signaling from a mutated NOTCH polypeptide is compared to the amount of NOTCH signaling from a wild-type NOTCH polypeptide. As used herein, “increased NOTCH signaling” or “enhanced NOTCH signaling” refers to a higher amount of NOTCH signaling in cells containing a mutated NOTCH polypeptide compared to NOTCH signaling in cells containing a wild-type NOTCH polypeptide. A variety of methods known in the art can be used to detect NOTCH signaling. Exemplary methods include, but are not limited to, NOTCH ICD western blotting or immunohistochemistry (IHC) (See, Wu et al., Nature, 464:1052-1059 (2010)). Generally, expression/signaling in normal tissue is the standard for comparison to determine signaling above normal levels. In some embodiments, cells from normal tissue adjacent to the cells of interest are used to determine signaling levels. In some NOTCH ICD assays, the level of NOTCH ICD in normal tissue is undetectable, thus any signal above background would be considered increased.

The term “operably linked” as used herein refers to the positioning of components such that they are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequences; in eukaryotes, generally, such control sequences include promoters and transcription termination sequences. The term “control sequences” is intended to include, at a minimum, all components whose presence is essential for expression and processing, and can also include additional components whose presence is advantageous, for example, leader sequences and fusion partner sequences.

The similarity between two nucleic acid sequences, or two amino acid sequences, is expressed in terms of the similarity between the sequences, otherwise referred to as “sequence identity.” Sequence identity is frequently measured in terms of percentage identity (or similarity or homology); the higher the percentage, the more similar the two sequences are. Homologs or orthologs of human NOTCH receptors, and the corresponding cDNA or gene sequence(s), will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g., human and C. elegans sequences).

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene, 73: 237-244, 1988; Higgins & Sharp CABIOS 5: 151-153, 1989; Comet et al. Nuc. Acids Res. 16, 10881-90, 1988; Huang et al. Computer Appls. in the Biosciences 8:155-65, 1992; and Pearson et al. Meth. Mol. Bio. 24:307-31, 1994. Altschul et al. J. Mol. Biol. 215:403-410, 1990, presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. By way of example, for comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment is performed using the Blast 2 sequences function, employing the PAM30 matrix set to default parameters (open gap 9, extension gap 1 penalties).

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence remains hybridized to a perfectly matched probe or complementary strand. Conditions for nucleic acid hybridization and calculation of stringencies can be found in Sambrook et al. (In Molecular Cloning: A Laboratory Manual, CSHL, New York, 1989) and Tijssen (Laboratory Techniques in Biochemistry and Molecular Biology—Hybridization with Nucleic Acid Probes Part I, Chapter 2, Elsevier, New York, 1993). Nucleic acid molecules that hybridize under stringent conditions to a human NOTCH receptor-encoding sequence will typically hybridize to a probe based on either an entire human NOTCH polynucleotide or selected portions of the NOTCH polynucleotide under wash conditions of 2×SSC at 50° C.

Nucleic acid sequences that do not show a high degree of sequence identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

In the context of the present invention, reference to “at least one,” “at least two,” “at least three,” etc. of the mutations listed in any particular NOTCH receptor means any one or any and all combinations of the mutations listed.

“NOTCH” is a membrane-bound transcription factor that regulates many cellular processes, especially in development. In response to ligand binding, its intracellular domain (ICD) is released by two proteases. The released intracellular domain enters the nucleus and interacts with a DNA-bound protein to activate transcription. The extracellular domain of NOTCH and related proteins contains up to 36 EGF-like domains, followed by three notch (DSL) domains. The intracellular domain (ICD) contains six ankyrin repeats and a carboxyl-terminal extension that includes a PEST domain. The NOTCH1 and NOTCH2 ICD additionally comprises a transactivation domain (TAD). “NOTCH” encompasses all members of the NOTCH receptor family. A description of the NOTCH signaling pathway and conditions affected by it can be found, for example, in WO 98/20142 and WO 00/36089.

A “NOTCH inhibitor,” “NOTCH antagonist,” “anti-NOTCH therapeutic agent,” or “anti-NOTCH agent” as used herein includes any compound that partially or fully blocks, inhibits, or neutralizes a biological activity of the NOTCH pathway. Exemplary NOTCH inhibiting compounds include, but are not limited to gamma-secretase inhibitors such as, III-31-C, N—[N-(3,5-difluorophenacetyl)-L-alanyl]S-phenylglycine t-butyl ester) (DAPT), compound E, D-helical peptide 294, isocoumarins, BOC-Lys(Cbz)Ile-Leu-epoxide, and (Z-LL)₂-ketone (see Kornilova et al., J. Biol. Chem. 278:16479-16473 (2003)); and those compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671 and U.S. Patent Application No. 2003/0114496. Specific gamma secretase inhibitor compounds are also described in U.S. Pat. Nos. 6,984,663 and 7,304,094. Specific antibody NOTCH inhibitors are described herein, as well as in WO 2010/005566, and WO 2010/005567, all of which are herein incorporated by reference. NOTCH inhibitors also include NOTCH ligand antagonists.

“NOTCH inhibitors,” “NOTCH antagonists,” “anti-NOTCH therapeutic agents,” or “anti-NOTCH agents” also encompass antibodies that bind the NOTCH receptor. The term “antibody” means an immunoglobulin molecule that recognizes and specifically binds to a target, such as a protein, polypeptide, peptide, carbohydrate, polynucleotide, lipid, or combinations of the foregoing through at least one antigen recognition site or antigen-binding site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen recognition site of an antibody, and any other modified immunoglobulin molecule comprising an antigen recognition site so long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy chain constant domains referred to as alpha, delta, epsilon, gamma, and mu, respectively. The different classes of immunoglobulins have different and well known subunit structures and three-dimensional configurations. Antibodies can be naked or conjugated to other molecules including, but not limited to, toxins and radioisotopes.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

The term “monoclonal antibody” refers to a homogeneous antibody population involved in the highly specific recognition and binding of a single antigenic determinant, or epitope. This is in contrast to polyclonal antibodies that typically include a mixture of different antibodies directed against different antigenic determinants. The term “monoclonal antibody” encompasses both intact and full-length monoclonal antibodies as well as antibody fragments (such as Fab, Fab′, F(ab′)2, Fv fragments), single chain Fv (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, hybridoma production, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences.

The term “human antibody” means an antibody produced by a human or an antibody having an amino acid sequence corresponding to an antibody produced by a human made using any technique known in the art. This definition of a human antibody includes intact or full-length antibodies, and fragments thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired specificity, affinity, and/or capability while the constant regions are homologous to the sequences in antibodies derived from another species (usually human) to avoid eliciting an immune response in that species.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids (often referred to as “linear epitopes”) and noncontiguous amino acids juxtaposed by tertiary folding of a protein (often referred to as “conformation epitopes”). Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

The terms “specifically binds” or “specific binding” mean that a binding agent or an antibody reacts or associates more frequently, more rapidly, with greater duration, with greater affinity, or with some combination of the above to an epitope or protein than with alternative substances, including unrelated proteins. In certain embodiments, “specifically binds” means, for instance, that an antibody binds to a protein with a KD of about 0.1 mM or less, but more usually less than about 1 μM. In certain embodiments, “specifically binds” means that an antibody binds to a protein at times with a KD of at least about 0.1 μM or less, and at other times at least about 0.01 μM or less.

As used herein, the term “stratifying” refers to sorting subjects into different classes or strata based on the features of a particular disease state or condition. For example, stratifying a population of subjects with NOTCH mediated cancer involves assigning the subjects on the presence of a mutation (tumor classification) and/or on the basis of the severity of the disease (e.g., mild, moderate, advanced, etc.).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. A “normal” subject or sample from a “normal” subject as used herein for quantitative and qualitative data refers to a subject who has or would be assessed by a physician as not having a NOTCH mediated cellular proliferative disorder or a disorder characterized by aberrant NOTCH signaling.

A “control sample” means a separate sample from a comparable control cell, which is generally disease free. It can be from the same subject or from another subject who is normal or does not present with the same disease from which the diseased or test sample is obtained.

The term “prognosis” is used herein to refer to the prediction of the likelihood of cancer attributable death or progression, including recurrence, metastatic spread, and drug resistance, of a neoplastic disease, such as a NOTCH-mediated cancer. As used herein, the term “predicting” or “prediction” refers to making a finding that a subject has a significantly enhanced or reduced probability of an outcome—favorable prognosis versus an unfavorable prognosis. It can also include the likelihood that a NOTCH inhibitor may be therapeutically effective versus one that is not found to be therapeutic. The term may also be used to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. Towards this end, the predictive methods of the present invention are valuable tools in predicting if a patient is likely to respond favorably to a NOTCH-based treatment regimen, such as chemotherapy with a given drug or drug combination, e.g. gamma secretase inhibitor or another NOTCH inhibitor, or whether long-term survival of the patient, following a treatment protocol with a NOTCH inhibitor and/or termination of chemotherapy or other treatment modalities is likely.

The terms “cancer” and “cancerous” refer to or describe the physiological condition in mammals that is typically characterized by unregulated cell growth.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. “Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

“Neoadjuvant therapy” is adjunctive or adjuvant therapy given prior to the primary (main) therapy. Neoadjuvant therapy includes, for example, chemotherapy, radiation therapy, and hormone therapy. Thus, chemotherapy can be administered prior to surgery to shrink the tumor, so that surgery can be more effective, or in the case of previously unoperable tumors, possible.

The term “responsive” as used herein means that a patient or tumor shows a complete response or a partial response after administering an agent, according to RECIST (Response Evaluation Criteria in Solid Tumors). The term “nonresponsive” as used herein means that a patient or tumor shows stable disease or progressive disease after administering an agent, according to RECIST. RECIST is described, e.g., in Therasse et al., February 2000, “New Guidelines to Evaluate the Response to Treatment in Solid Tumors,” J. Natl. Cancer Inst. 92(3): 205-216, which is incorporated by reference herein in its entirety. Exemplary agents include specific binding agents to a NOTCH polypeptide, including but not limited to, antibodies to NOTCH.

A “disorder” is any condition that would benefit from one or more treatments. This includes chronic and acute disorders or disease including those pathological conditions which predispose the mammal to the disorder in question. Non-limiting examples of disorders to be treated herein include benign and malignant tumors, in particular breast, rectal, ovarian, stomach, endometrial, salivary gland, kidney, colon, thyroid, pancreatic, prostate or bladder cancer.

“Treatment” or “therapy” refer to both therapeutic treatment and prophylactic or preventative measures. The term “therapeutically effective amount” refers to an amount of a drug effective to treat a disease or disorder in a mammal. In the case of cancer, the therapeutically effective amount of the anti-NOTCH treatment may, by way of non-limiting example, reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing tumor burden or volume, the time to disease progression (TTP) and/or determining the response rates (RR).

### II. Identification of Activating NOTCH Mutations

Disclosed herein are mutations in NOTCH receptors that cause production of activated NOTCH proteins, which in turn leads to increased receptor signaling. These mutations are linked to neoplastic disease such as cancer, and thereby can be used to assess, for example, whether a cancer patient will be responsive to NOTCH antagonist therapy.

There are four members of the NOTCH family in mammals: NOTCH1 (TAN1), NOTCH2, NOTCH3 and NOTCH4/Int-4. Exemplary sequences for the human NOTCH proteins include, but are not limited to: human NOTCH1, is encoded by the mRNA sequence set forth as Genbank Acc. No. NM_{—}017617.3, and has the amino acid sequence set forth as Genbank Acc. No. NP_{—}060087; human NOTCH2, is encoded by the mRNA sequence set forth as Genbank Acc. No. NM_{—}024408, and has the amino acid sequence set forth as Genbank Acc. No. NP_{—}077719; human NOTCH3, is encoded by the mRNA sequence of Genbank Acc. No. NM_{—}000435.2, and has the amino acid sequence of Genbank Acc. No. NP_{—}000426; and human NOTCH4, is encoded by the mRNA sequence of Genbank Acc. No. NM_{—}004557, and has the amino acid sequence of Genbank Acc. No. NP_{—}004548. Representative wild-type sequences of NOTCH1-4 are shown in SEQ ID NOs:1-4, respectively. The position of the NOTCH1 mutations provided herein were determined using NM_{—}017617.3 as a reference sequence. The position of the NOTCH3 mutations provided herein were determined using NM_{—}000435.2 as a reference sequence. Nucleotide positions are numbered starting at the NOTCH starting codon wherein the adenine of the starting ATG codon (e.g., ---residue 1 of NM_{—}017617.3 and residue 77 of NM_{—}000435.2) corresponds to position 1.

NOTCH is expressed on cell surfaces as a single-pass, heterodimeric receptor. The ligands are also transmembrane proteins of the DSL (Delta/Serrate/LAG-2) family that can be expressed not only on adjacent cells but also on the very same cell expressing the NOTCH receptors. Receptor-ligand interaction triggers proteolysis at the extracellular S2 site near the transmembrane domain and at the S3 site. A TNF-α converting enzyme (TACE) and a presenillin-1-dependent gamma-secretase are believed to be responsible for the proteolytic processing at sites S2 and S3, respectively. The final cleavage releases the carboxy-terminal, intracellular domain (ICD) comprising seven ankyrin repeats flanked by nuclear localization signals, aproline, glutamine, serine, threonine-rich (PEST) domain and a transactivation domain (TAD). The ICD then translocates to the nucleus, recruits coactivators such as mastermind and p300, and binds to CSL (CBF/Suppressor of Hairless/LAG-1) factors. In the absence of NOTCH signaling, CSL proteins in association with corepressors repress target gene transcription. Thus, NOTCH signaling causes a switch from transcriptional repression to transcriptional activation of CSL target genes.

Thus, in one embodiment, the invention is directed to the identification of tumor cells, at least a portion of which contain an activating NOTCH mutation that results in increased NOTCH signaling. In one embodiment, the mutation is of the PEST domain of NOTCH. PEST domain mutations are those which occur within the minimal PEST domain itself, as well as those that occur upstream of the domain and result in either the elimination of the PEST domain (e.g. insertion of a stop codon), or a frameshift that would change the sequence of the PEST domain. The minimal PEST domain sequences are shown in Table 1. In another embodiment, the mutation is of the transactivation domain (TAD) of NOTCH.

TABLE 1
Minimal NOTCH Receptor PEST domains

| | Reference | | Nucleotide | Amino acid |
|---|---|---|---|---|
| Source | Sequence | AA sequence | position | position |
| NOTCH1 | CCDS43905 | FLTPSPESP | 7525-7550 | 2509-2517 |
| | | (SEQ ID NO: 33) | | |
| NOTCH2 | CCDS908 | YLTPSPESP | 7240-7266 | 2414-2422 |
| | | (SEQ ID NO: 34) | | |
| NOTCH3 | CCDS12326 | YLTPSPESP | 6730-6756 | 2244-2252 |
| | | (SEQ ID NO: 34) | | |
| NOTCH4 | NM_004557.3 | LTPSPE | 5914-5931 | 1972-1977 |
| | | (SEQ ID NO: 35) | | |

In another embodiment, the invention is directed to an isolated mutant human NOTCH1 polynucleotide comprising a heterozygous deletion at nucleotide site 7279. In a further embodiment, the mutant NOTCH1 comprises a heterozygous deletion of guanine (G) at the nucleotide site 7279 (RefSeq NM_{—}017617.3; SEQ ID NO:1). This NOTCH1 mutation is herein referred to as OMP-B40. The OMP-B40 mutation causes a reading frame shift in PEST (G2427fs) domain of NOTCH1.

In another embodiment, the invention is directed to an isolated mutant human NOTCH3 polynucleotide comprising a homozygous insertion at nucleotide site 6622. In a further embodiment, the mutant NOTCH3 comprises a homozygous insertion of a cytosine (C) at nucleotide site 6622 (RefSeq NM_{—}000435.2; SEQ ID NO:3). This NOTCH3 mutation is herein referred to as OMP-B37. The insertion causes a reading frame shift at amino acid position 2208 (P2208fs) of the PEST domain.

In another embodiment, the invention is directed to an isolated mutant human NOTCH3 polynucleotide comprising a heterozygous insertion at nucleotide site 6096. In a further embodiment, the mutant NOTCH3 comprises a heterozygous insertion of a cytosine (C) at nucleotide site 6096 (RefSeq NM_{—}000435.2; SEQ ID NO:3). This NOTCH3 mutation is herein referred to as OMP-C31. The insertion causes a reading frame shift at amino acid position 2033 (P2033fs) in the ANK domain.

In another embodiment, the invention is directed to an isolated mutant human NOTCH1 polynucleotide comprising a heterozygous substitution at nucleotide site 6733. In a further embodiment, the mutant NOTCH1 comprises a heterozygous substitution of a guanine (G) to adenine (A) at nucleotide site 6733 (RefSeq NM_{—}017617.3; SEQ ID NO:1). This NOTCH1 mutation is herein referred to as Lung_{—}01246. The substitution causes a missense mutation of Gly to Arg at amino acid position 2245 (G2245R) of the TAD domain.

In another embodiment, the invention is directed to an isolated mutant human NOTCH1 polynucleotide comprising a heterozygous substitution at nucleotide site 6788. In a further embodiment, the mutant NOTCH1 comprises a heterozygous substitution of a cytosine (C) to thymine (T) at nucleotide site 6788 (RefSeq NM_{—}017617.3; SEQ ID NO:3). This NOTCH1 mutation is herein referred to as Breast_H12932T. The substitution causes a missense mutation of Arg to Gln at amino acid position 2263 (R2263Q) of the TAD domain.

TABLE 2
NOTCH Mutations

| | NOTCH | Amino | Nucleotide Sequence |
|---|---|---|---|
| Mutation | Receptor | Acid Position | Variation |
| B40 | NOTCH1 | Frameshift at | heterozygous deletion |
| | | 2427 | of guanine (G) at the |
| | | | nucleotide 7279 |
| B37 | NOTCH3 | Frameshift at | insertion of cytosine (C) |
| | | 2208 | at nucleotide site 6622 |
| C31 | NOTCH3 | Frameshift at | heterozygous insertion |
| | | 2033 | of cytosine (C) at |
| | | | nucleotide site 6096 |
| Lung_01246 | NOTCH1 | Missense | heterozygous nucleotide |
| | | G2245R | substitution guanine (G) |
| | | | to adenine (A) at |
| | | | nucleotide site 6733 |
| Breast_H12932T | NOTCH1 | Missense | heterozygous nucleotide |
| | | R2263Q | substitution cytosine (C) |
| | | | to thymidine (T) at |
| | | | nucleotide 6788 |

The polypeptides of the invention can be cloned using DNA amplification methods, such as the polymerase chain method (PCR) (see e.g., Sambrook et al. (1989) Molecular Cloning: A Laboratory Manual, 2nd ed., Cold Spring Harbour, N.Y.; Berger & Kimmel (1987) Methods in Enzymology. Vol. 152). Thus, for example, a nucleic acid molecule encoding a mutant NOTCH polypeptide can be PCR amplified using a sense primer containing one restriction site and an antisense primer containing another restriction site. This will produce a nucleic acid encoding the desired sequence or subsequence having terminal restriction sites. This nucleic acid can then easily be ligated into a vector having appropriate corresponding restriction sites. Suitable PCR primers are easily chosen by one of skill in the art based on the sequence to be expressed. Appropriate restriction sites can also be added by site-directed mutagenesis (see Gillman & Smith Gene 8: 81-97(1979); Roberts et al. Nature 328: 731-4 (1987)).

The methods of introducing exogenous nucleic acid into host cells are well known in the art, and will vary with the host cell used. Suitable techniques include, but are not limited to, dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In certain embodiments, the polynucleotides comprise the coding sequence for the chimeric polypeptide fused in the same reading frame to a polynucleotide which aids, for example, in expression and secretion of a polypeptide from a host cell (e.g. a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g. COS-7 cells) is used.

The mutant polypeptides of the invention are typically expressed using an expression vector and purified. Expression vectors can be either self-replicating extrachromosomal vectors or vectors which integrate into a host genome. Generally, expression vectors include transcriptional and translational regulatory nucleic acid sequences operably linked to the nucleic acid encoding the target protein. Operably linked DNA sequences can be contiguous or non-contiguous. Methods for linking DNA sequences are well-known in the art and include use of the polymerase chain reaction and ligation. The transcriptional and translational regulatory nucleic acid will generally be appropriate to the host cell used to express the target protein; for example, transcriptional and translational regulatory nucleic acid sequences from E. coli are preferably used to express the target protein in E. coli.

Numerous types of appropriate expression vectors, and suitable regulatory sequences are known in the art for a variety of host cells. Methods for expressing polypeptides are well known in the art (e.g., Sambrook et al. (1989) Molecular Cloning, A Laboratory Manual, 2nd ed., vol. 1-3, Cold Spring Harbor Laboratory; Berger and Kimmel (1987) Guide to Molecular Cloning Techniques, Methods in Enzymology, vol. 152, Academic Press, Inc., San Diego, Calif.; Ausubel et al. (1995) Current Protocols in Molecular Biology, John Wiley & Sons, Inc., NY).

In general, the transcriptional and translational regulatory sequences can include, but are not limited to, promoter sequences, ribosomal binding sites, transcriptional start and stop sequences, translational start and stop sequences, and enhancer or activator sequences. Promoter sequences encode either constitutive or inducible promoters. The promoters can be either naturally occurring promoters or hybrid promoters. Hybrid promoters, which combine elements of more than one promoter, are also known in the art.

An expression vector can comprise additional elements. For example, the expression vector can have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to a sequence in the host cell genome, and preferably two homologous sequences that flank the expression construct. The integrating vector can be directed to a specific locus in the host cell by selecting the appropriate homologous sequence for inclusion in the vector. Constructs for integrating vectors are well known in the art.

In addition, an expression vector can include a selectable marker gene to allow the selection of transformed host cells. Selection genes are well known in the art and will vary depending on the host cell used.

The polypeptides of the invention can be produced by culturing a host cell transformed with an expression vector containing nucleic acid encoding a mutant NOTCH polypeptide, under the appropriate conditions to induce or cause expression of the mutant polypeptide. The conditions appropriate for protein expression will vary with the choice of the expression vector and the host cell, and will be easily ascertained by one skilled in the art using routine experimentation. For example, the growth and proliferation of the host cell can be optimized for the use of constitutive promoters in the expression vector, and appropriate growth conditions for induction are provided for use of an inducible promoter. In addition, in some embodiments, the timing of the harvest is important, for example, when using baculoviral systems. One of skill in the art will recognize that the coding sequences can be optimized for expression in the selected host cells.

Appropriate host cells include yeast, bacteria, archaebacteria, fungi, and insect and animal cells, including mammalian cells. Host cells include, but are not limited to, Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, Hep G2 cells, and human cells and cell lines.

The mutant NOTCH polypeptides of the invention can also be made as fusion proteins, using techniques that are well known in the art. For example, a NOTCH polypeptide can be made as a fusion protein to increase expression, to increase serum half-life, or to link it with a tag polypeptide that provides an epitope to which an anti-tag antibody can selectively bind. Exemplary tags or fusion partners include the myc epitope, the immunoglobulin Fc domain, and 6-histidine. The epitope tag is generally placed at the amino- or carboxyl-terminus of the target protein. The presence of such epitope-tagged forms of a target protein can be detected using an antibody against the tag polypeptide. Thus, the epitope tag enables the target proteins to be readily purified by affinity purification using an anti-tag antibody or another type of affinity matrix that binds to the epitope tag.

The NOTCH polypeptides of the invention can be purified or isolated after expression. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. For example, it means that the protein is at least 85% pure, such as at least 95% pure, such as at least 99% pure.

The NOTCH polypeptides of the invention can be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the target protein can be purified using an affinity column. Ultrafiltration and diafiltration techniques, in conjunction with protein concentration, are also useful. Suitable purification techniques are standard in the art (see generally R. Scopes (1982) Protein Purification, Springer-Verlag, N.Y.; Deutcher (1990) Methods in Enzymology vol. 182: Guide to Protein Purification, Academic Press, Inc. N.Y.). The degree of purification necessary will vary depending on the use of the polypeptide. In some instances no purification is necessary.

In certain embodiments, a NOTCH polynucleotide or polypeptide can comprise a heterologous amino acid sequence or one or more other moieties not normally associated with a NOTCH polypeptide (e.g., an antimicrobial agent, a therapeutic agent, a prodrug, a peptide, a protein, an enzyme, a lipid, a biological response modifier, pharmaceutical agent, a lymphokine, a heterologous antibody or fragment thereof, a detectable label, polyethylene glycol (PEG), and a combination of two or more of any the agents). In further embodiments, a NOTCH polynucleotide or polypeptide can comprise a detectable label selected from the group consisting of an enzyme, a fluorescent label, a chemiluminescent label, a bioluminescent label, a radioactive label, or a combination of two or more of any the detectable labels.

The present invention also encompasses antibodies that bind specifically to mutant forms of NOTCH receptors and processes for producing such antibodies. Antibodies that “bind specifically to mutant NOTCH receptors” are defined as those that have at least a hundred-fold greater affinity for the mutant form of the receptor than for the wild-type form. The process for producing such antibodies may involve either injecting the mutant receptor protein into an appropriate animal or, preferably, injecting short peptides that include regions where mutations occur. The peptides should be at least five amino acids in length and may be injected either individually or in combinations.

Methods for making and selecting antibodies are well known to those of skill in the art as evidenced by standard reference works such as Harlow, et al., Antibodies, A Laboratory Manual, Cold Spring Harbor Laboratory, N.Y. (1988); Klein, Immunology: The Science of Self-Nonself Discrimination (1982); Kennett, et al., Monoclonal Antibodies and Hybridomas: A New Dimension in Biological Analyses (1980); and Campbell, “Monoclonal Antibody Technology,” in: Laboratory Techniques in Biochemistry and Molecular Biology (1984).

The term “antibody,” as used herein, is meant to include intact molecules as well as fragments that retain their ability to bind antigen, such as Fab and F(ab)₂ fragments. Polyclonal antibodies are derived from the sera of animals immunized with an appropriate antigen. Monoclonal antibodies can be prepared using hybridoma technology as taught by references such as: Hammerling, et al., in Monoclonal Antibodies and T-Cell Hybridomas, Elsevier, N.Y., pp. 563-681 (1981). In general, this technology involves immunizing an immunocompetent animal, typically a mouse, with either intact protein or a fragment derived therefrom. Splenocytes are then extracted from the immunized animal and are fused with suitable myeloma cells, such as SP2O cells. Thereafter, the resulting hybridoma cells are selectively maintained in HAT medium and then cloned by limiting dilution (Wands et al., Gastroenterology 80:225-232 (1981)). The cells obtained through such selection may then be assayed to identify clones that secrete antibodies binding preferentially to mutant forms of NOTCH1, NOTCH3, or mutant forms of other NOTCH receptors.

Antibodies to mutant NOTCH receptors may also be used in the purification of either the intact receptor or fragments of these receptors (see generally, Dean, et al., Affinity Chromatograph, A Practical Approach, IRLP Press (1986)). Typically, antibody is immobilized on a chromatographic matrix such as Sepharose 4B. The matrix is then packed into a column and a preparation containing mutant receptor is passed through the column under conditions that promote binding, e.g., under conditions of low salt. The column is then washed and bound receptor is eluted using a buffer that promotes dissociation from antibody (e.g., a buffer having an altered pH or salt concentration). The eluted receptor protein can be transferred into a buffer of choice, e.g., by dialysis and either stored or used directly. Purified receptor can be used in the immunoassays described below or for the generation of antibodies for use in assays.

The discovery of activating mutations in the sequence of NOTCH receptors enables a variety of diagnostic, prognostic, and therapeutic methods that are further embodiments. The identification of activating mutations also enables early identification of subjects with tumors particularly sensitive to NOTCH antagonist therapy. Identification of the activating mutations described herein provides opportunities for early treatment as well as particular treatment selection.

### III. Diagnostic Applications

When present in a cancer, mutant isoforms of NOTCH represent a therapeutic target for NOTCH inhibitors, immunotherapy, and other novel targeted approaches. Because activated NOTCH mutations are not found in all tumors, the selection of patients for therapy targeting mutant NOTCH receptors would be optimized by pre-therapy analysis of cancer cells for the presence of NOTCH gene mutations.

Methods for detecting a NOTCH mutation of the invention comprise any method that determines the presence of the mutation at either the nucleic acid or protein level. Such methods are well known in the art and include but are not limited to western blots, northern blots, southern blots, ELISA, immunoprecipitation, immunofluorescence, flow cytometry, immunocytochemistry, nucleic acid sequencing, nucleic acid hybridization techniques, nucleic acid reverse transcription methods, and nucleic acid amplification methods, such as PCR. In certain embodiments, the diagnostic analyses can be based on PCR-based assays for these mutations, using for instance one or more of the following approaches: size fractionation by gel electrophoresis, direct sequencing, single-strand conformation polymorphism (SSCP), high pressure liquid chromatography (including partially denaturing HPLC), allele-specific hybridization, amplification refractory mutation screening, NOTCH mutation screening by oligonucleotide microarray, restriction fragment polymorphism, MALDI-TOF mass spectrometry, or various related technologies (Abu-Duhier et al., Br. J. Haematol., 113: 983-988, 2001; Kottaridis et al., Blood, 98: 1752-1759, 2001; Choy et al., Ann. Hum. Gen., 63: 383-391, 1999; Grompe, Nature Genetics, 5: 111-117, 1993; Perlin & Szabady, Hum. Mutat., 19: 361-373, 2002; Amos & Patnaik, Hum. Mutat., 19: 324-333, 2002; Cotton, Hum. Mutat., 19: 313-314, 2002; Stirewalt et al., Blood, 97: 3589-3595, 2001; Hung et al., Blood Coagul. Fibrinolysis, 13: 117-122, 2002; Larsen et al., Pharmacogenomics, 2: 387-399, 2001; Shchepinov et al., Nucleic Acids Res., 29: 3864-3872, 2001).

In particular embodiments, mutations in the NOTCH proteins (which result in increased NOTCH signaling) is detected on a protein level using, for example, antibodies that are directed against mutated NOTCH receptors, or downstream NOTCH signaling proteins. These antibodies can be used in various methods such as Western blot, ELISA, immunoprecipitation, or immunocytochemistry techniques.

Immunoassays

In one embodiment, antibodies specific for mutant NOTCH proteins are used to detect the presence of the NOTCH mutations in a body sample. The phrase “body sample” as used herein, is intended any sample comprising a cell, a tissue, or a bodily fluid in which the presence of the NOTCH mutation can be detected. Examples of such body samples include, but are not limited to, blood, lymph, urine, gynecological fluids, biopsies, amniotic fluid and smears. Body samples can be obtained from a patient by a variety of techniques. Methods for collecting various body samples are well known in the art. In some embodiments, the method comprises obtaining a body sample from a patient, and contacting the body sample with at least one antibody directed to a mutant NOTCH protein. Such immunoassay methods can be performed manually or in an automated fashion.

Techniques for detecting antibody binding are well known in the art. Antibody binding to a mutant NOTCH protein can be detected through the use of chemical reagents that generate a detectable signal that corresponds to the level of antibody binding and, accordingly, to the presence of a mutated NOTCH protein. In one embodiment, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the mutation of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), can be used to practice the present invention.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Immunoassays, in their simplest and most direct sense, are binding assays. In some embodiments, immunoassays are the various types of enzyme linked immunosorbent assays (ELISA) and radioimmunoassays (RIA) known in the art. It will be readily appreciated that detection is not limited to such techniques, and western blotting, dot blotting, FACS analyses, and the like may also be used.

Nucleic Acid-Based Techniques

In other embodiments, the presence of the NOTCH mutations is detected at the nucleic acid level. Nucleic acid-based techniques for assessing expression and identification of NOTCH mutations are well known in the art. In one embodiment, the NOTCH mutations are identified by direct nucleic acid sequencing. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski (U.S. Pat. No. 4,843,155).

The term “probe” refers to any molecule that is capable of selectively binding to a specifically intended target biomolecule, for example, a nucleotide transcript or a protein encoded by or corresponding to a mutant NOTCH protein. Probes can be synthesized by one of skill in the art using known techniques, or derived from appropriate biological preparations. Probes may be specifically designed to be labeled with a detectable label. Examples of molecules that can be used as probes include, but are not limited to, RNA, DNA, proteins (including peptides), antibodies, and organic molecules.

Isolated mRNA from mutant NOTCH proteins can be detected in hybridization or amplification assays that include, but are not limited to, mRNA sequencing methods, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a mutant NOTCH polypeptide of the present invention. Hybridization of an mRNA with the probe indicates that the mutation in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). Known mRNA detection methods can be readily adapted for use in detecting the presence of NOTCH mutations of the present invention.

An alternative method for determining the presence of mutant NOTCH mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the presence of NOTCH mutations is assessed by quantitative fluorogenic RT-PCR (i.e., the TaqMan® System). Such methods typically use pairs of oligonucleotide primers that flank regions comprising the NOTCH mutations of interest. Methods for designing oligonucleotide primers specific for a known sequence are known in the art.

Microarray

In one embodiment of the invention, microarrays are used to detect the presence of NOTCH mutations in biological samples. Microarrays are particularly well suited for this purpose because of their reproducibility. DNA microarrays provide one method for the simultaneous measurement of the expression levels of large numbers of genes or a large number of oligonucletide probes directed to different parts of a molecule of interest. Each array consists of a reproducible pattern of capture probes attached to a solid support. Labeled RNA or DNA is hybridized to complementary probes on the array and then detected by for example, laser scanning.

Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNAs in a sample.

Techniques for the synthesis of these arrays using mechanical synthesis methods are described in, e.g., U.S. Pat. No. 5,384,261, incorporated herein by reference in its entirety. Although a planar array surface is preferred, the array can be fabricated on a surface of virtually any shape or even a multiplicity of surfaces. Arrays can be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety. Arrays can be packaged in such a manner as to allow for diagnostics or other manipulation of an all-inclusive device. See, for example, U.S. Pat. Nos. 5,856,174 and 5,922,591 herein incorporated by reference.

Nucleic acids which code for the mutant NOTCH proteins can be placed in an array on a substrate, such as on a chip (e.g., DNA chip or microchips). These arrays also can be placed on other substrates, such as microtiter plates, beads or microspheres. Methods of linking nucleic acids to suitable substrates and the substrates themselves are described, for example, in U.S. Pat. Nos. 5,981,956; 5,922,591; 5,994,068 (Gene Logic's Flow-thru ChipO Probe ArraysO); U.S. Pat. Nos. 5,858,659, 5,753,439; 5,837,860 and the FlowMetrix technology (e.g., microspheres) of Luminex (U.S. Pat. Nos. 5,981,180 and 5,736,330).

Screening for multiple mutations in samples of genomic material according to the methods of the present invention, is generally carried out using arrays of oligonucleotide probes. These arrays may generally be “tiled” for a large number of specific mutations. By “tiling” is generally meant the synthesis of a defined set of oligonucleotide probes which is made up of a sequence complementary to the target sequence of interest, as well as pre-selected variations of that sequence, e.g., substitution of one or more given positions with one or more members of the basic set of monomers. i.e. nucleotides. Tiling strategies are discussed in detail in Published PCT Application No. WO 95/11995, incorporated herein by reference in its entirety for all purposes. By “target sequence” is meant a sequence which has been identified as encoding a mutant polypeptide of interest or portion thereof

In a particular aspect, arrays are tiled for a number of specific, identified NOTCH mutations. In particular, the array is tiled to include a number of detection blocks, each detection block being specific for a particular mutation or set of mutations. For example, a detection block can be tiled to include a number of probes which span the sequence segment that includes a specific mutation or set of mutations. To ensure probes that are complementary to each variant, the probes are synthesized in pairs differing, for example, at the biallelic base.

Optimal tiling configurations can be determined for any particular mutation by comparative analysis. For example, triplet or larger detection blocks can be readily employed to select such optimal tiling strategies.

Additionally, arrays will generally be tiled to provide for ease of reading and analysis. For example, the probes tiled within a detection block will generally be arranged so that reading across a detection block the probes are tiled in succession, i.e., progressing along the target sequence one or more nucleotides at a time.

Once an array is appropriately tiled for a set of mutations, the target nucleic acid is hybridized with the array and scanned. A target nucleic acid sequence, which includes one or more previously identified mutations, is amplified by well-known amplification techniques, e.g., polymerase chain reaction (PCR). Typically, this involves the use of primer sequences that are complementary to the two strands of the target sequence both upstream and downstream from the mutation. Asymmetric PCR techniques can also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single mutation, in some embodiments, the arrays of the invention will include multiple detection blocks, and thus be capable of analyzing multiple, specific mutations.

In alternate arrangements, it will generally be understood that detection blocks can be grouped within a single array or in multiple, separate arrays so that varying, optimal conditions may be used during the hybridization of the target to the array. For example, it may often be desirable to provide for the detection of those polymorphisms that fall within G C rich stretches of a genomic sequence, separately from those falling in A T rich segments. This allows for the separate optimization of hybridization conditions for each situation.

In one approach, total mRNA isolated from the sample is converted to labeled cRNA or cDNA and then hybridized to an oligonucleotide array containing one or more mutations of the invention. Each sample is hybridized to a separate array. Relative transcript levels can be calculated by reference to appropriate controls present on the array and in the sample.

In addition to direct detection of mutant NOTCH proteins, it is expected that the activated NOTCH mutants will result in distinctive signal transduction profiles (e.g. increased signaling) that could be detected by global gene expression profile or analysis of the activation of various signaling intermediates.

While individual mutations are useful diagnostic biomarkers, in one embodiment a combination of mutations can be used to provide predictive value of a particular therapeutic status. Specifically, the detection of a plurality of mutations in a sample can increase the sensitivity and/or specificity of the test. A combination of at least two mutations is sometimes referred to as a “mutation profile” or “mutation fingerprint.”

The pathogenesis of a subset of malignancies, including both hematological and solid tumors, has been linked to increased NOTCH mediated signaling in the tumor cells. Increased NOTCH mediated signaling can be associated with the presence of activating NOTCH mutations, for example, the NOTCH mutations described herein. It can also be associated with mutations that reduce or eliminate the activity of negatively regulators of NOTCH signaling. See, e.g., Westhoff et al., Proc Natl Acad Sci 2009 Dec. 29; 106(52): 22293-22298. Increased NOTCH mediated signaling can be associated with overexpression of the NOTCH ICD. Because activated NOTCH signaling is not associated with all tumors, the selection of patients for therapy targeting NOTCH signaling would be optimized by pre-therapy analysis of cancer cells for the presence of increased levels of NOTCH ICD.

Methods for detecting the level of NOTCH ICD in tumor cells can comprise any method that detects the presence of a NOTCH ICD polypeptide in a biological sample. Such methods are well known in the art and include, but are not limited to, western blots, slot blots, ELISA, immunoprecipitation, immunofluorescence, flow cytometry, immunocytochemistry, immunohistochemistry (IHC), and mass spectroscopy. In one embodiment, the level of NOTCH ICD in a tumor sample is determined using IHC.

In one embodiment, the level of NOTCH ICD is determined using an agent the specifically binds to NOTCH ICD. Any molecular entity that displays specific binding to NOTCH ICD can be employed to determine the level of NOTCH ICD in a sample. Specific binding agents include, but are not limited to, antibodies, antibody mimetics, and polynucleotides (e.g., aptamers). One of skill understands that the degree of specificity required is determined by the particular assay used to detect NOTCH ICD. For example, an agent that specifically binds to both full length NOTCH and NOTCH ICD can be used in a method that involves the separation of polypeptides based on their size, e.g. Western blot.

In one embodiment, a method employs an agent that specifically binds to NOTCH1 ICD, NOTCH2 ICD, NOTCH3 ICD or NOTCH4 ICD to determine the level of NOTCH1 ICD, NOTCH2 ICD, NOTCH3 ICD or NOTCH4 ICD, respectively. In another embodiment, a method employs an agent that specifically binds to at least two of, at least three of, or all four of NOTCH1 ICD, NOTCH2 ICD, NOTCH3 ICD and NOTCH4 ICD to determine the combined level of NOTCH ICDs specifically bound by the agent. In one embodiment, the method employs an agent that specifically binds to NOTCH1 ICD to determine the level of NOTCH1 ICD in a sample. In a further embodiment, the method employs an agent that specifically binds to NOTCH3 ICD to determine the level of NOTCH3 ICD in a sample.

In one embodiment, the level of NOTCH ICD is determined using an antibody specific for NOTCH ICD. In another embodiment, the antibody is a monoclonal antibody. Anti-NOTCH ICD-specific antibodies can be generated according to any method known to one of skill in the art. See, e.g., Tagami et al., Mol. Cell. Biol. 28(1):165-176. Anti-NOTCH ICD specific antibodies are also available from commercial sources. See, e.g., R&D Systems, Anti-human NOTCH-2 Intracellular Domain Antibody, Catalog # BAF3735. In one embodiment, an anti-NOTCH ICD specific antibody specifically binds to NOTCH ICD but does not significantly bind to NOTCH. In another embodiment, an anti-NOTCH ICD specific antibody specifically binds to NOTCH1 ICD, NOTCH2 ICD, NOTCH3 ICD or NOTCH4 ICD. In another embodiment, an anti-NOTCH ICD specific antibody specifically binds to at least two, at least three or all four of NOTCH1 ICD, NOTCH2 ICD, NOTCH3 ICD or NOTCH4 ICD. The anti-NOTCH ICD antibody can be monoclonal antibody, polyclonal antibody, humanized antibody, human antibody, chimeric antibody or an antigen binding fragment thereof. In a further embodiment, the antibody specifically binds to NOTCH ICD in a fixed and embedded tissue sample. The tissue sample can be a formalin fixed tissue sample. The tissue sample can be a paraffin embedded tissue sample.

In one embodiment, an agent, e.g., antibody, the specifically binds to NOTCH ICD is used to determine the level of NOTCH ICD in a body sample. The phrase “body sample” as used herein, is intended any sample comprising a cell, a tissue, or a bodily fluid in which the level of NOTCH ICD can be determined. Examples of such body samples include, but are not limited to, blood, lymph, urine, gynecological fluids, biopsies, tissues, amniotic fluid, solid tissue samples obtained by surgical removal, pathology specimen, archived sample, tissue cultures or cells derived therefrom and the progeny thereof, and sections or smears prepared from any of these sources. Body samples can be obtained from a patient by a variety of techniques. Methods for collecting various body samples are well known in the art. The term also includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. In some embodiments, the method comprises obtaining a body sample from a patient, and contacting the body sample with at least one antibody that specifically binds to NOTCH ICD. Such immunoassay methods can be performed manually or in an automated fashion.

Techniques for detecting antibody binding are well known in the art. Antibody binding to a NOTCH ICD can be detected through the use of chemical reagents that generate a detectable signal that corresponds to the level of antibody binding and, accordingly, to the level of NOTCH ICD. In one embodiment, NOTCH ICD antibody binding is detected through the use of a secondary antibody that is conjugated to a labeled polymer. Examples of labeled polymers include, but are not limited to, polymer-enzyme conjugates. The enzymes in these complexes are typically used to catalyze the deposition of a chromogen at the antigen-antibody binding site, thereby resulting in cell staining that corresponds to expression level of the mutation of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), can be used in the methods of the invention.

Detection of antibody binding can be facilitated by coupling the NOTCH ICD antibody to a detectable label. Examples of detectable labels include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 35S, or 3H.

The level of antibody binding to NOTCH ICD can be quantified by various methods known in the art, for example, enzyme linked immunosorbent assays (ELISA), immunofluorescence, immunohistochemistry and radioimmunoassay (RIA). The detection of NOTCH ICD levels is not limited to such techniques, and western blotting, dot blotting, FACS analyses, and the like may also be used.

In one embodiment, the method comprises determining the level of NOTCH ICD in a subcellular compartment, for example, in the cytosol or in the nucleus. In one embodiment, a method described herein comprises determining the level of NOTCH ICD in the nucleus. In one embodiment, the level of nuclear NOTCH ICD can be determined by isolating the nuclear protein fraction from a sample. In another embodiment, the level of nuclear NOTCH ICD is determined by microscopy. Such nuclear NOTCH ICD determination methods can be performed manually or in an automated fashion.

In one embodiment, the level of NOTCH ICD in tumor cell nuclei is determined by microscopy. NOTCH ICD levels can be determined, for example, by immunofluorescence, immunohistochemistry and radioimmunoassay. In one embodiment, the level of nuclear NOTCH ICD is determined by immunohistochemistry (IHC). The level of nuclear NOTCH ICD in a sample can be expressed by any scoring system known to the skilled artisan.

The level of NOTCH ICD in a tumor sample may be scored based on the intensity of the NOTCH ICD specific staining or based on the percentage of NOTCH ICD positive cells. In one embodiment, the level of nuclear NOTCH ICD in a tumor sample is expressed as a proportion, e.g., percentage, of cells within the sample that comprise detectable amounts of NOTCH ICD. For example, the level of nuclear NOTCH ICD in a sample can be expressed as 10%, 20%, 30%, 40%, etc. of the nuclei in the tumor sample are NOTCH ICD positive. In another embodiment, the level of nuclear NOTCH ICD in a tumor sample is determined by assessing the staining intensity of the nuclei in the tumor sample. For example, a tumor sample can be characterized as negative, weakly stained, and intermediate or strongly stained based on the NOTCH ICD specific staining intensity of the nuclei.

In a further embodiment, the level of nuclear NOTCH ICD in a tumor sample is determined by assessing both the intensity and frequency of the NOTCH ICD specific. In one embodiment, the H-score is used to characterize the nuclear NOTCH ICD level in a tumor sample. A semi-quantitative intensity scale ranging from 0 for no staining to 3+ for the most intense staining is used to assign a staining intensity score to nuclei in the sample. The percentage of nuclei falling into each category, i.e., 0, 1+, 2+ and 3+ is counted. An H-Score can be calculated for staining of the nuclei using the following formula: H-Score=(% at 0)*0+(% at 1+)*1+(% at 2+)*2+(% at 3+)*3. The H-score produces a continuous variable that ranges from 0 to 300.

Another aspect of the methods described herein is comparing the level of NOTCH ICD detected in a test sample to a predetermined standard or reference level, for example, the NOTCH ICD level of a control sample. A control sample can be a sample obtained from the patient in a manner similar to the test samples wherein the control sample does not comprise tumor cells. A control sample can also be obtained in a manner similar to the test samples from a subject that does not have a tumor or cancer.

In one embodiment, the method comprises comparing the level of NOTCH ICD to a predetermined standard, or reference level, or control level. The terms “predetermined standard,” “reference level,” and “control level” are, in some instances, used interchangeably herein. In one embodiment, a predetermined standard is a baseline level of NOTCH ICD measured in a comparable control sample, e.g., a sample that does not comprise tumor or cancer cells. In another embodiment, a predetermined standard is a baseline level of NOTCH ICD measured in a sample comprising cancer cells that do not express elevated levels of NOTCH ICD. In a further embodiment, a predetermined standard is a baseline level of NOTCH ICD measured in a sample comprising cancer cells that do not respond to treatment with a NOTCH antagonist or inhibitor, e.g., an anti-NOTCH antibody. In another embodiment, a predetermined standard is a baseline level of NOTCH ICD measured in an isolated cell line. The cell line can be derived from a cancer sample. The cell line can be recombinantly manipulated to express NOTCH or NOTH ICD.

In certain alternative embodiments, the reference level or predetermined standard is not based on NOTCH ICD levels in normal cells, but rather the reference level or predetermined standard is based on NOTCH ICD levels in tumor cells.

In some embodiments, the reference level or predetermined standard to which the level of NOTCH ICD (e.g., NOTCH1 ICD) is compared is an H-score value. In some embodiments, the reference level is an H-score of about 10, about 20, about 30, about 50, or about 100. In some embodiments, the reference level is an H-score of about 30. In some embodiments, the H-score is from an immunohistochemical assay with an anti-NOTCH1 ICD antibody (a “NOTCH1 ICD IHC assay”).

In certain embodiments, if a patient's tumor has an H-score of about 30 or more in a NOTCH1 ICD IHC assay, the patient is selected for treatment and/or treated with an anti-NOTCH1 antibody or other anti-NOTCH therapeutic agent described herein. In some such embodiments, the patient is selected for treatment and/or treated with OMP-52M51, or an antibody comprising the six CDRs and/or the variable regions of OMP-52M51. In some alternative embodiments, if a patient's tumor has an H-score of about 50 or more in a NOTCH1 ICD IHC assay, the patient is selected for treatment and/or treated with an anti-NOTCH1 antibody or other anti-NOTCH therapeutic agent described herein. In some such embodiments, the patient is selected for treatment and/or treated with OMP-52M51, an antibody comprising the six CDRs and/or the variable regions of OMP-52M51. In some alternative embodiments, if a patient's tumor has an H-score of about 100 or more in a NOTCH1 ICD IHC assay, the patient is selected for treatment and/or treated with an anti-NOTCH1 antibody or other anti-NOTCH therapeutic agent described herein (including, but not limited to OMP-52M51 or an antibody comprising the six CDRs and/or the variable regions of OMP-52M51.

### IV. Anti-NOTCH Agents

Anti-NOTCH therapeutic agents are antagonists that block or reduce NOTCH signaling or interactions with NOTCH ligands. NOTCH receptor activation relies on ligand-induced proteolysis. Binding of DSL ligands leads to a cleavage at S2 in the extracellular portion of the NTM. Further cleavage by a gamma-secretase complex at site 3 results in release of the intracellular domain of N^{TM} (ICD) allowing for translocation of this domain to the nucleus. In the nucleus, the ICD forms a multiprotein complex that activates transcription of target genes by binding to transcription factors and scaffolding proteins like Mastermind-like-1-3, that recruit coactivators. Nuclear ICD is short-lived, being targeted for destruction through a mechanism involving carboxy-terminal destruction boxes of the PEST domain common to all NOTCH receptors. Thus, anti-NOTCH therapeutic agents can be any agent that inhibits NOTCH activation including, but not limited to, agents that target the ligand binding region and the LNR HD domain. In certain embodiments, the anti-NOTCH therapeutic agents are antibodies, such as antibodies that bind specifically to NOTCH receptors (i.e., anti-NOTCH antibodies). In certain embodiments, the antibodies specifically bind human NOTCH receptors.

Generally, the anti-NOTCH agents target signaling by at least the NOTCH receptor(s) that bears an activating mutation. For example, an anti-NOTCH1 therapeutic agent is used in treatment of patients bearing a NOTCH1 activating mutation.

In another embodiment, the anti-NOTCH agents target signaling by the NOTCH receptor(s) having an ICD expression level above the level in a predetermined standard or reference level. For example, an anti-NOTCH1 therapeutic agent is used in treatment of patients with tumor cells comprising a level of NOTCH1 ICD above a predetermined standard or reference level.

In certain embodiments, the anti-NOTCH agents are inhibitors for gamma-secretase. Because gamma-secretase inhibitors are also able to prevent NOTCH receptor activation, several forms of gamma-secretase inhibitors have been tested for antitumor effects. First, an original gamma-secretase inhibitor, IL-X (cbz-IL-CHO), was shown to have NOTCH1-dependent antineoplastic activity in Ras-transformed fibroblasts. A tripeptide gamma-secretase inhibitor (z-Leu-leu-Nle-CHO) was reported to suppress tumor growth in cell lines and/or xenografts in mice from melanoma and Kaposi sarcoma (Curry C L et al., Oncogene 24:6333-44(2005)). Treatment with dipeptide gamma-secretase inhibitor N—[N-(3,5-difluorophenacetyl)-L-alanyl]-S-phenylglycine t-butyl ester (DAPT) also resulted in a marked reduction in medulloblastoma growth and induced G0-G1 cell cycle arrest and apoptosis in a T-ALL animal model (O'Neil J. et al., Blood 107:781-5 (2006)). Another gamma-secretase inhibitor, dibenzazepine, has been shown to inhibit epithelial cell proliferation and induce goblet cell differentiation in intestinal adenomas in Apc−/− (min) mice (van Es J H, et al., Nature 435:959-63 (2005)). More recently, functional inactivation of NOTCH3 either by tripeptide gamma-secretase inhibitor or NOTCH3-specific small interfering RNA results in suppression of cell proliferation and induction of apoptosis in the tumor cell lines that overexpressed NOTCH3 but not in those with minimal amounts of NOTCH3 expression (Park J T et al., Cancer Res. 66: 6312-8 (2006)). Furthermore, a phase I clinical trial for a NOTCH inhibitor, MK0752 (developed by Merck, Whitehouse Station, N.J.), has been launched for relapsed or refractory T-ALL patients and advanced breast cancers.

Anti-NOTCH antibodies can also act as NOTCH antagonists by binding to NOTCH receptors and blocking their binding to a NOTCH ligand. Anti-NOTCH agents can also encompass antibodies that specifically bind to NOTCH ligands (anti-NOTCH ligand antibodies). The NOTCH receptor/ligand antibodies of the invention can be prepared by any conventional means known in the art.

In certain embodiments, the anti-NOTCH antibody is a monoclonal antibody. Monoclonal antibodies can be prepared by any means known in the art (Goding, Monoclonal Antibodies: Principles and Practice, Academic Press, 1986). Monoclonal antibodies can be prepared using hybridoma methods, such as those described by Kohler and Milstein (1975) Nature 256:495. Monoclonal antibodies can also be made using recombinant DNA methods as described in U.S. Pat. No. 4,816,567. Recombinant monoclonal antibodies or fragments thereof of the desired species can also be isolated from phage display libraries expressing CDRs of the desired species using techniques known in the art (McCafferty et al., Nature, 348:552-554 (1990); Clackson et al., Nature, 352:624-628 (1991); and Marks et al., J. Mol. Biol., 222:581-597 (1991)).

In some embodiments, the anti-NOTCH antibody is a humanized antibody. Humanized antibodies are antibodies that contain minimal sequences from non-human (e.g., murine) antibodies within the variable regions. Such antibodies are used therapeutically to reduce antigenicity and HAMA (human anti-mouse antibody) responses when administered to a human subject. Humanized antibodies can be produced using various techniques known in the art (Jones et al., Nature, 321:522-525 (1986); Riechmann et al., Nature, 332:323-327 (1988); Verhoeyen et al., Science, 239:1534-1536 (1988)). An antibody can be humanized by substituting the CDRs of a human antibody with that of a non-human antibody (e.g., mouse, rat, rabbit, hamster, etc.) having the desired specificity, affinity, and/or capability. The humanized antibody can be further modified by the substitution of additional residues either in the variable human framework region and/or within the replaced non-human residues to refine and optimize antibody specificity, affinity, and/or capability.

In other embodiments, the anti-NOTCH antibody is a fully human antibody. Human antibodies can be prepared using various techniques known in the art Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boerner et al., 1991, J. Immunol, 147 (1):86-95; and U.S. Pat. No. 5,750,373). Also, the human antibody can be selected from a phage library, where that phage library expresses human antibodies (Vaughan et al., 1996, Nat. Biotech., 14:309-314; Sheets et al., 1998, Proc. Nat'l. Acad. Sci., 95:6157-6162; Hoogenboom and Winter, 1991, J. Mol. Biol., 227:381; Marks et al., 1991, J. Mol. Biol., 222:581). Human antibodies can also be made in transgenic mice containing human immunoglobulin loci that are capable upon immunization of producing a broad repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In one embodiment, the anti-NOTCH antibody specifically binds the ligand binding region of the NOTCH receptors. In other embodiments, the anti-NOTCH antibody binds to one or more EGF-like domains that do not encompass the ligand binding region of the NOTCH receptors. In another embodiment, the anti-NOTCH antibody specifically binds to an epitope in the LNR-HD negative regulatory region of the NOTCH receptors. In another embodiment, the anti-NOTCH antibody blocks cleavage of the NOTCH receptor.

In certain embodiments, the anti-NOTCH agents are antibodies that specifically bind NOTCH ligands, including delta-like ligands and Jagged proteins. In one embodiment, the anti-NOTCH agents are antibodies that bind delta-like ligand 4 (DLL4). In one embodiment, the anti-NOTCH agents are antibodies that bind Jagged 1 or 2.

In certain embodiments, the anti-NOTCH antibody is the antibody produced by the hybridoma deposited with ATCC on Aug. 7, 2008 and having ATCC deposit number PTA-9405, also known as “murine 52M51.” The murine 52M51 antibody is described in detail in International Patent Application PCT/US2009/003995, filed Jul. 8, 2009 and published as WO 2010/005567, incorporated herein by reference in its entirety.

In certain embodiments, the anti-NOTCH antibody is a humanized anti-NOTCH antibody that comprises a heavy chain variable region comprising CDR amino acid sequences CDR1 (SEQ ID NO: 5); CDR2 (SEQ ID NO: 6); and CDR3 (SEQ ID NO: 7); and a light chain variable region comprising CDR amino acid sequences CDR1 (SEQ ID NO: 8); CDR2 (SEQ ID NO: 9); and CDR3 (SEQ ID NO: 10). In one embodiment, the humanized anti-NOTCH antibody comprises the heavy chain variable region sequence of SEQ ID NO:14 and the light chain variable region sequence of SEQ ID NO: 18.

In certain embodiments, the anti-NOTCH antibody is the OMP-52M51 humanized antibody encoded by the plasmid deposited with ATCC on Oct. 15, 2008, having ATCC deposit number PTA-9549, also known as “52M51 H4L3.” The terms “52M51 H4L3,” “OMP-52M51,” and “52M51” are typically used interchangeably herein. 52M51 H4L3 is also described in detail in International Patent Application PCT/US2009/003995, filed Jul. 8, 2009 and published as WO 2010/005567, and U.S. Patent Publication No. 2011/0311552, both of which are incorporated by reference herein in their entirety. OMP-52M51 comprises the heavy chain variable region sequence of SEQ ID NO:14 and the light chain variable region sequence of SEQ ID NO: 18.

In certain embodiments, the anti-NOTCH antibody is an antibody that competes with the antibody OMP-52M51 for specific binding to human NOTCH1.

In certain embodiments, the anti-NOTCH antibody is the antibody produced by the hybridoma deposited with ATCC on Jul. 6, 2009 and having ATCC deposit number PTA-10170, also known as 59R5. The 59R5 antibody is described in detail in; U.S. patent application Ser. No. 12/499,627, filed Jul. 8, 2009 and published as U.S. Patent Application Pub. No. 2010/0111958, incorporated herein by reference in its entirety.

The anti-NOTCH antibody 59R5 comprises a heavy chain variable region comprising CDR amino acid sequences CDR1 (SEQ ID NO: 23); CDR2 (SEQ ID NO: 24); and CDR3 (SEQ ID NO: 25); and a light chain variable region comprising CDR amino acid sequences CDR1 (SEQ ID NO: 26); CDR2 (SEQ ID NO: 27); and CDR3 (SEQ ID NO: 28). In one embodiment, the 59R5 antibody comprises the heavy chain variable region sequence of SEQ ID NO:30 and the light chain variable region sequence of SEQ ID NO: 32.

In certain embodiments, the anti-NOTCH antibody is an antibody that competes with the antibody 59R5 for specific binding to human NOTCH2 or NOTCH3.

Other anti-NOTCH antibodies are known in the art. Anti-NOTCH antibodies are available from commercial sources (for example, Santa Cruz Biotechnology, Inc. catalog no. sc-6014 is a goat polyclonal antibody that binds to the extracellular domain of human NOTCH1). In some embodiments, the NOTCH antagonist can be one of the anti-NOTCH antibodies described in U.S. Pat. No. 7,919,092, filed May 31, 2008; U.S. patent application Ser. No. 12/010,421, filed Jan. 24, 2008 and published as U.S. Patent Application Pub. No. 2009/0047285; International Application No. PCT/US2011/021135, filed Jan. 13, 2011 and published as International Application Pub. No. WO 2011/088215; U.S. patent application Ser. No. 12/156,590, filed Jun. 3, 2008, and published as U.S. Patent Application Pub. No. 2009/0258026; U.S. patent application Ser. No. 11/958,099, filed Dec. 17, 2007, and published as U.S. Patent Application Pub. No. 2008/0226621.

### V. Use of Mutated NOTCH Polypeptides in Screening Assays

The methods of the present invention have other applications as well. For example, the mutated NOTCH polypeptides can be used to screen for compounds that modulate the expression of NOTCH in vitro or in vivo, which compounds in turn can be useful in treating or preventing cancer in patients. In another example, the mutated NOTCH polyepeptides can be used to monitor the response to treatments for cancer.

This disclosure further relates in some embodiments to novel methods for screening test compounds for their ability to treat, detect, analyze, ameliorate, reverse, and/or prevent neoplasia, especially pre-cancerous lesions. In particular, the present disclosure provides methods for identifying test compounds that can be used to treat, ameliorate, reverse, and/or prevent neoplasia, including precancerous lesions. The compounds of interest can be tested by exposing the novel activating NOTCH variants described herein to the compounds, and if a compound inhibits one of the NOTCH variants, the compound is then further evaluated for its anti-neoplastic properties. These compounds can include, but are not limited to, small molecule inhibitors, nucleic acids, and antibodies. One aspect involves a screening method to identify a compound effective for treating, preventing, or ameliorating neoplasia, which method includes determining whether the compound inhibits the growth of NOTCH variant tumor cells in a xenograft model.

In a related embodiment, the ability of a test compound to inhibit the activity of one or more of the mutated NOTCH polypeptides of Table 1 can be measured. One of skill in the art will recognize that the techniques used to measure the activity of a particular NOTCH mutant biomarker will vary depending on the function and properties of the mutant.

Test compounds capable of modulating the activity of any of the mutated NOTCH polypeptides of Table 1 can be administered to patients who are suffering from or are at risk of developing cancer.

### VI. Treatment Methods

Anti-NOTCH therapeutic agents, such as gamma-secretase inhibitors and anti-NOTCH receptor/ligand antibodies can be used to treat cancer cells in cases in which uncontrolled growth is associated with the NOTCH receptor mutations described herein. The anti-NOTCH therapeutic agents can also be used to treat cancer cells comprising NOTCH ICD above a predetermined standard as described herein. In certain embodiments, the anti-NOTCH agents are useful in inhibiting tumor growth, inducing differentiation, and/or reducing tumor volume. In addition, the invention provides a method of reducing the tumorigenicity of a tumor in a subject, comprising administering a therapeutically effective amount of an anti-NOTCH agent to a subject having NOTCH activating mutations and/or having activated NOTCH. In one embodiment, tumor cells comprise an activating NOTCH mutation. In another embodiment, tumor cells comprise activated NOTCH. Tumor cell NOTCH can be activated through various mechanisms. For example, tumor cell NOTCH may be activated because of the tumor cell comprises a mutation in a NOTCH regulator, for example, but not limited to, FBW7. In another non-limiting example, NOTCH activation may be due to loss of NUMB expression. In certain embodiments, the tumor comprises cancer stem cells. In certain embodiments, the frequency of cancer stem cells in the tumor is reduced by administration of the anti-NOTCH agent.

In one embodiment, anti-NOTCH therapeutic agents can be used to treat tumors in which at least about 1%, at least about 2%, at least about 3%, at least about 5%, at least about 10%, at least about 25%, or at least about 50% of the tumor cells comprise an activating mutation in a NOTCH receptor. In another embodiment, the anti-NOTCH agents can be used to treat tumors in which at least about 0.1%, at least about 1%, at least about 2%, or at least about 5%, of the tumor cells comprise an activating mutation of the invention.

In another embodiment, anti-NOTCH therapeutic agents can be used to treat tumors in which at least about 1%, at least about 2%, at least about 3%, at least about 5%, at least about 10%, at least about 25%, or at least about 50% of the tumor cells nuclei a comprise weak, intermediate or strong NOTCH ICD specific IHC staining. In a further embodiment, the anti-NOTCH agents can be used to treat tumors characterized by an anti-NOTCH ICD H-score of at least about 10, at least about 20, at least about 30, at least about 40, at least about 50 or at least about 100 using an immunohistochemical staining procedure as described herein. In another embodiment, the anti-NOTCH agents can be used to treat tumors characterized by an anti-NOTCH ICD H-score of more than about 10, more than about 20, more than about 30, more than about 40, more than about 50 or more than about 100 using an immunohistochemical staining procedure as described herein.

In certain embodiments, the cancer that is treated with an anti-NOTCH therapeutic agent is a solid tumor selected from the group consisting of: lung cancer, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, breast cancer, colon cancer, melanoma, biliary tract cancer, and head and neck cancer. In a further embodiment, the cancer is breast cancer. In one embodiment, the anti-NOTCH therapeutic agents can be used to treat triple negative (estrogen receptor (ER−), progesterone receptor (PR−), and HER2 (HER2−)) breast cancer cells (TNBC). In a further embodiment, the cancer is small cell cancer. In still further embodiments, the cancer is a small cell lung cancer, gastric cancer, esophageal cancer, hepatocellular carcinoma, or cholangiocarcinoma. In another embodiment, the cancer is a biliary tract cancer.

In one embodiment, the anti-NOTCH therapeutic agents are particularly useful in treating patients that have already undergone some form of treatment. In another embodiment, the anti-NOTCH agents are used to treat a patient that previously failed with a cancer therapy. Failed cancer therapies can include, but are not limited to, chemotherapy, adjuvant therapy, neoadjuvant therapy, and combinations thereof. In one embodiment, the anti-NOTCH agents are used to treat chemotherapy resistant tumors. In another embodiment, the anti-NOTCH agents are used to treat chemotherapy resistant breast cancer. In another embodiment, the anti-NOTCH agents are used to treat chemotherapy resistant TNBC. In some embodiments the anti-NOTCH agents are used to treat breast cancer, small cell lung cancer, gastric cancer, esophageal cancer, hepatocellular carcinoma, or cholangiocarcinoma in a patient that has failed a previous cancer therapy.

In one embodiment, the treatment method involves first testing a biological sample containing cancer cells removed from a patient to determine whether a mutated form of a NOTCH receptor is present or whether they comprise a level of NOTCH ICD above a predetermined standard. Patients whose samples evidence the presence of mutations or elevated NOTCH ICD would then be treated using a NOTCH inhibitor that interferes with NOTCH receptor activity. The dosage administered will depend upon the particular condition being treated, the route of administration and clinical considerations that are well known in the art. Dosages can be gradually increased until a beneficial effect, e.g., a slowing of tumor growth, is detected. The NOTCH inhibitors can then be provided in either single or multiple dosage regimens and can be given either alone or in conjunction with other therapeutic agents.

Treatment of mutant NOTCH receptor-associated cancers is compatible with any route of administration and dosage form. Depending upon the particular condition being treated, certain dosage forms will tend to be more convenient or effective than others. For example, topical administration may be preferred in treating skin cancers, whereas parenteral administration might be preferred for solid tumors. Apart from parenteral and topical preparations, agents may be administered orally, perorally, internally, intranasally, rectally, vaginally, lingually and transdermally. Specific dosage forms include tablets, pills, capsules, powders, aerosols, suppositories, skin patches, parenterals and oral liquids including suspensions, solutions and emulsions. Sustained release dosage forms may also be used. All dosage forms can be prepared using methods that are standard in the art (see, e.g., Remington's Pharmaceutical Sciences, 16th ed., Easton, Pa. (1980)).

In certain embodiments, the administration of an anti-NOTCH therapeutic agent (e.g., anti-NOTCH antibody) may be by intravenous injection or intravenously. In some embodiments, the administration is by intravenous infusion. In some embodiments, the administration of the anti-NOTCH agent may be by a non-intravenous route.

The appropriate dosage of an anti-NOTCH antibody or other anti-NOTCH therapeutic agent depends on the type of disease to be treated, the severity and course of the disease, the responsiveness of the disease, whether the antibody or agent is administered for therapeutic or preventative purposes, previous therapy, patient's clinical history, and so on all at the discretion of the treating physician. The antibody or other agent can be administered one time or over a series of treatments lasting from several days to several months, or until a cure is effected or a diminution of the disease state is achieved (e.g. reduction in tumor size). Optimal dosing schedules can be calculated from measurements of drug accumulation in the body of the patient and will vary depending on the relative potency of an individual antagonist. The administering physician can easily determine optimum dosages, dosing methodologies and repetition rates. In general, dosage is from 0.01 μg to 100 mg per kg of body weight, and can be given once or more daily, weekly, monthly or yearly. The treating physician can estimate repetition rates for dosing based on measured residence times and concentrations of the antibody or agent in bodily fluids or tissues.

As is known by those of skill in the art, doses used will vary depending on the clinical goals to be achieved. In some embodiments, each dose of the anti-NOTCH antibody is about 0.25 mg/kg to about 15 mg/kg. In some embodiments, each dose is about 0.25, 0.5, 1, 2, 3, 4, 5, 6, 7, 8, 9, 10, 11, 12, 13, 14, 15, 16, 17, 18, 19, or 20 mg/kg. In certain embodiments, each dose is about 0.5 mg/kg. In certain embodiments, each dose is about 1 mg/kg. In certain embodiments, each dose is about 2.5 mg/kg. In certain embodiments, each dose is about 5 mg/kg. In certain embodiments, each dose is about 7.5 mg/kg. In certain embodiments, each dose is about 10 mg/kg. In certain embodiments, each dose is about 12.5 mg/kg. In certain embodiments, each dose is about 15 mg/kg.

In certain embodiments, the anti-NOTCH antibody or other anti-NOTCH therapeutic agent used in the methods described herein is administered to the patient using an intermittent dosing regimen, which may in some instances reduce side effects and/or toxicities associated with administration of the anti-NOTCH antibody or agent. As used herein, “intermittent dosing” refers to a dosing regimen using a dosing interval of more than once a week, e.g., dosing once every 2 weeks, once every 3 weeks, once every 4 weeks, etc. In some embodiments, a method for treating a cancer in a human patient comprises administering to the patient an effective dose of an anti-NOTCH antibody or agent according to an intermittent dosing regimen. In some embodiments, a method for treating a cancer in a human patient comprises administering to the patient an effective dose of an anti-NOTCH antibody or agent according to an intermittent dosing regimen, and increasing the therapeutic index of the anti-NOTCH antibody or agent. In some embodiments, the intermittent dosing regimen comprises administering an initial dose of an anti-NOTCH therapeutic agent to the patient, and administering subsequent doses of the anti-NOTCH therapeutic agent about once every 2 weeks. In some embodiments, the intermittent dosing regimen comprises administering an initial dose of an anti-NOTCH therapeutic agent to the patient, and administering subsequent doses of the anti-NOTCH therapeutic agent about once every 3 weeks. In some embodiments, the intermittent dosing regimen comprises administering an initial dose of an anti-NOTCH therapeutic agent to the patient, and administering subsequent doses of the anti-NOTCH therapeutic agent about once every 4 weeks.

In certain embodiments, the anti-NOTCH antibody used in the methods is OMP-52M51, or an antibody comprising the six CDRs and/or the variable regions of OMP-52M51, and the antibody is administered to subjects intravenously at a dosage of about 0.25 mg/kg to about 10 mg/kg approximately every three weeks.

In some alternative embodiments, the anti-NOTCH antibody used in the methods is OMP-59R5, or an antibody comprising the six CDRs and/or the variable regions of OMP-59R5, and the antibody is administered to subjects intravenously at a dosage of about 2.5 mg/kg to about 7.5 mg/kg (e.g., about 2.5 mg/kg, about 5 mg/kg, or about 7.5 mg/kg) approximately every two to three weeks.

In certain embodiments, in addition to administering an anti-NOTCH therapeutic agent such as an anti-NOTCH antibody, the method or treatment further comprises administering at least one additional therapeutic agent or therapy. An additional therapeutic agent or therapy can be administered prior to, concurrently with, and/or subsequently to, administration of the anti-NOTCH therapeutic agent. In some embodiments, the at least one additional therapeutic agent or therapy comprises 1, 2, 3, or more additional therapeutic agents or therapies.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects. Combination therapy may decrease the likelihood that resistant cancer cells will develop.

It will be appreciated that the combination of an anti-NOTCH therapeutic agent and an additional therapeutic agent or therapy may be administered in any order or concurrently. In some embodiments, the anti-NOTCH therapeutic agent will be administered to patients that have previously undergone treatment with a second therapeutic agent or therapy. In certain other embodiments, the anti-NOTCH therapeutic agent and a second therapeutic agent or therapy will be administered substantially simultaneously or concurrently. For example, a subject may be given the anti-NOTCH therapeutic agent while undergoing a course of treatment with a second therapeutic agent (e.g., chemotherapy). In certain embodiments, the anti-NOTCH therapeutic agent will be administered within 1 year of the treatment with a second therapeutic agent. In certain alternative embodiments, the anti-NOTCH therapeutic agent will be administered within 10, 8, 6, 4, or 2 months of any treatment with a second therapeutic agent. In certain other embodiments, the anti-NOTCH therapeutic agent will be administered within 4, 3, 2, or 1 weeks of any treatment with a second therapeutic agent. In some embodiments, the anti-NOTCH therapeutic agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

In certain embodiments, the anti-NOTCH therapeutic agent is administered to the patient using an intermittent dosing regimen, which may in some instances reduce side effects and/or toxicities associated with administration of the anti-NOTCH therapeutic agent. As used herein, “intermittent dosing” refers to a dosing regimen using a dosing interval of more than once a week, e.g., dosing once every 2 weeks, once every 3 weeks, once every 4 weeks, etc. In some embodiments, a method for treating a cancer in a human patient comprises administering to the patient an effective dose of an anti-NOTCH therapeutic agent according to an intermittent dosing regimen. In some embodiments, a method for treating a cancer in a human patient comprises administering to the patient an effective dose of an anti-NOTCH therapeutic agent according to an intermittent dosing regimen, and increasing the therapeutic index of the anti-NOTCH therapeutic agent. In some embodiments, the intermittent dosing regimen comprises administering an initial dose of an anti-NOTCH therapeutic agent to the patient, and administering subsequent doses of the anti-NOTCH therapeutic agent about once every 2 weeks. In some embodiments, the intermittent dosing regimen comprises administering an initial dose of an anti-NOTCH therapeutic agent to the patient, and administering subsequent doses of the anti-NOTCH therapeutic agent about once every 3 weeks. In some embodiments, the intermittent dosing regimen comprises administering an initial dose of an anti-NOTCH therapeutic agent to the patient, and administering subsequent doses of the anti-NOTCH therapeutic agent about once every 4 weeks.

As is known by those of skill in the art, doses used will vary depending on the clinical goals to be achieved. In some embodiments, each dose of an anti-NOTCH antibody is about 0.25 mg/kg to about 15 mg/kg. In some embodiments, each dose is about 0.25, 0.5, 1, 2, 3, 4, 5, 6, 7, 8, 9, 10, 11, 12, 13, 14, 15, 16, 17, 18, 19, or 20 mg/kg. In certain embodiments, each dose is about 0.5 mg/kg. In certain embodiments, each dose is about 1 mg/kg. In certain embodiments, each dose is about 2.5 mg/kg. In certain embodiments, each dose is about 5 mg/kg. In certain embodiments, each dose is about 7.5 mg/kg. In certain embodiments, each dose is about 10 mg/kg. In certain embodiments, each dose is about 12.5 mg/kg. In certain embodiments, each dose is about 15 mg/kg.

Therapeutic agents used for the treatment of cancers include, but are not limited to, antibiotics such as daunorubicin, doxorubicin, mitoxantrone and idarubicin; topoisomerase inhibitors such as etoposide, teniposide, and topotecan; DNA synthesis inhibitors such as carboplatin; DNA-damaging agents such as cyclophosphamide, bendamustine, chlorambucil, procarbazine, dacarbazine, and ifosfamide; cytotoxic enzymes such as asparaginase and pegaspargase; tyrosine kinases inhibitors such as imatinib mesylate, dasatinib, ponatinib, and nilotinib; antimetabolites such as azacitidine, clofarabine, cytarabine, cladribine, fludarabine, hydroxyurea, mercaptopurine, methotrexate, thioguanine, pralatrexate, and nelarabine; synthetic hormones such as prednisone, prednisolone and dexamethasone; antimitotic agents such as vincristine and vinblastine; monoclonal antibodies such as rituximab (e.g., RITUXAN), alemtuzumab, and ofatumumab; radioimmunotherapy agents such as Iodine I 131 tositumomab (e.g., BEXXAR) or ibritumomab tiuxetan (e.g., ZEVALIN); mTor inhibitors such as temsirolimus; histone deacetylase inhibitors such as vorinostat and romidepsin; hematopoietic stem cell mobilizers such as plerixafor; cytotoxic recombinant proteins such as denileukin difitox; protein synthesis inhibitors such as omacetaxine; immunomodulatory drugs such as thalidomide and lenalidomide; cyclin-dependent kinase inhibitors such as flavopiridol; and proteasome inhibitors such as bortezomib (e.g., VELCADE); as well as combinations thereof.

Therapeutic agents that may be administered in combination with the anti-NOTCH therapeutic agents include the above name therapeutic agents as well as other chemotherapeutic agents. Thus, in some embodiments, the method or treatment involves the combined administration of an anti-NOTCH therapeutic agent and a chemotherapeutic agent or cocktail of multiple different chemotherapeutic agents. Treatment with an anti-NOTCH therapeutic agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service Editor M. C. Perry, Williams & Wilkins, Baltimore, Md. (1992).

Chemotherapeutic agents useful in the instant invention include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof.

In certain embodiments, the treatment involves the combined administration of an anti-NOTCH therapeutic agent described herein and radiation therapy. Treatment with the anti-NOTCH therapeutic agent can occur prior to, concurrently with, or subsequent to administration of radiation therapy. Dosing schedules for such radiation therapy can be determined by the skilled medical practitioner. In some embodiments, the anti-NOTCH antibody or other anti-NOTCH therapeutic agent is administered after radiation treatment. In some embodiments, the anti-NOTCH antibody or other anti-NOTCH therapeutic agent is administered with radiation therapy.

In some embodiments, a second therapeutic agent comprises an antibody. Thus, treatment can involve the combined administration of an anti-NOTCH therapeutic agent of the present invention with other antibodies against additional tumor-associated antigens including, but not limited to, antibodies that bind to EGFR, ErbB2, DLL4, or NF-κB. Exemplary anti-DLL4 antibodies are described, for example, in U.S. Pat. No. 7,750,124. Additional anti-DLL4 antibodies are described in, e.g., International Patent Pub. Nos. WO 2008/091222 and WO 2008/0793326, and U.S. Patent Application Pub. Nos. 2008/0014196; 2008/0175847; 2008/0181899; and 2008/0107648. Combined administration can include co-administration, either in a single pharmaceutical formulation or using separate formulations, or consecutive administration in either order but generally within a time period such that all active agents can exert their biological activities simultaneously.

Furthermore, treatment with the anti-NOTCH therapeutic agents described herein can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician.

### VII. Kits

Kits for practicing the methods of the invention are further provided. By “kit” is intended any manufacture (e.g., a package or a container) comprising at least one reagent, e.g., an antibody, a nucleic acid probe, etc. for specifically detecting a mutated NOTCH receptor of the invention, or for specifically detecting NOTCH ICD. The kit can be promoted, distributed, or sold as a unit for performing the methods of the present invention. Additionally, the kits may contain a package insert describing the kit and including instructional material for its use.

In one embodiment, kits for practicing the methods of the invention are provided. Such kits are compatible with both manual and automated screening. For immunoassay analysis, the kits comprise at least one antibody directed to a mutated NOTCH receptor or at least one antibody directed to NOTCH ICD and chemicals for the detection of antibody binding to the mutated NOTCH receptor or NOTCH ICD. Any chemicals that detect antigen-antibody binding may be used in the practice of the invention. In some embodiments, the detection chemicals comprise a labeled polymer conjugated to a secondary antibody. For example, a secondary antibody that is conjugated to an enzyme that catalyzes the deposition of a chromogen at the antigen-antibody binding site may be provided. Such enzymes and techniques for using them in the detection of antibody binding are well known in the art. In one embodiment, the kit comprises a secondary antibody that is conjugated to an HRP-labeled polymer. Chromogens compatible with the conjugated enzyme (e.g., DAB in the case of an HRP-labeled secondary antibody) and solutions, such as hydrogen peroxide, for blocking non-specific staining may be further provided.

The kits of the present invention may further comprise a peroxidase blocking reagent (e.g., hydrogen peroxide) and a protein blocking reagent (e.g., purified casein).

Additionally, the kits may comprise reagents for practicing the methods of the invention using microarrays, such as microbeads on a solid support, or by nucleic acid amplification, including oligonucleotide primers and a DNA polymerase.

Positive and/or negative controls can be included in the kits to validate the activity and correct usage of reagents employed in accordance with the invention. Controls can include samples, such as tissue sections, cells fixed on glass slides, etc., known to be either positive or negative for the presence of the NOTCH mutation or NOTCH ICD of interest, or other samples comprising the mutated NOTCH receptors or NOTCH ICD. The design and use of controls is standard and well within the routine capabilities of those in the art.

It will be further appreciated that any or all steps in the methods of the invention could be implemented by personnel or, alternatively, performed in an automated fashion. Thus, the steps of body sample preparation, sample staining, and detection of the NOTCH mutations or NOTCH ICD can be automated.

Embodiments of the present disclosure can be further defined by reference to the following examples. It will be apparent to those skilled in the art that many modifications, both to materials and methods, can be practiced without departing from the scope of the present disclosure.

### EXAMPLES

### Example 1

### Identification of the OMP-B40 and OMP-B37 NOTCH Mutations

Low passaged xenograft tumors derived from human primary tumors were harvested, minced, and digested using collagenase and trypsin in HBSS medium as previously described (Dylla et al, PLoS ONE. 2008, 36:e2428). To deplete the mouse cells, the freshly prepared single cells were incubated with biotinylated anti-mouse H-2K^{d} (clone SF1-1.1, Biolegend) and anti-mouse CD45 (30-F11, Biolegend) on ice. The unbound antibodies were removed by washing twice with FACS buffer FACS buffer (lx Hanks Buffered Saline Solution (HBSS), 2% heat-inactivated Fetal Calf Serum and 25 mM HEPES pH 7.4). Streptavidin magnetic beads (88817; Pierce) were then added to the single suspension and incubated at 4° C. The unbound human tumor cells were collected and the total genomic DNA was extracted from the purified tumor cells using the Bioneer AccuPrep Genomic DNA Extraction Kit (Bioneer CA). The DNA was amplified and purified using Qiagen Repli-G whole genome amplification kit based on the manufacturer's protocol (Qiagen CA).

Sequencing of Exon 26 (432nt), 32 (148 nt), and 34 (1488 nt) of NOTCH1 and Exon 33 (1053 nt) of NOTCH3 was performed using the 3730x1 DNA sequencer (Applied Biosystems). The sequencing results were aligned to RefSeq nucleotide sequences of NOTCH1 (NM_{—}017617.3), NOTCH2 (NM_{—}024408.2), and NOTCH3 (NM_{—}000435.2). Mutations were identified using Mutation Surveyor (SoftGenetics PA) and Sequencher (GeneCods MI) software.

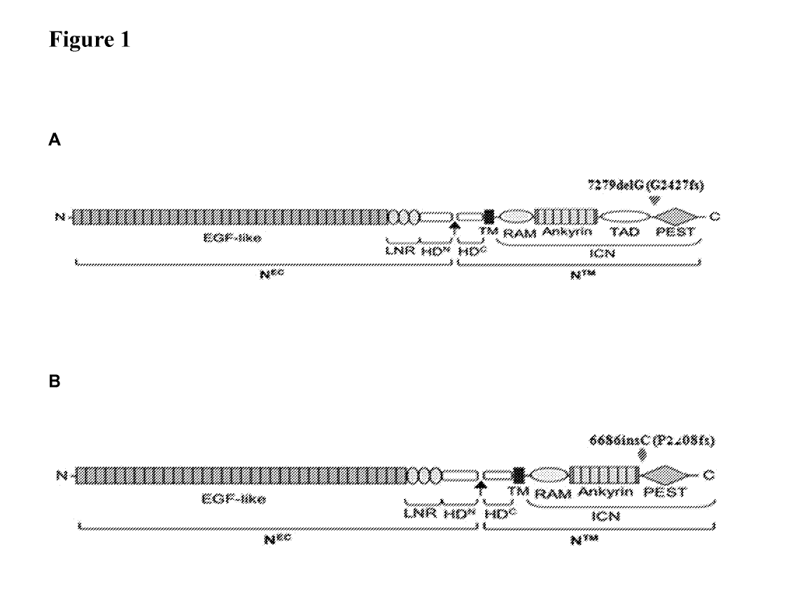

In the human NOTCH1 gene, the OMP-B40 mutation was identified as a heterozygous deletion of guanine (G) at the nucleotide site 7279 (RefSeq NM_{—}017617.3) in the OMP-B40-p2 breast tumor (FIG. 1A). The OMP-B40-p2 breast tumor is a minimally passaged breast tumor sample from a patient whose cancer progressed after treatment with chemotherapy. This breast tumor is triple-negative and is a small cell type of breast tumor. In the sample analyzed, a polymorphic heterozygous insertion of TGG at the nucleotide site 4735 was also identified that is believed to have no functional significance. The G deletion causes a reading frame shift in PEST (G2427fs) domain of NOTCH1. The frameshift mutation of the PEST domain may stabilize the intracellular domain of NOTCH1 (ICD) and result in the activation of NOTCH pathway. In the human NOTCH3 gene, the OMP-B37 mutation was identified as a homozygous insertion of cytosine (C) at the nucleotide site 6622 (NM_{—}000435.2) in the OMP-B37-p2 breast tumor which causes a reading frame shift at amino acid position 2208 (P2208fs) of the PEST domain (FIG. 1B). The OMP-B37-p2 breast tumor is a minimally passaged, triple negative breast tumor sample. The frameshift mutation of the PEST domain may stabilize the intracellular domain of NOTCH3 (ICD), and thus result in a gain-of-function activation of NOTCH pathway in this tumor.

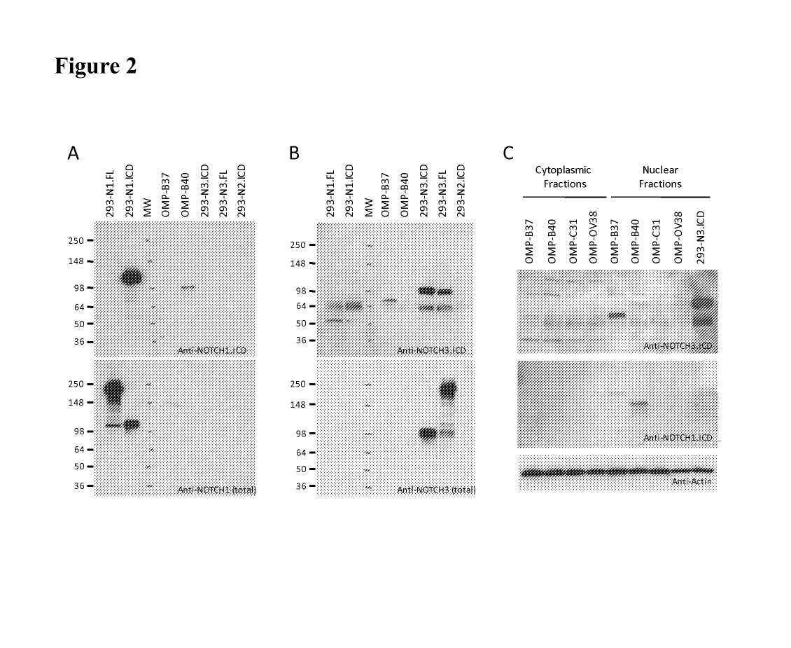

To analyze protein expression and localization, 293T cells were transiently transfected with a control vector, a NOTCH1.ICD-, NOTCH2.ICD-, NOTCH3.ICD-, NOTCH1.Full Length (FL)-, or NOTCH3.FL-expression plasmid. Nuclear and cytoplasmic fractions of transfected cells and xenograft tumors were prepared using NE-PER Nuclear and Cytoplasmic Extraction Kit (Thermo Scientific), separated on 4-12% Tris-Glycine gels, and transferred to PVDF membranes. 100 μg total protein was loaded per lane. Antibodies against the cleaved forms of NOTCH1 intracellular domain (ICD) and NOTCH3 ICD were generated by immunizing rabbits with a NOTCH1 peptide (VLLSRKRRRQHGQLW; SEQ ID NO:36) and a NOTCH3 peptide (VMVARRKREHSTLW; SEQ ID NO:37), respectively. Western Blots were probed with rabbit anti-NOTCH1.ICD (60 ng/ml), anti-NOTCH3.ICD (400 ng/ml), anti-total NOTCH1 (Cell Signaling Technology, 1:1000), anti-total NOTCH3 (Cell Signaling Technology, 1:1000), and anti-13-Actin (Sigma-Aldrich, 1:5000) antibodies followed by HRP-conjugated anti-rabbit or HRP-conjugated anti-mouse secondary antibodies (Cell Signaling Technology, 1:3000). As shown in FIG. 2, an anti-NOTCH1.ICD antibody reacts specifically against the cleaved NOTCH1 intracellular domain (NOTCH1.ICD) and detects a truncated cleaved NOTCH1 intracellular domain in the nuclear fraction of the OMP-B40 xenograft tumor (FIG. 2A). FIG. 2B shows that an anti-NOTCH3.ICD antibody reacts specifically against the cleaved NOTCH3.ICD and detects a truncated cleaved NOTCH3.ICD in the nuclear fraction of the OMP-B37 xenograft tumor. FIG. 2C shows that truncated NOTCH1 ICD and the truncated NOTCH3 ICD are found predominately in the nuclear fractions of the OMP-B40 and OMP-B37 xenograft tumors. These two tumor lines carry mutations of the PEST domains of NOTCH1 and NOTCH3, respectively. Nuclear NOTCH1 ICD and NOTCH3 ICD are not detected in the OMP-C31 and OMP-OV38 xenograft tumors that carry wild-type NOTCH1, but OMP-C31 contains a 6172insC(het) [P2033fs] mutation in the NOTCH3 gene which is not believed to be an activating mutation (data not shown).

### Example 2

### Activity of NOTCH1 Antagonists in OMP-B40 Tumors

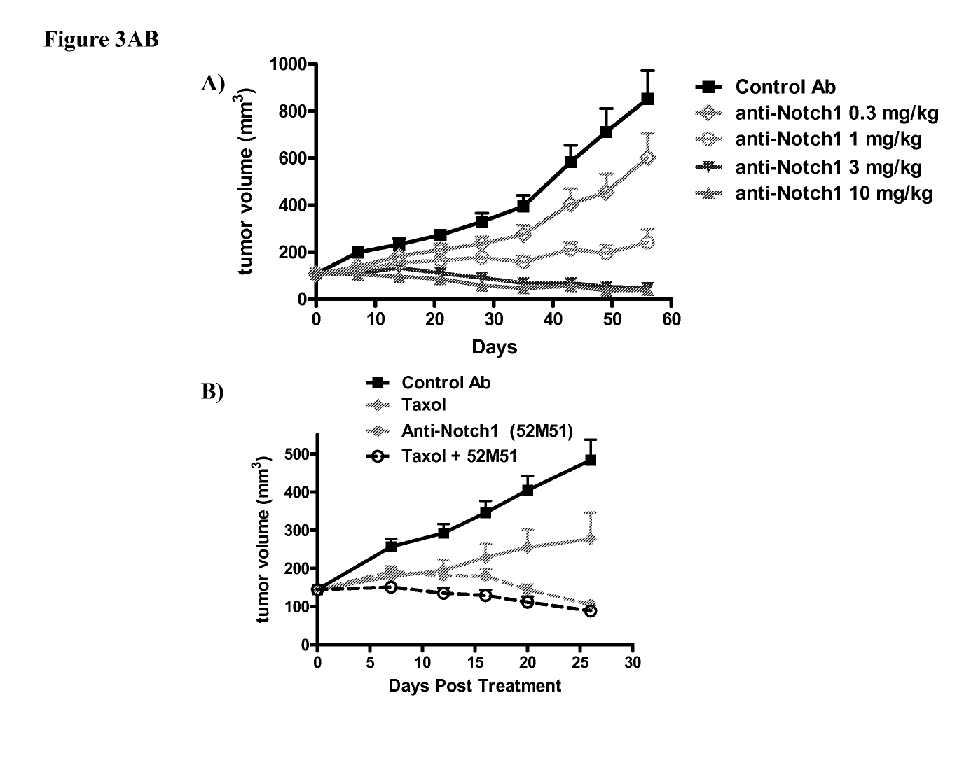

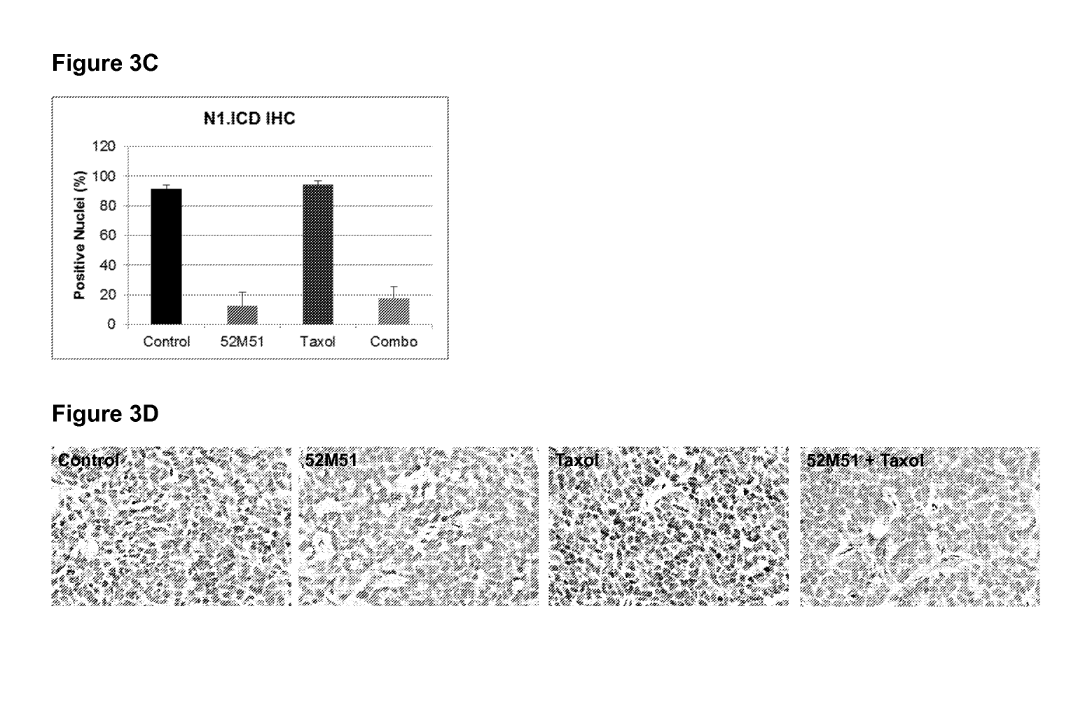

B40 p3 tumor cells were injected subcutaneously into the left flank of 6-7 week old female NOD/SCID mice (300,000 cells/mouse). Mice were monitored weekly and tumors were allowed to grow until they were approximately 140 mm³ (78 days after injection). Mice were randomized into five treatment groups and treated with control antibody, or different doses (0.3 mg/kg, 1 mg/kg, 3 mg/kg, 10 mg/kg) of the OMP-52M51 humanized anti-NOTCH1 antibody. The antibodies were dosed i.p. twice a week. Tumor growth was monitored by caliper measurement once a week. OMP-52M51 treatment resulted in a reduction in tumor volume versus control at all doses tested (FIG. 3A).

Dissociated OMP-B40 p3 tumor cells were re-suspended in injection media (PBS containing 100 ng/mL VEGF and 100 ng/mL bFGF and 0.5× matrigel) and injected subcutaneously into the left flank of 6-7 week old female NOD/SCID mice (300,000 cells/mouse). Mice were monitored weekly and tumors were allowed to grow until they were approximately 140 mm³ (78 days after injection). Mice were randomized into four treatment groups (n=10 mice/group) and treated with control antibody, anti-NOTCH1 antibody OMP-52M51, taxol or a combination of OMP-52M51 with taxol. The antibodies were dosed i.p. twice a week at 15 mg/kg and Taxol was dosed i.p. once a week at 20 mg/kg. Tumor growth was monitored by caliper measurement once a week. The Taxol and OMP-52M51-treated groups resulted in a 57.3% (p<0.03) and 21.8% (p<0.0001) reduction in tumor volume versus control, while the combination group of taxol+52M51-treated mice showed a tumor volume reduction of 18.4% (p<0.0001) versus control and 32.1% (P<0.03) versus taxol alone (FIG. 3B). OMP-52M51-treated groups also displayed a reduction in NOTCH1.ICD as detected by IHC assay, as described in Example 7 below (FIGS. 3C and D). The level of anti-tumor activity demonstrated by OMP-52M51 in the B40 tumor model was a surprisingly high level of activity.

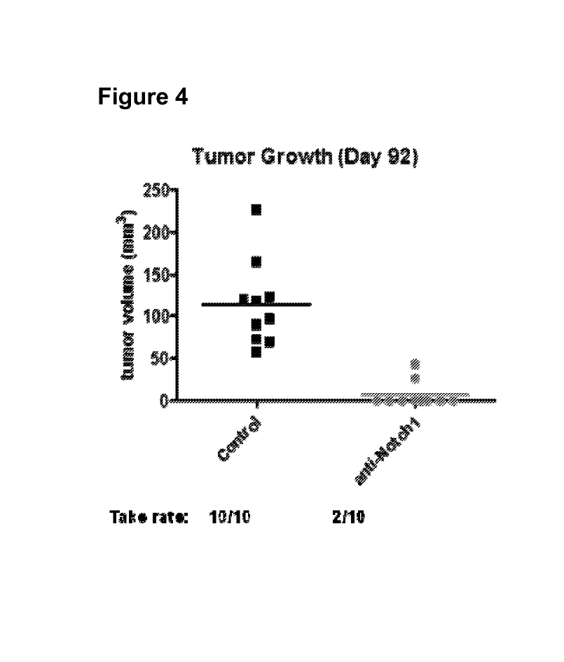

The effect of NOTCH1 treatment on cancer stem cell frequency was also analyzed in OMP-B40 tumors. OMP-B40 breast tumors were treated with a either a control mAb or OMP-52M51 (15 mg/kg twice per week), isolated, dissociated into single cells and the human tumor cells in the xenograft were purified by negative selection with anti-mouse antibodies. One thousand tumor cells from the control and OMP-52M51 treated tumors were injected into 10 mice each. Tumors were allowed to grow for 92 days without further treatment. Tumor volumes on day 92 are shown in FIG. 4 for the control group (black squares) and OMP-52M51 group (gray circles). OMP-B40 tumors grew in each of the 10 mice injected with the control antibody treated cells, whereas only 2 out of 10 mice injected with cells previously treated with OMP-52M51 resulted in detectable tumor growth at day 92, indicating that OMP-52M51 treatment reduced the subsequent tumorigenecity of the cells.

### Example 3

### Activity of NOTCH3 Antagonists in OMP-B37 Tumors

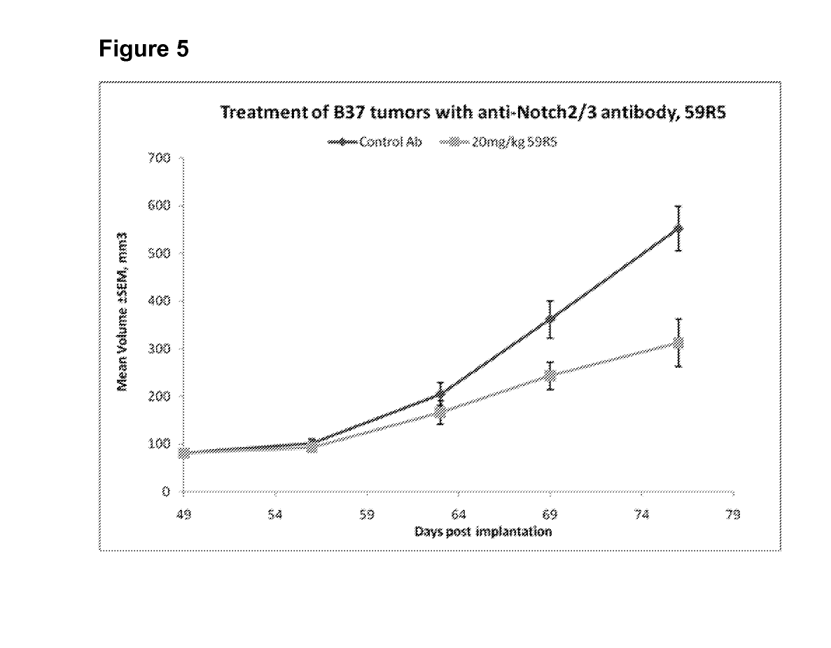

Dissociated lineage depleted OMP-B37 p4 tumor cells were re-suspended in injection media (PBS and 0.5× matrigel) and injected subcutaneously into the right flank of 6-7 week old female NOD/SCID mice (27,000 cells/mouse). Mice were monitored weekly and tumors were allowed to grow until they were approximately 80 mm³ (49 days after injection). Mice were randomized into two treatment groups (n=11 mice/group) and were dosed once a week with 15 mg/kg control antibody, or 20 mg/kg 59R5, an anti-NOTCH2/3 antibody (FIG. 5). Tumor growth was monitored by caliper measurement once a week. Tumor volume reduction of 56.6% (p<0.003) was seen in the 59R5-treated group after 5 weeks of treatment.

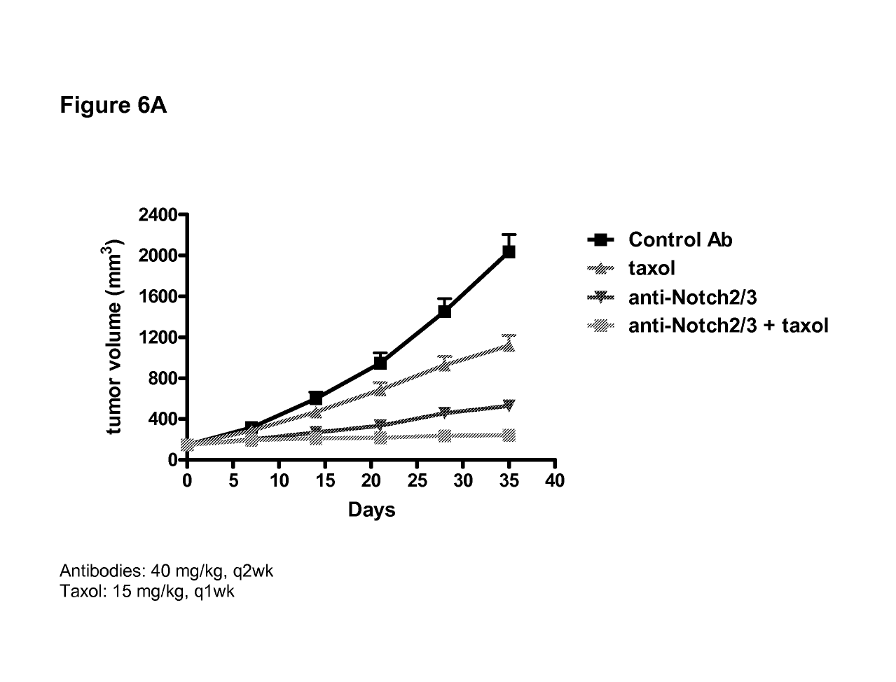

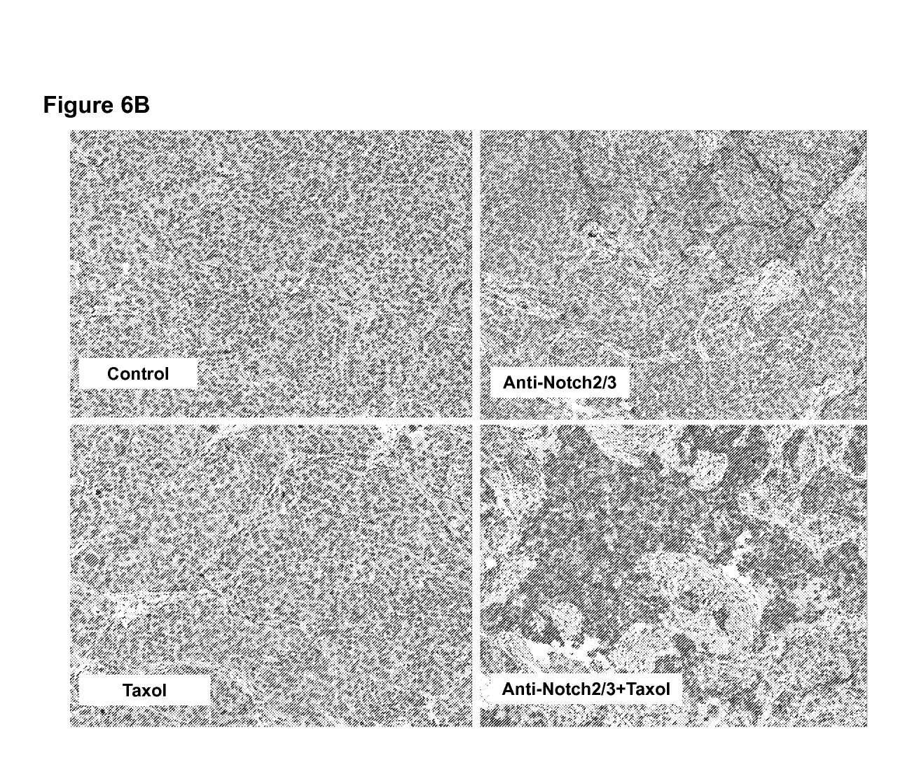

Activity of anti-NOTCH2/3 in combination with Taxol in OMP-B37 breast tumor. The anti-NOTCH2/3 antibody 59R5, taxol, or a combination of 59R5 and taxol were administered to OMP-B37 xenograft mice. Initially, B37 p2 tumor cells were injected subcutaneously into the right flank of 6-7 week old female NOD/SCID mice. Mice were monitored weekly and tumors were allowed to grow until they were approximately 80 mm³ Mice were then administered a control antibody, the 59R5 anti-NOTCH2/3 antibody, taxol or a combination of 59R5 anti-NOTCH2/3 antibody and taxol (FIG. 6). Tumor growth was monitored by caliper measurement once a week. 59R5, either alone or in combination with taxol, greatly decreased tumor volume in xenografted animals (FIG. 6A). 59R5 treatment also increases expression of E-cadherin indicating reversal of Epithelial Mesenchymal Transition (EMT) (FIG. 6B).

### Example 4

### Identification of the OMP-C31 Mutation

Low passaged (p-2) OMP-C31 xenograft tumors derived from human primary colorectal tumors were harvested, minced, and digested using collagenase and trypsin in HBSS medium as previously described (Dylla et al, PLoS ONE. 2008, 36:e2428). To deplete the mouse cells, the freshly prepared single cells were incubated with biotinylated anti-mouse H-2K^{d} (clone SF1-1.1, Biolegend, CA) and anti-mouse CD45 (30-F11, Biolegend, CA) on ice. The unbound antibodies were removed by washing twice with FACS buffer FACS buffer (lx Hanks Buffered Saline Solution (HBSS), 2% heat-inactivated Fetal Calf Serum and 25 mM HEPES pH 7.4). Dynabeads^{@} streptavidin magnetic beads (Invitrogen, CA) were then added to the single suspension and incubated at 4° C. The unbound human tumor cells were collected and the total genomic DNA was extracted from the purified tumor cells using the Bioneer AccuPrep Genomic DNA Extraction Kit (Bioneer, CA). The DNA was amplified and purified using Qiagen Repli-G whole genome amplification Kit based on the manufacturer's protocol (Qiagen, CA).

Sequencing of human NOTCH3 exon25, exon26, and exon 33 (1053 nt) was performed on an ABI 3730x1 DNA Analyzer (Applied Biosystems, CA). Approximately 500 bp amplicon was sequenced using forward and reverse primers.

The sequencing results were aligned to NOTCH3 RefSeq nucleotide sequences (NM_{—}000435.2) with Sequencher v4.10 (Gene Codes, MI). The mutations/InDels were identified using Mutation Surveyor (SoftGenetics, PA) and manually checked in Sequencher software.

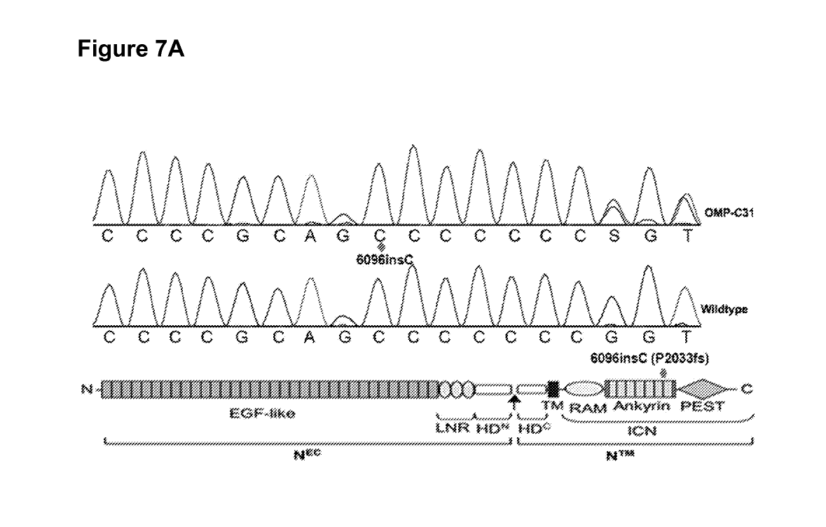

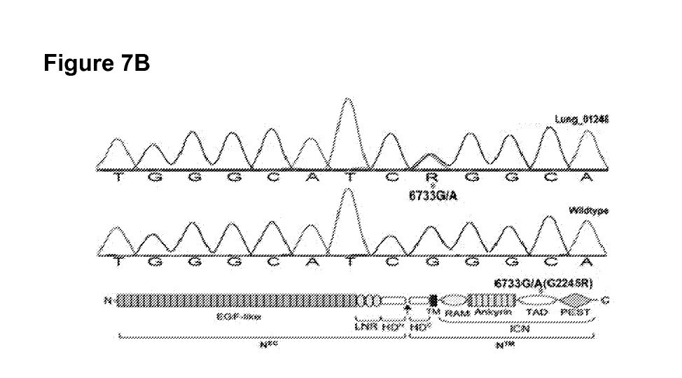

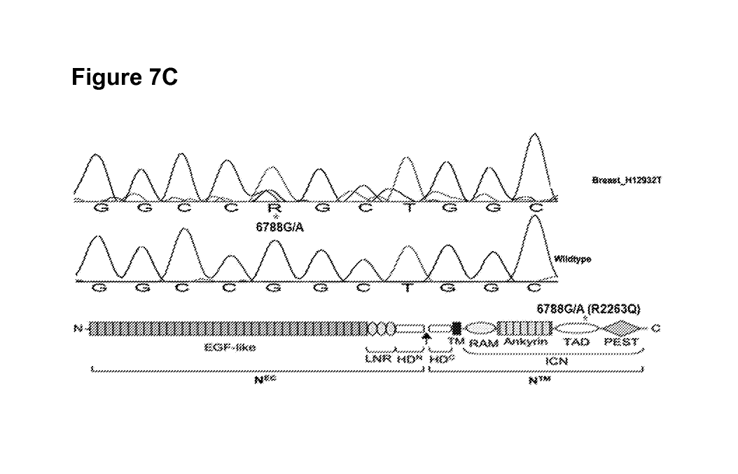

A heterozygous mutation/insertion of cytosine (C) was identified in the OMP-C31-p2 colorectal tumor at the nucleotide site 6096 (NM_{—}000435.2) of the human NOTCH3 gene, which results in a reading frame shift at amino acid position 2033 (P2033fs) in the ANK domain (FIG. 7A). This frameshift mutation is an activating mutation resulting in increased stability of NOTCH3 ICN.

### Example 5

### Identification of the Lung_{—}01246 Mutation

Total DNA samples from 120 human breast tumors were obtained and the DNA was amplified and purified using Qiagen Repli-G whole genome amplification Kit based on the manufacturer's protocol (Qiagen, CA). Sequencing of NOTCH1 exon 26 (432nt) and exon 34 (1488 nt) was performed on an ABI 3730x1 DNA Analyzer (Applied Biosystems, CA). Approximately 500 bp amplicon was sequenced using forward and reverse primers.

The sequencing results were aligned to NOTCH1 RefSeq sequence (NM_{—}017617.3) with Sequencher v4.10 (Gene Codes, MI). The mutations/InDels were identified using Mutation Surveyor (SoftGenetics, PA) and manually checked in Sequencher software.

To detect the mutations possibly occurring in a small subset of tumor cells in a tumor sample, targeted sequencing of NOTCH1 exon 26 and 34 was performed using the Illumina HiSeq2000. Briefly, the genomic regions of the two NOTCH1 exons were PCR amplified by specific PCR primers designed with unique barcodes at their 5′ end. After subjecting the samples to the Qiagen PCR purification kit clean-up, the PCR products were pooled and a paired-end library was constructed using barcoded adaptors according to Illumina protocol (Illumina, CA). 100 bp paired-end sequencing was performed on Illumina HiSeq2000.

After the sequence data were generated, the quality of the data was assessed by the Fastqc program (Babraham Institute, UK). The sequence reads were mapped to the UCSC human genome hg19 assembly using the Burrows-Wheeler Alignment (BWA). After the removal of duplicates reads, the nucleotide variations were called by Samtools (Li et al, 2009, Bioinformatics, 25:2078) and Varscan (Koboldt et al, 2009, Bioinformatics, 25:2283) or GATK (McKenna et al, 2010, Genome Res, 20:1297), and annotated by ANNOVAR (Wang et al, 2010, Nucleic Acids Research, 38:e164).

A heterozygous missense mutation G6733A (p.G2245R, NM_{—}000435.2) was identified in one of the lung tumors (Lung_{—}01246) (FIG. 7B). This mutation resides in NOTCH1 TAD domain. The mutation was reported in T-cell acute lymphoblastic leukemia (Gutierrez et al, 2009, Blood, 114:647). The targeted sequencing by Illumina HiSeq2000 also identified this missense mutation (C->T) in the coding reverse strand from the Integrative Genomics Viewer (IGV).

### Example 6

### Identification of the Breast_H12932T Mutation

Total DNA samples from 80 human breast tumors were obtained and the DNA was amplified and purified using Qiagen Repli-G whole genome amplification Kit based on the manufacturer's protocol (Qiagen, CA). Sequencing of NOTCH1 exon 26 (432nt) and exon 34 (1488 nt) was performed on an ABI 3730x1 DNA Analyzer (Applied Biosystems, CA). Approximately 500 bp amplicon was sequenced using forward and reverse primers.

The sequencing results were aligned to NOTCH1 RefSeq sequence (NM_{—}017617.3) with Sequencher v4.10 (Gene Codes, MI). The mutations/InDels were identified using Mutation Surveyor (SoftGenetics, PA) and manually checked in Sequencher software.

To detect the mutations possibly occurring in a small subset of tumor cells in a tumor sample, targeted sequencing of NOTCH1 exon 26 and 34 was performed using Illumina HiSeq2000. Briefly, the genomic regions of the two NOTCH1 exons were PCR amplified by specific PCR primers designed with unique barcodes at their 5′ end. After subjecting the samples to Qiagen PCR purification kit clean-up, the PCR products were pooled and a paired-end library was constructed using barcoded adaptors according to Illumina's standard protocol (Illumina, CA). 100 bp paired-end sequencing was performed on Illumina HiSeq2000.

After the sequence data was generated, the quality of the data was assessed by the Fastqc program (Babraham Institute, UK). The sequence reads were mapped to the UCSC human genome hg19 assembly using the Burrows-Wheeler Alignment (BWA). After the removal of duplicates reads, the nucleotide variations were called by Samtools (Li et al, 2009, Bioinformatics, 25:2078) and Varscan (Koboldt et al, 2009, Bioinformatics, 25:2283) or GATK (McKenna et al, 2010, Genome Res, 20:1297), and annotated by ANNOVAR (Wang et al, 2010, Nucleic Acids Research, 38:e164).

A heterozygous missense mutation G6788A (p.R2263Q, NM_{—}000435.2) was identified in one of the breast tumors (FIG. 7C). This mutation resides in the NOTCH1 TAD domain. The targeted sequencing by Illumina HiSeq2000 also identified this missense mutation (C->T) in the coding reverse strand from the Integrative Genomics Viewer (IGV).

### Example 7

### Immunohistochemistry of Activated NOTCH 1

Affinity purified NOTCH1.ICD (N1.ICD) rabbit polyclonal antibody was developed which binds the cleavage site of NOTCH1 and specifically detects activated NOTCH1 within the nucleus.

NOTCH1.ICD Immunohistochemistry (IHC) staining was performed with a tyramide signal amplification (TSA) modification of standard IHC protocol. Slides were de-waxed and rehydrated then subjected to antigen retrieval in DAKO TRS solution (DAKO # S1699) under heat and pressure in a BioCare Decloaker benchtop pressure cooker. Endogenous peroxidase was blocked with 6% H2O2 in phosphate buffered saline, then protein block was applied (CAS block, Invitrogen #008120). Primary antibody was incubated overnight at 4° C. at an appropriate dilution. Sections were then incubated with DAKO rabbit HRP polymer (DAKO # K4011), followed by FITC labeled TSA substrate (Perkin Elmer # NEL701001KT). FITC was then detected with HRP-conjugated anti-FITC antibodies (Rockland # RL700-103-096), and DAB substrate (DAKO # K3468) added to visualize the antibody-detection complex.

As described in Example 2, three hundred thousand OMP-B40 tumor cells were injected subcutaneously and allowed to grow to a mean volume of 150 mm³ At that point, 78 days after study start, mice were randomized and treatment started. Mice received either 15 mg/kg control antibody or 15 mg/kg OMP-52M51 humanized antibody twice weekly, with or without 20 mg/kg paclitaxel once weekly, all administered by IP injection. Mean±SEM, n=10 animals per group. 52M51, either alone or in combination with paclitaxel, greatly decreased tumor volume in xenografted animals (FIG. 3B).

OMP-B40 tumors were harvested from control and OMP-52M51-treated mice with or without paclitaxel. IHC analysis was performed on tumors from OMP-B40 to detected the activated form of NOTCH1 using a rabbit polyclonal antibody that specifically recognizes NOTCH1-ICD. OMP-52M51 and OMP-52M51 plus Taxol blocks NOTCH-ICD accumulation in the nucleus (FIG. 3D). Thus, the NOTCH1-ICD IHC assay serves as key biomarker for OMP-52M51 and can be used to monitor pharmacodynamic response and NOTCH pathway activity in tumor tissues.

### Example 8

### Nuclear Localization of Activated NOTCH1 by Immunohistochemistry

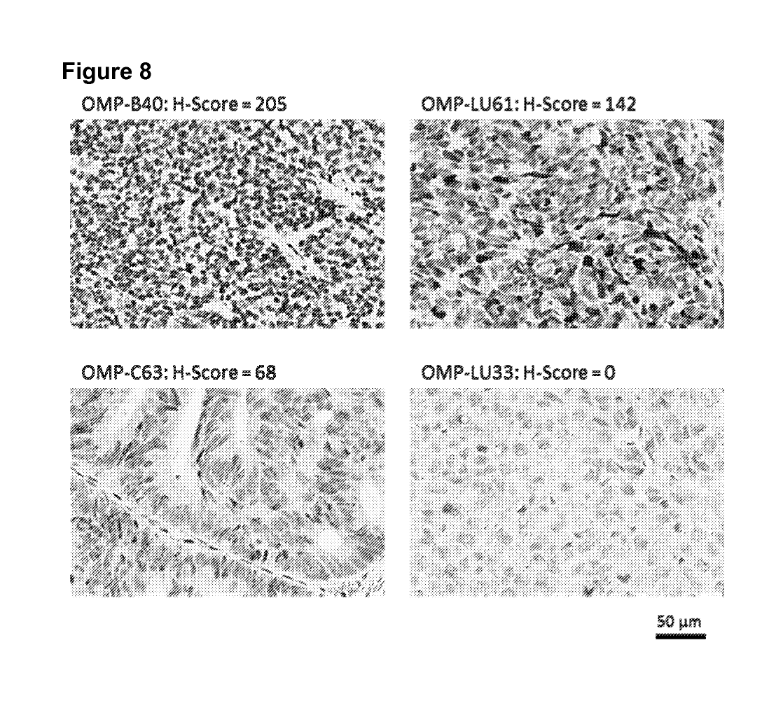

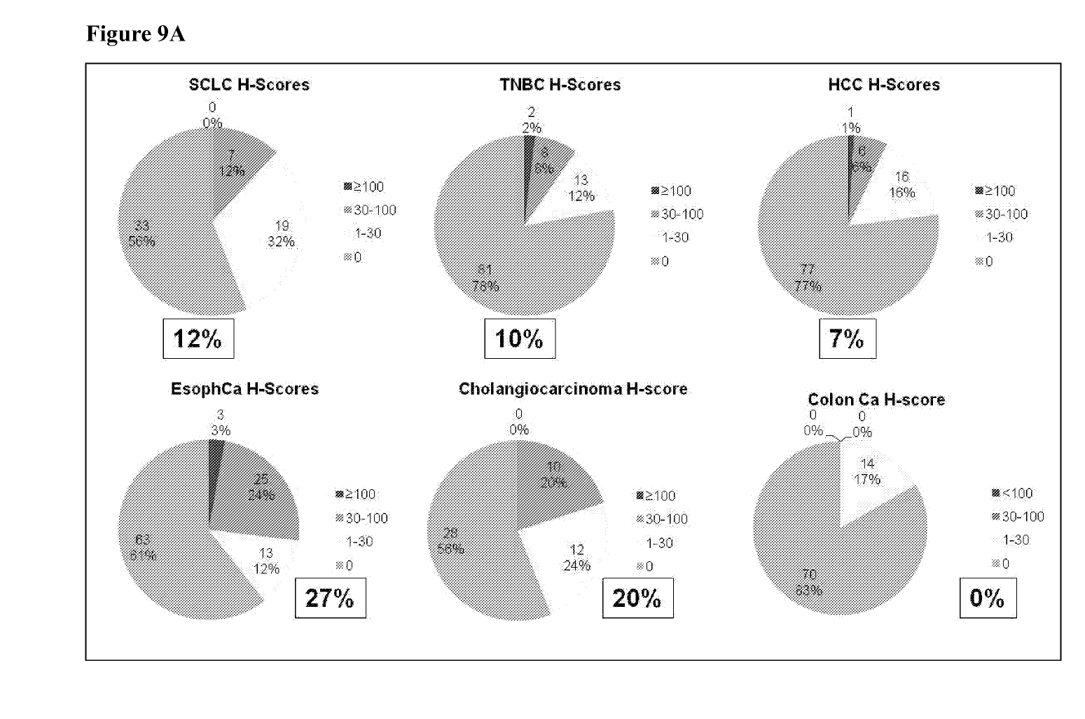

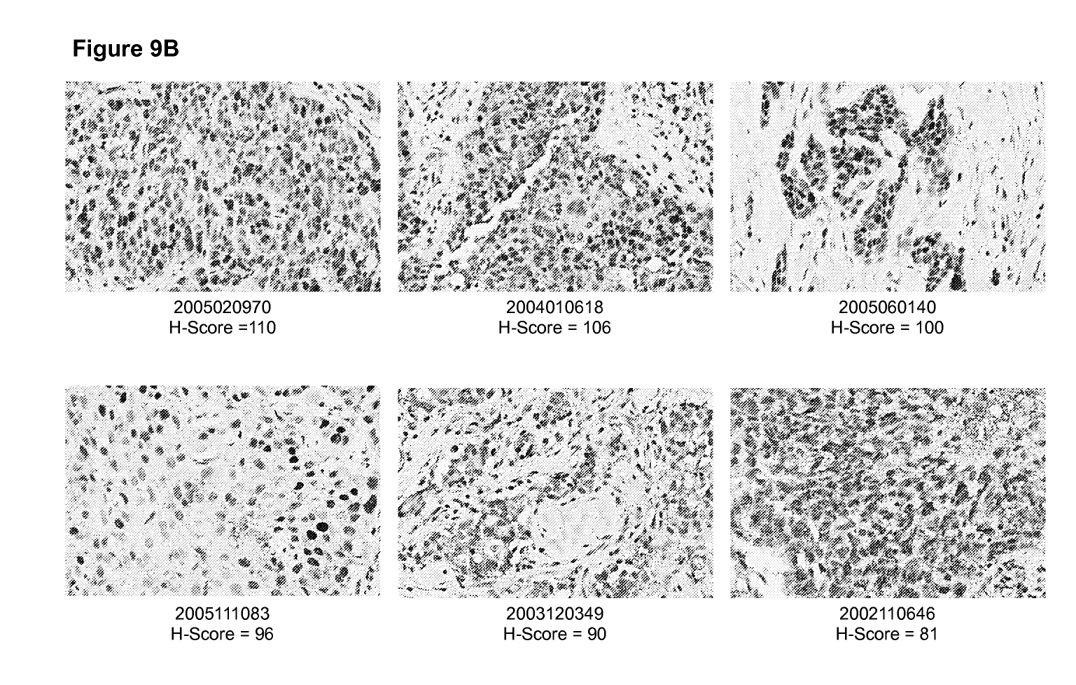

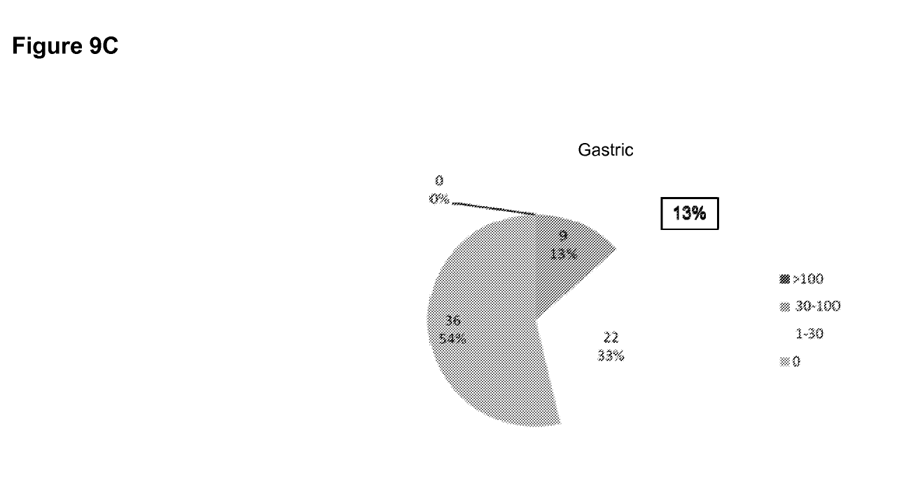

NOTCH1-ICD assay which detects the activated form of the NOTCH 1 receptor was used to screen a panel of tumors. NOTCH1-ICD was detected in OMP-B40 breast, OMP-LU61 lung, OMP-C63 colon and OMP-LU33 lung tumors (FIG. 8). OMP-LU61 and OMP-C63 do not have known NOTCH1 mutations. The IHC was quantitative and reported as an H-score. H-Score=(3×(% 3+ nuclei))+(2×(%2+ nuclei))+(1×(%1+ nuclei)). FIG. 9 shows the result of NOTCH1.ICD screening of solid tumors, including esophageal carcinoma samples. Frequence of samples with H-scores ≧30 are shown.

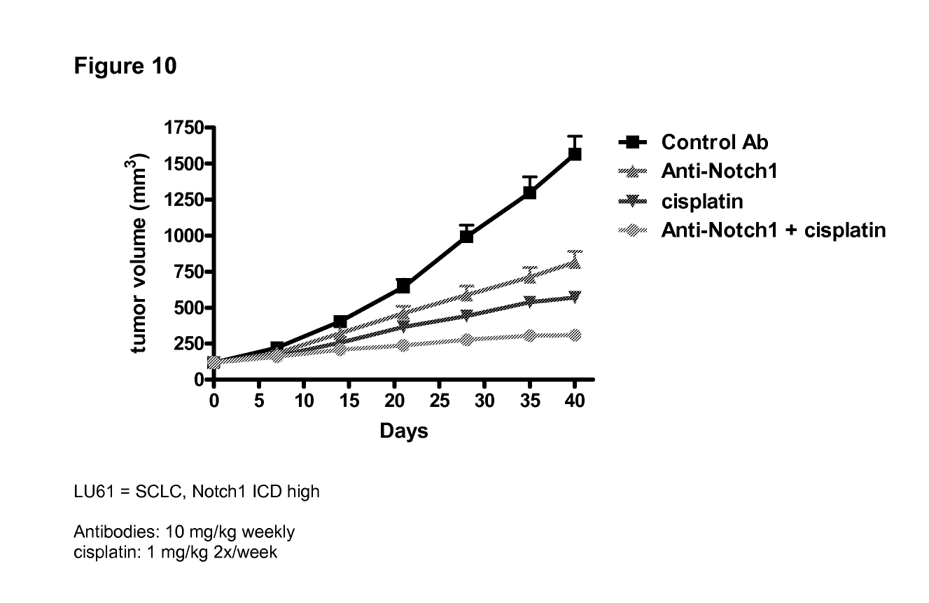

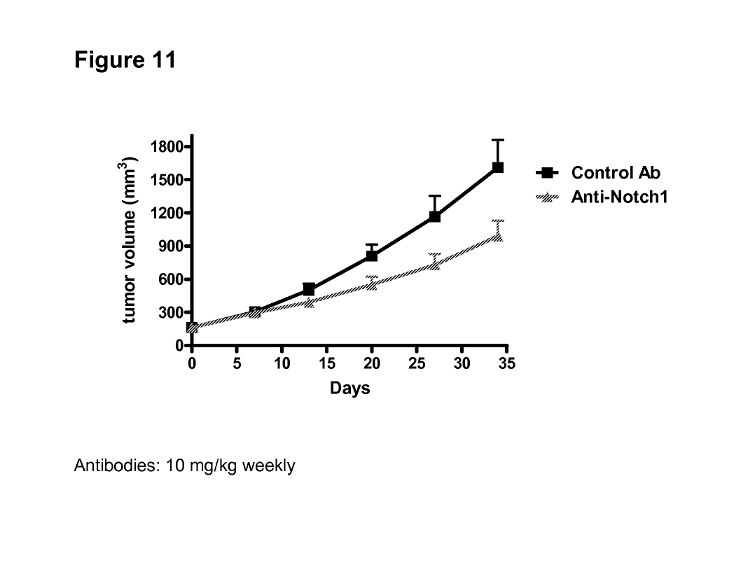

Tumors with elevated NOTCH 1-ICD including OMP-B40, OMP-LU61, and OMP-C63 showed significant sensitivity to treatment with OMP-52M51 humanized anti-NOTCH1 antibody (FIGS. 9, 10 and 11). In contrast, tumors with no NOTCH1-ICD staining (e.g. OMP-LU33) did not show significant sensitivity to treatment with OMP-52M51 anti-NOTCH1 antibody (data not shown). These data show that the NOTCH 1-ICD assay can be used as a predictive biomarker to select patients that will respond to OMP-52M51 treatment.

### Example 9

### Combination Therapy Using OMP-52M51 Humanized Antibody and Irinotecan

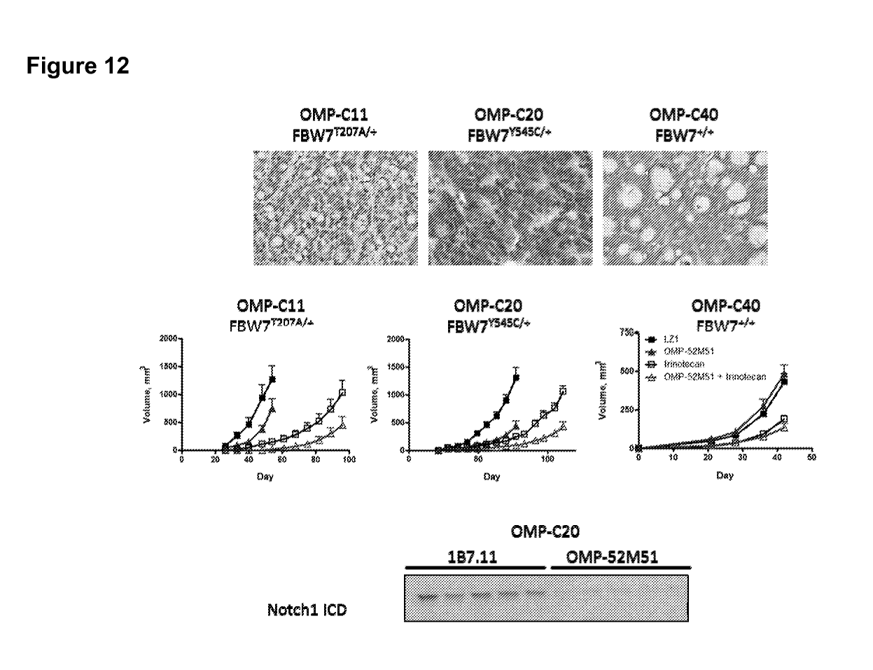

Twenty thousand OMP-C11 tumor cells were resuspended in injection media composed of PBS and 0.5× matrigel and injected subcutaneously into mice. The N1.ICD IHC H-score of OMP-C11 cells is approximately 34. The OMP-C11 cells are heterozygous mutant for FBW7. Treatment was initiated 1 day after tumor cell injection and continued for the duration of the experiment. The mice received either 15 mg/kg LZ1 control antibody or OMP-52M51 anti-NOTCH1 antibody twice weekly, either as a single therapy or in combination with 7.5 mg/kg Irinotecan administered once weekly. The antibodies and irinotecan were administered by IP injection. The results of the experiment are presented in FIG. 12. Tumor volume is shown as Mean±SEM, n=10 animals per group. OMP-52M51 alone reduced tumor growth to 53% of the tumor growth observed in the control antibody treated group (Day 54, p=0.046). The OMP-52M51 plus irinotecan combination treatment reduced tumor growth to 44% of the tumor growth observed in the group receiving the control antibody in combination with irinotecan (Day 96, p=0.044).

One hundred eighty thousand OMP-C20 tumor cells were resuspended in injection media composed of PBS and 0.5× matrigel and injected subcutaneously. The N1.ICD IHC H-score of OMP-C20 cells is approximately 58. The OMP-C20 cells are heterozygous mutant for FBW7. Treatment was initiated 1 day after tumor cell injection and continued for the duration of the experiment. The mice received either 15 mg/kg 1B7.11 control antibody or OMP-52M51 anti-NOTCH1 antibody twice weekly, either as a single therapy or in combination with 7.5 mg/kg Irinotecan administered once weekly. The antibodies and irinotecan were administered by IP injection. The results of the experiment are presented in FIG. 12. Tumor volume is shown as Mean±SEM, n=10 animals per group. OMP-52M51 alone reduced tumor growth to 34% of the tumor growth observed in the control antibody treated group (Day 70, p=<0.001). The OMP-52M51 plus irinotecan combination treatment reduced tumor growth to 41% of the tumor growth observed in the group receiving the control antibody in combination with irinotecan (Day 111, p=0.0001).

The sensitivity of OMP-C40 tumor cells to treatment with OMP-52M51 anti-NOTCH1 antibody alone or in combination with irinotecan was also determined using an in vivo xenograft model substantially as described above. The N1.ICD IHC H-score of OMP-C20 cells is approximately 23. The OMP-C40 cells carry two wild type copies of the FBW7 gene. The results of the experiment are presented in FIG. 12. There was no significant difference in tumor growth between the OMP-52M51 and control antibody treated groups.

### Example 10

### Signalling by Mutant NOTCH1 and NOTCH3 Polypeptides

Construction of NOTCH Mutant expression plasmids: NOTCH1 and NOTCH3 mutants were generated using the Agilent QuikChange II XL Site-Directed Mutagenesis Kit (Agilent; La Jolla, Calif.). PCR primers were made using the Agilent primer design site. The PCR reaction was run with 50 ng dsDNA NOTCH wild type template in pcDNA3.1 and other reaction solutions per the protocol. For adequate amplification the thermocycler was adjusted to 2.5 min/kb plasmid length at 68° C. Amplified DNA was then digested with Dpn I restriction enzyme and transformed using XL10-Gold Ultracompetent Cells. DNA was plated on LB-ampicillin plates and grown overnight. Colonies were picked and submitted to ElimBio (Hawyard, Calif.) for sequencing to confirm the presence of the mutation. Clones positive for the mutation were maxipreped to generate additional DNA. Full length sequencing was performed on the positive clones to ensure no additional mutations were present.

Luciferase Assay: PC3, HeLa, and A549 cells were placed on 10 cm dishes (1 mil cells/9 mL media) and grown overnight at 37° C./5% CO2. Cell culture medium was DMEM+high glucose, 10% FBS, 1% HEPES and 1% Pen/strep (Invitrogen; Carlsbad, Calif.). DNA transfection was prepared using OptiMEM, FuGENE 6, 2 μg hNOTCH.WT or pcDNA or NOTCH.mutants, 2 μg pGL4_{—}8 xCBS, 2 μg pcDNA3_Mammal and 0.5 μg pGL3_RL.CMV. Transfection reagents were mixed and incubated at room temperature for 15 minutes before being added to the cells. Transfected PC3 cells were incubated overnight at 37° C./5% CO2. At the same time as transfection, 96-well plates were coated with hJAG1 (31.25-500 ng) (R&D Systems; Minneapolis, Minn.) or no ligand in 30 μL PBS per well. Coated plates were stored overnight at 4° C. After 24 hr transient transfection, cells were collected and 70 μL/well were added to the 96-well coated plates before incubating overnight at 37° C./5% CO2. NOTCH activity was assessed using the Dual-Glo luciferase Assay System (Promega; Madison, Wis.). NOTCH activity was calculated taking the ratio of Firefly luciferase to Renilla luciferase.

PC3 cells were transiently transfected with DNA encoding NOTCH1 full length wild type (NOTCH1_WT) or NOTCH1_G2427fs (OMP-B40) mutant polypeptide. NOTCH mediated luciferase activation was assessed in the absence of exogenous ligand using the Dual-glo luciferase system.

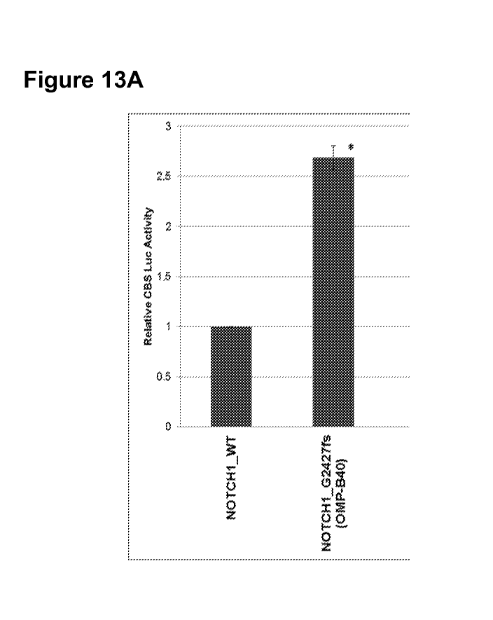

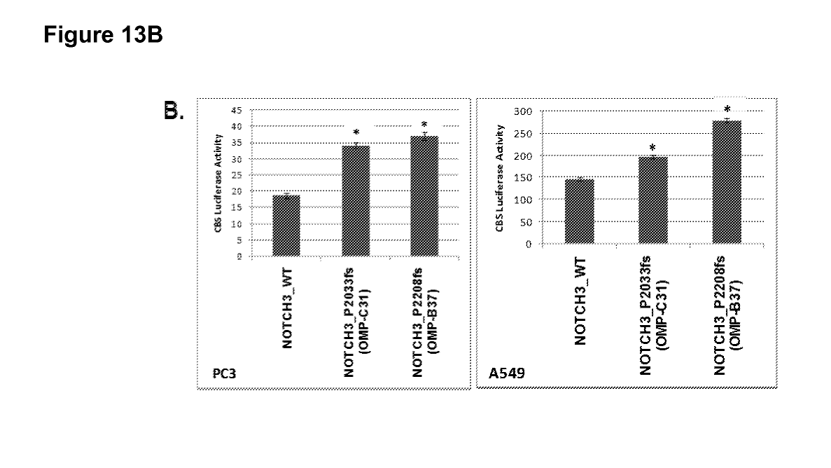

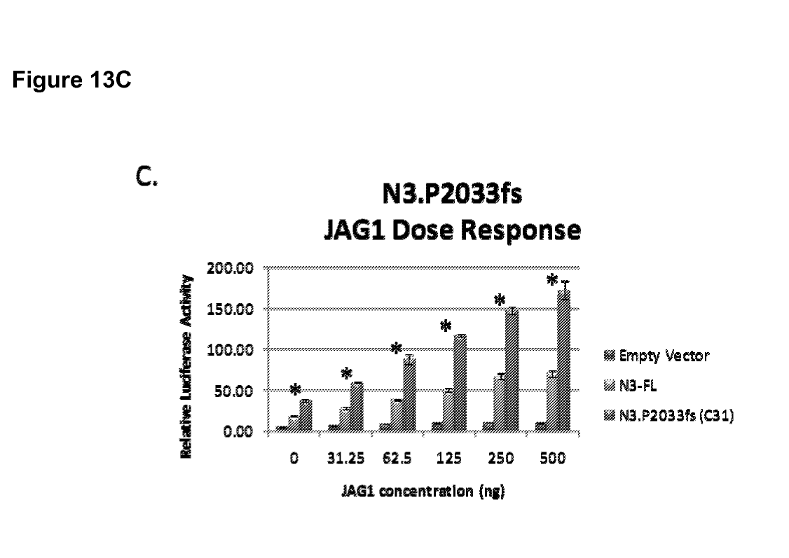

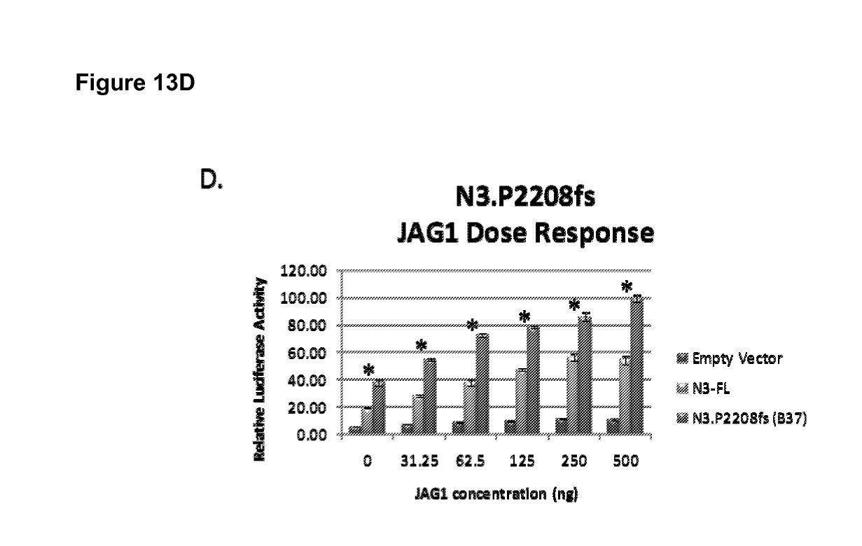

PC3 cells and A549 cells were transiently transfected with DNA encoding NOTCH3 full length wild type (NOTCH3_WT), NOTCH3_P2033fs (OMP-C31), or NOTCH3_P2208fs (OMP-B37) polypeptide. Both PC3 cells and A549 cells have very low levels of endogenous NOTCH ligand, such as DLL4 or JAG1. NOTCH mediated luciferase activation was assessed in the absence of exogenous ligand using the Dual-glo luciferase system. Results of the experiment are shown in FIG. 13B. NOTCH3_P2033fs (6096insC, OMP-C31) and NOTCH3_P2208fs (6620insC, OMP-B37) are activating mutations in both cell lines.

Dose response curves were generated for JAG1 (1-500 ng/30 μL) in PC3 cells transfected with NOTCH3_P2033fs (OMP-C31) encoding DNA (FIG. 13C). Dose response curves were generated for JAG1 (1-500 ng/30 μL) in PC3 cells transfected with NOTCH3_P2208fs (OMP-B37) encoding DNA (FIG. 13D). The JAG1 mediated signaling by the NOTCH3_P2033fs (OMP-C31) and NOTCH3_P2208fs (OMP-B37) polypeptides is significantly higher than the signaling by wild tune NOTCH3.

### Example 10

### Signalling by Mutant NOTCH1 and NOTCH3 Polypeptides

Construction of NOTCH Mutant expression plasmids: NOTCH1 and NOTCH3 mutants were generated using the Agilent QuikChange II XL Site-Directed Mutagenesis Kit (Agilent; La Jolla, Calif.). PCR primers were made using the Agilent primer design site. The PCR reaction was run with 50 ng dsDNA NOTCH wild type template in pcDNA3.1 and other reaction solutions per the protocol. For adequate amplification the thermocycler was adjusted to 2.5 min/kb plasmid length at 68° C. Amplified DNA was then digested with Dpn I restriction enzyme and transformed using XL10-Gold Ultracompetent Cells. DNA was plated on LB-ampicillin plates and grown overnight. Colonies were picked and submitted to ElimBio (Hawyard, Calif.) for sequencing to confirm the presence of the mutation. Clones positive for the mutation were maxipreped to generate additional DNA. Full length sequencing was performed on the positive clones to ensure no additional mutations were present.

### Example 11

### Activity of NOTCH1 Antagonists in OMP-B40 Tumors

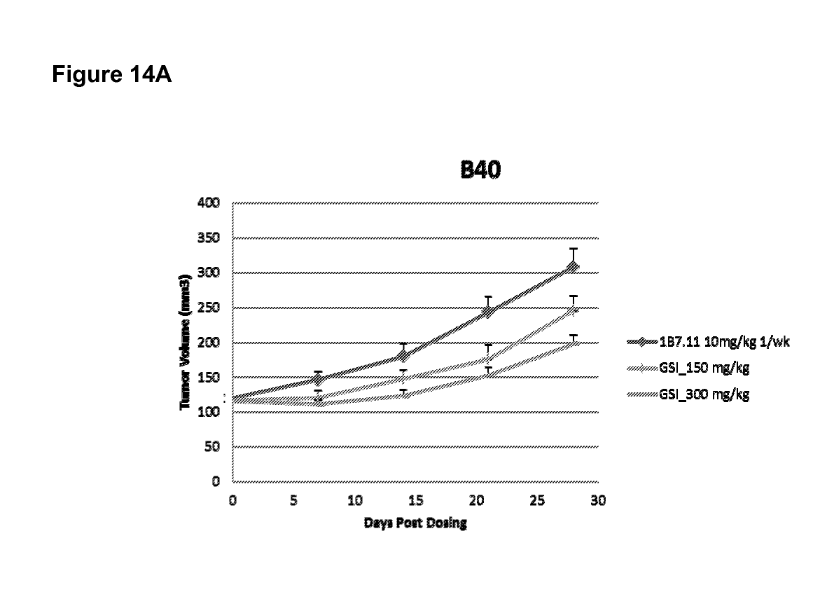

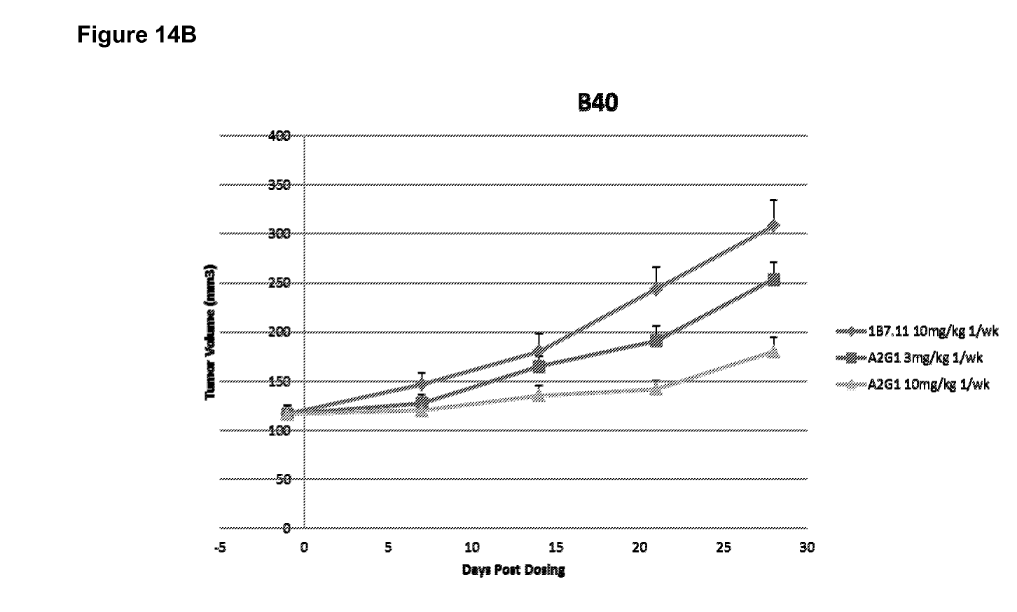

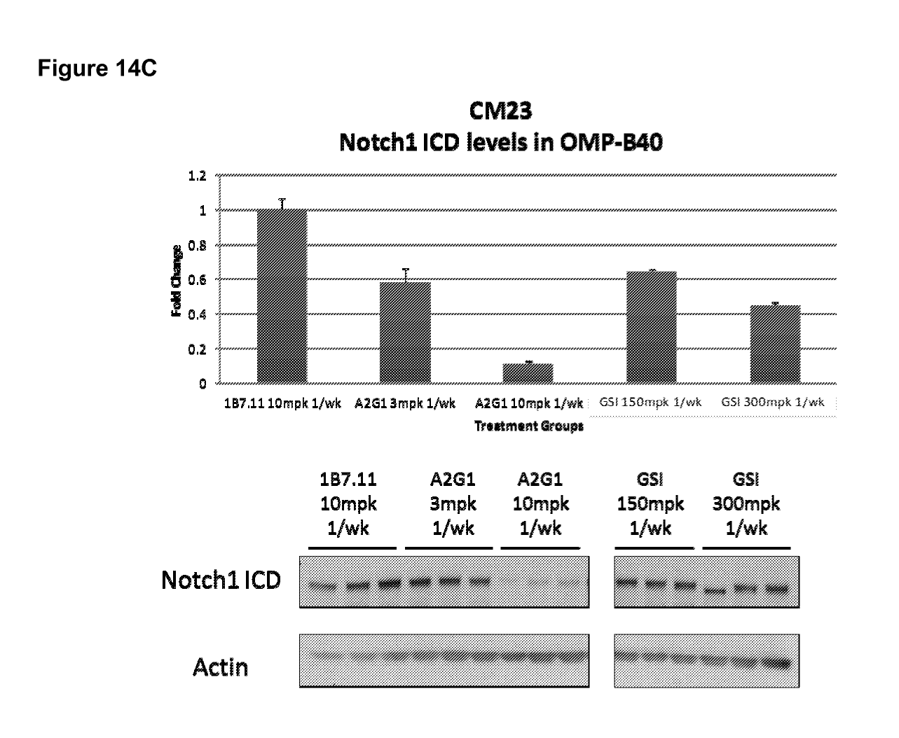

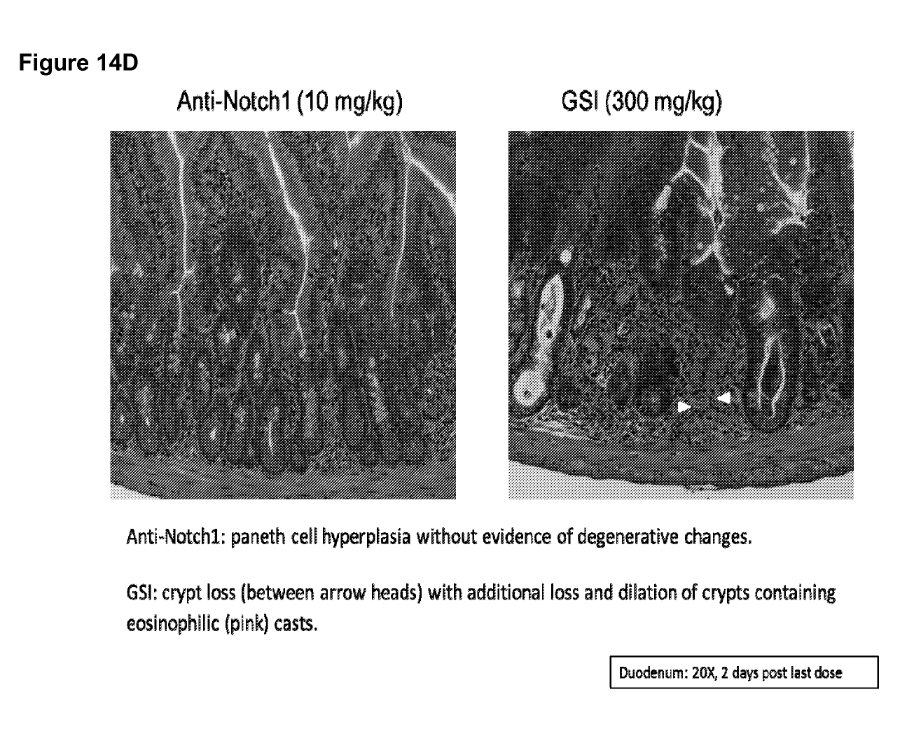

To test the effect of an anti-NOTCH1 antibody in comparison with a gamma-secretase inhibitor we utilized the OMP-B40 breast tumor model which harbors an activating mutation in NOTCH1. 75,000 OMP-B40 breast tumor cells were injected in Nod-Scid mice. Tumors were allowed to grow for 50 days until they had reached an average volume of 120 mm³ Tumor bearing mice (n=10 group) were treated with either control antibody (1B7.11, 10 mg/kg) weekly or every other week); anti-NOTCH1 (A2G1, an anti-NOTCH1 antibody that recognizes both murine Notch1 and human NOTCH1), 3 mg/kg or 10 mg/kg, weekly or every other week; or a gamma secretase inhibitor (GSI) at either 150 mg/kg or 300 mg/kg, 5 times per week. Antibodies were dosed by IP injection and the GSI compound was dosed by oral gavage. Tumor volumes were measured on the indicated days. The data is plotted as the mean+SEM. Both the GSI and A2G1 anti-NOTCH1 antibody inhibited tumor growth as shown in FIGS. 14A and B, respectively. The extent of tumor growth inhibition by A2G1 at 10 mg/kg weekly and GSI at 300 mg/kg was similar.

Tumor lysates were analyzed for the presence of activated NOTCH1 by western blot with an antibody that selectively recognizes the cleaved form of the NOTCH1 ICD (FIG. 14C). The A2G1 anti-NOTCH1 antibody was a more effective inhibitor than the gamma-secretase inhibitor of NOTCH1 activation.

The effect on gastrointestinal toxicity was also examined by histological analyses (FIG. 14D). The A2G1 anti-NOTCH1 antibody at 10 mg/kg weekly resulted in less severe gastrointestinal toxicity than GSI at 300 mg/kg (qdX5). Thus, selective NOTCH1 inhibition by a monoclonal antibody is better tolerated and is more effective than a GSI at inhibiting NOTCH1 signaling in tumors bearing activating NOTCH1 mutations.

### Example 12

### Activity of 59R5 Anti-NOTCH2/3 Antibody in OMP-C31 Colon Tumor

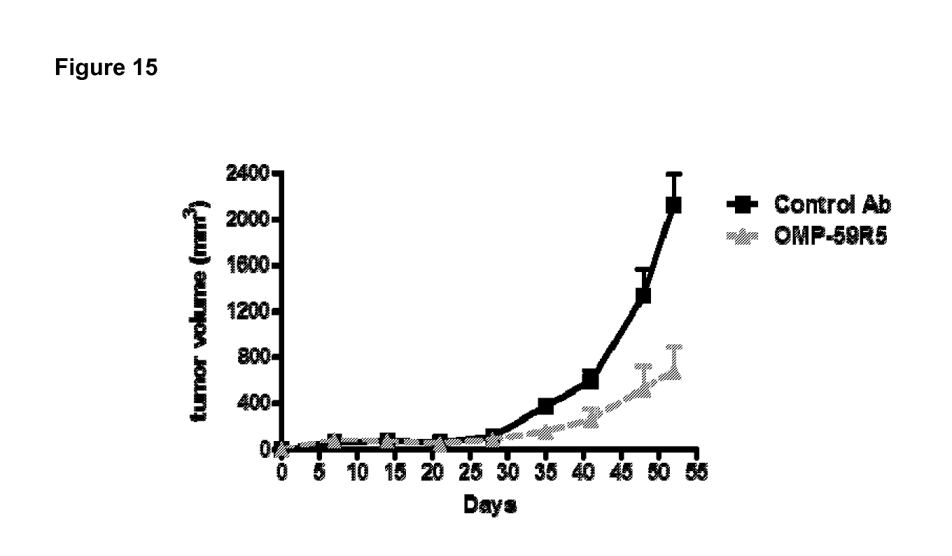

In order to test the effect of the 59R5 anti-NOTCH2/3 antibody on a tumor which harbors an activating mutation in NOTCH3, the OMP-C31 colon tumor model was utilized. 5,000 OMP-C31 colon tumor cells were injected sub-cutaneously in Nod-Scid mice. Dosing was initiated two days after tumor cell injection. Tumor bearing mice (n=10 group) were treated with either control antibody (1B7.11) or 59R5 anti-NOTCH2/3 antibody. Antibodies were dosed by IP injection at 10 mg/kg, weekly, for the duration of the experiment. Tumor volumes were measured on the indicated days. The data is plotted as the mean+SEM (FIG. 15). 59R5 anti-NOTCH2/3 antibody inhibited OMP-C31 tumor growth relative to the control antibody treated group (p=0.0005).

### Example 13

### 52M51 Anti-NOTCH1 Antibody Inhibits Ligand Mediated Signaling by the G2427fs and R2328W Mutant NOTCH1 Polypeptide

In certain embodiments, the ability of the 52M51 NOTCH1 receptor antibody to block ligand-mediated signaling by the G2427fs and R2328W (Westhoff et al., Proc Natl Acad Sci 2009 Dec. 29; 106(52): 22293-22298) mutant NOTCH1 polypeptide was determined PC3 cells were co-transfected with (a) a vector expressing G2427fs, R2328W, or wild type NOTCH1, (b) the pGL4_{—}8 xCBS vector comprising a NOTCH responsive promoter upstream of a firefly luciferase reporter gene, (c) the pcDNA3_Mammal vector expressing MAML, a transcription co-factor for NOTCH, and (d) the pGL3_RL.CMV vector expressing Renilla luciferase. Control cells were transfected with an empty vector in place of (a). DNA transfection was prepared using OptiMEM, FuGENE 6. Transfection reagents were mixed and incubated at room temperature for 15 minutes before being added to the cells. Transfected PC3 cells were incubated overnight at 37° C./5% CO₂. At the time of adding the transfection reagents to the cells, 96-well plates were coated with hDLL4 (12.5 ng) or hJAG1 (125 ng) (R&D Systems; Minneapolis, Minn.) or no ligand in 30 μL PBS per well. Coated plates were stored overnight at 4° C. After 24 hour transient transfection, cells were collected and 70 μL/well were added to the 96-well coated plates before incubating overnight at 37° C./5% CO₂. At least 1 hour prior to addition of the transfected cells, 10 uL/well (1.6-1000 ng/mL) 52M51 anti-NOTCH1 antibody was added into the 96 well plates. NOTCH activity was assessed using the Dual-Glo luciferase Assay System (Promega; Madison, Wis.). NOTCH activity was calculated by determining the ratio of Firefly luciferase to Renilla luciferase activities.

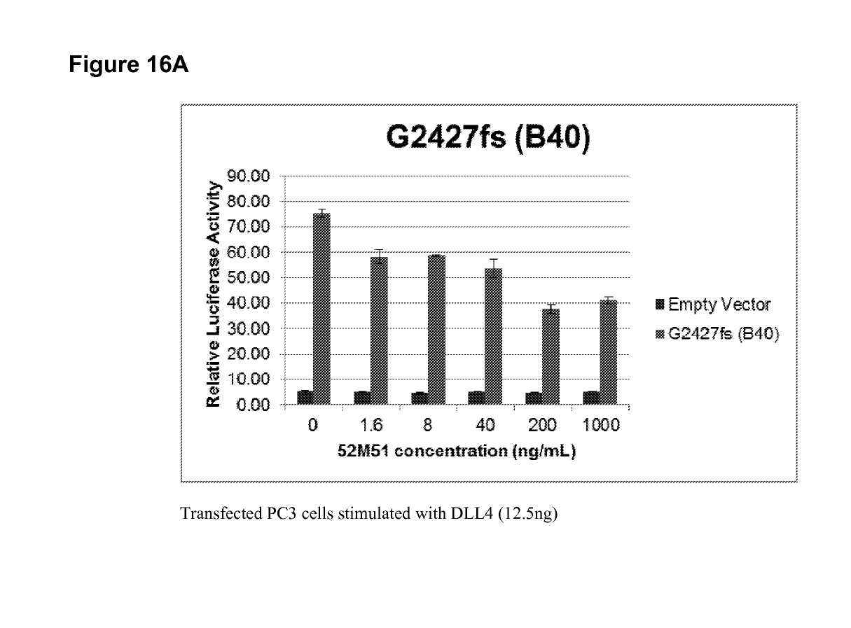

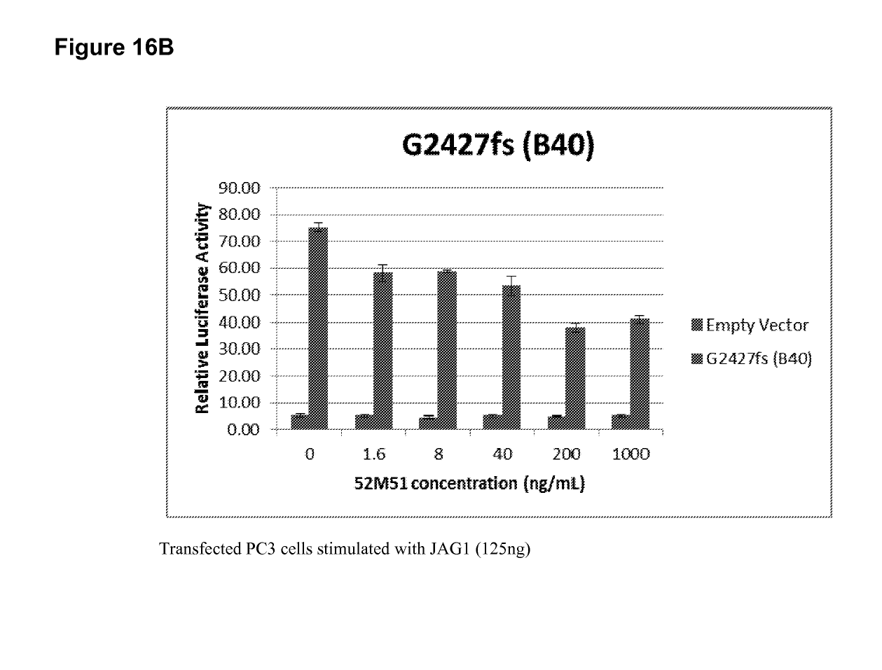

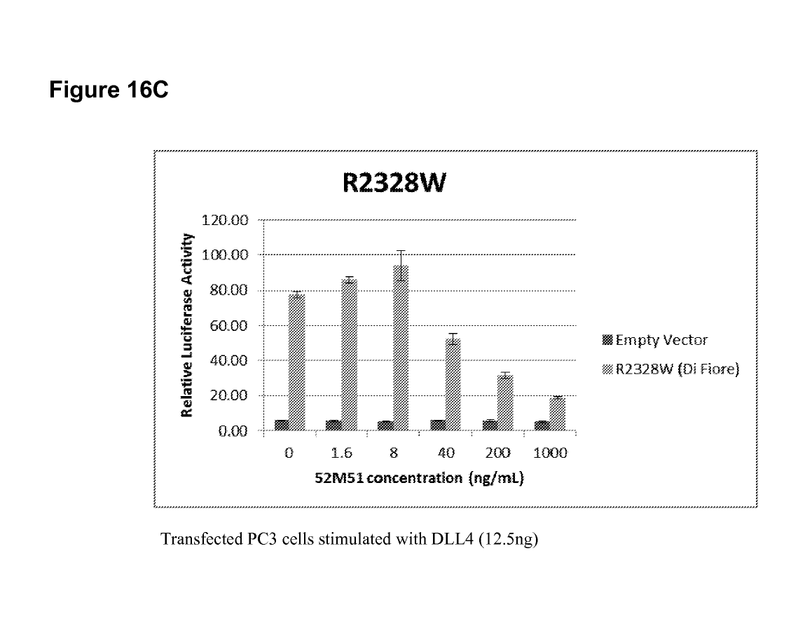

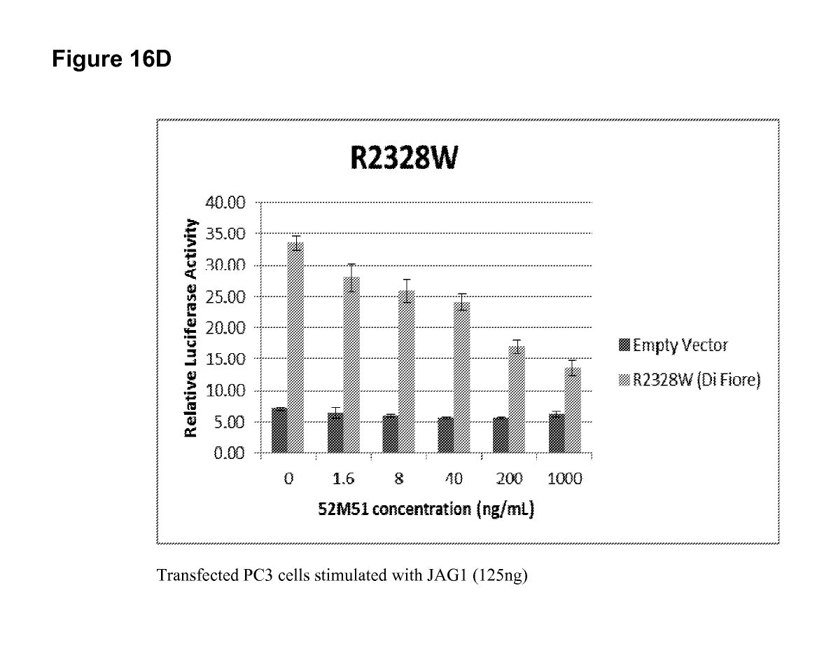

FIGS. 16A and B show the Firefly luciferase to Renilla luciferase activity ratio observed in G2427fs (OMP-B40) NOTCH1 mutant polypeptide expressing PC3 cells after stimulation with DLL4 and JAG1, respectively. FIGS. 16C and D show the Firefly luciferase to Renilla luciferase activity ratio observed in R2328W NOTCH1 mutant polypeptide expressing PC3 cells after stimulation with DLL4 and JAG1, respectively. PC3 cells expressing G2427fs or R2328W NOTCH1 had significantly higher Firefly luciferase to Renilla luciferase activity ratio than cells not expressing a recombinant NOTCH1. Further, the 52M51 anti-NOTCH1 antibody reduced the G2427fs and R2328W NOTCH1 polypeptide mediated increase in Firefly luciferase to Renilla luciferase activity ratio in a dose dependent manner.

### Example 14

### NOTCH3 ICD IHC Analysis of Treated OMP-B37 Breast Tumors

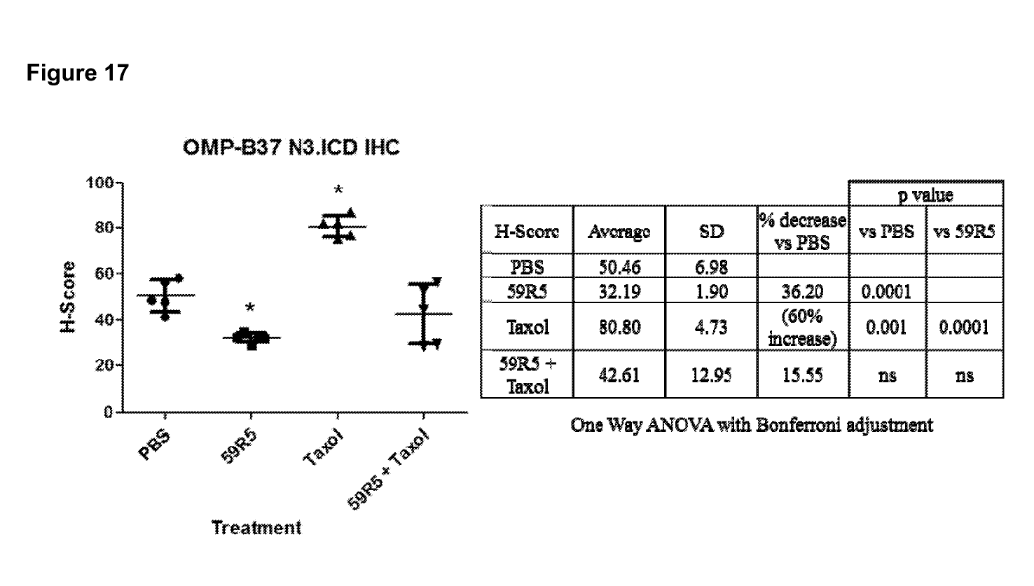

NOTCH3.ICD accumulation in OMP-B37 tumor cells comprising a NOTCH3 activating mutation following treatment with a NOTCH2/3 antagonist and/or chemotherapy was examined by IHC. Tumor samples were isolated from the OMP-B37 xenograft mice that were treated with OMP-59R5 anti-NOTCH2/3 antibody, taxol or a combination of OMP-59R5 and taxol as described in the second paragraph of Example 3 above. A NOTCH3 ICD (“N3.ICD”) rabbit monoclonal antibody was developed that binds to the cleavage site of NOTCH3 and detects activated NOTCH3 within the nucleus. NOTCH3 ICD IHC was performed on the tumor samples using the N3.ICD antibody and standard IHC protocols with antigen retrieval in Target Retrieval Solution pH 9 (DAKO), antibody incubation overnight at 4° C. and detection with peroxidase-based EnVision™+ polymer (DAKO) and DAB+ (DAKO). Statistical significance was determined by One Way ANOVA with Bonferroni adjustment. Results obtained are shown in FIG. 17. OMP-59R5 anti-NOTCH2/3 antibody treatment significantly inhibited NOTCH3.ICD accumulation in OMP-B37 breast tumor cells relative to the NOTCH3.ICD accumulation in PBS treated cells. The OMP-59R5 anti-NOTCH2/3 plus taxol combination treatment significantly inhibited NOTCH3.ICD accumulation in OMP-B37 cells relative to the accumulation detected in taxol only treated cells.

Human NOTCH1 gene (wild-type)
SEQ ID NO: 1
atgccgccgctcctggcgcccctgctctgcctggcgctgctgcccgcgctcgccgcacgaggcccgcg
atgctcccagcccggtgagacctgcctgaatggcgggaagtgtgaagcggccaatggcacggaggcct
gcgtctgtggcggggccttcgtgggcccgcgatgccaggaccccaacccgtgcctcagcaccccctgc
aagaacgccgggacatgccacgtggtggaccgcagaggcgtggcagactatgcctgcagctgtgccct
gggcttctctgggcccctctgcctgacacccctggacaatgcctgcctcaccaacccctgccgcaacg
ggggcacctgcgacctgctcacgctgacggagtacaagtgccgctgcccgcccggctggtcagggaaa
tcgtgccagcaggctgacccgtgcgcctccaacccctgcgccaacggtggccagtgcctgcccttcga
ggcctcctacatctgccactgcccacccagcttccatggccccacctgccggcaggatgtcaacgagt
gtggccagaagcccgggctttgccgccacggaggcacctgccacaacgaggtcggctcctaccgctgc
gtctgccgcgccacccacactggccccaactgcgagcggccctacgtgccctgcagcccctcgccctg
ccagaacgggggcacctgccgccccacgggcgacgtcacccacgagtgtgcctgcctgccaggcttca
ccggccagaactgtgaggaaaatatcgacgattgtccaggaaacaactgcaagaacgggggtgcctgt
gtggacggcgtgaacacctacaactgccgctgcccgccagagtggacaggtcagtactgtaccgagga
tgtggacgagtgccagctgatgccaaatgcctgccagaacggcgggacctgccacaacacccacggtg
gctacaactgcgtgtgtgtcaacggctggactggtgaggactgcagcgagaacattgatgactgtgcc
agcgccgcctgcttccacggcgccacctgccatgaccgtgtggcctccttctactgcgagtgtcccca
tggccgcacaggtctgctgtgccacctcaacgacgcatgcatcagcaacccctgtaacgagggctcca
actgcgacaccaaccctgtcaatggcaaggccatctgcacctgcccctcggggtacacgggcccggcc
tgcagccaggacgtggatgagtgctcgctgggtgccaacccctgcgagcatgcgggcaagtgcatcaa
cacgctgggctccttcgagtgccagtgtctgcagggctacacgggcccccgatgcgagatcgacgtca
acgagtgcgtctcgaacccgtgccagaacgacgccacctgcctggaccagattggggagttccagtgc
atctgcatgcccggctacgagggtgtgcactgcgaggtcaacacagacgagtgtgccagcagcccctg
cctgcacaatggccgctgcctggacaagatcaatgagttccagtgcgagtgccccacgggcttcactg
ggcatctgtgccagtacgatgtggacgagtgtgccagcaccccctgcaagaatggtgccaagtgcctg
gacggacccaacacttacacctgtgtgtgcacggaagggtacacggggacgcactgcgaggtggacat
cgatgagtgcgaccccgacccctgccactacggctcctgcaaggacggcgtcgccaccttcacctgcc
tctgccgcccaggctacacgggccaccactgcgagaccaacatcaacgagtgctccagccagccctgc
cgccacgggggcacctgccaggaccgcgacaacgcctacctctgcttctgcctgaaggggaccacagg
acccaactgcgagatcaacctggatgactgtgccagcagcccctgcgactcgggcacctgtctggaca
agatcgatggctacgagtgtgcctgtgagccgggctacacagggagcatgtgtaacatcaacatcgat
gagtgtgcgggcaacccctgccacaacgggggcacctgcgaggacggcatcaatggcttcacctgccg
ctgccccgagggctaccacgaccccacctgcctgtctgaggtcaatgagtgcaacagcaacccctgcg
tccacggggcctgccgggacagcctcaacgggtacaagtgcgactgtgaccctgggtggagtgggacc
aactgtgacatcaacaacaatgagtgtgaatccaacccttgtgtcaacggcggcacctgcaaagacat
gaccagtggctacgtgtgcacctgccgggagggcttcagcggtcccaactgccagaccaacatcaacg
agtgtgcgtccaacccatgtctgaaccagggcacgtgtattgacgacgttgccgggtacaagtgcaac
tgcctgctgccctacacaggtgccacgtgtgaggtggtgctggccccgtgtgcccccagcccctgcag
aaacggcggggagtgcaggcaatccgaggactatgagagcttctcctgtgtctgccccacgggctggc
aagggcagacctgtgaggtcgacatcaacgagtgcgttctgagcccgtgccggcacggcgcatcctgc
cagaacacccacggcggctaccgctgccactgccaggccggctacagtgggcgcaactgcgagaccga
catcgacgactgccggcccaacccgtgtcacaacgggggctcctgcacagacggcatcaacacggcct
tctgcgactgcctgcccggcttccggggcactttctgtgaggaggacatcaacgagtgtgccagtgac
ccctgccgcaacggggccaactgcacggactgcgtggacagctacacgtgcacctgccccgcaggctt
cagcgggatccactgtgagaacaacacgcctgactgcacagagagctcctgcttcaacggtggcacct
gcgtggacggcatcaactcgttcacctgcctgtgtccacccggcttcacgggcagctactgccagcac
gatgtcaatgagtgcgactcacagccctgcctgcatggcggcacctgtcaggacggctgcggctccta
caggtgcacctgcccccagggctacactggccccaactgccagaaccttgtgcactggtgtgactcct
cgccctgcaagaacggcggcaaatgctggcagacccacacccagtaccgctgcgagtgccccagcggc
tggaccggcctttactgcgacgtgcccagcgtgtcctgtgaggtggctgcgcagcgacaaggtgttga
cgttgcccgcctgtgccagcatggagggctctgtgtggacgcgggcaacacgcaccactgccgctgcc
aggcgggctacacaggcagctactgtgaggacctggtggacgagtgctcacccagcccctgccagaac
ggggccacctgcacggactacctgggcggctactcctgcaagtgcgtggccggctaccacggggtgaa
ctgctctgaggagatcgacgagtgcctctcccacccctgccagaacgggggcacctgcctcgacctcc
ccaacacctacaagtgctcctgcccacggggcactcagggtgtgcactgtgagatcaacgtggacgac
tgcaatccccccgttgaccccgtgtcccggagccccaagtgctttaacaacggcacctgcgtggacca
ggtgggcggctacagctgcacctgcccgccgggcttcgtgggtgagcgctgtgagggggatgtcaacg
agtgcctgtccaatccctgcgacgcccgtggcacccagaactgcgtgcagcgcgtcaatgacttccac
tgcgagtgccgtgctggtcacaccgggcgccgctgcgagtccgtcatcaatggctgcaaaggcaagcc
ctgcaagaatgggggcacctgcgccgtggcctccaacaccgcccgcgggttcatctgcaagtgccctg
cgggcttcgagggcgccacgtgtgagaatgacgctcgtacctgcggcagcctgcgctgcctcaacggc
ggcacatgcatctccggcccgcgcagccccacctgcctgtgcctgggccccttcacgggccccgaatg
ccagttcccggccagcagcccctgcctgggcggcaacccctgctacaaccaggggacctgtgagccca
catccgagagccccttctaccgttgcctgtgccccgccaaattcaacgggctcttgtgccacatcctg
gactacagcttcgggggtggggccgggcgcgacatccccccgccgctgatcgaggaggcgtgcgagct
gcccgagtgccaggaggacgcgggcaacaaggtctgcagcctgcagtgcaacaaccacgcgtgcggct
gggacggcggtgactgctccctcaacttcaatgacccctggaagaactgcacgcagtctctgcagtgc
tggaagtacttcagtgacggccactgtgacagccagtgcaactcagccggctgcctcttcgacggctt
tgactgccagcgtgcggaaggccagtgcaaccccctgtacgaccagtactgcaaggaccacttcagcg
acgggcactgcgaccagggctgcaacagcgcggagtgcgagtgggacgggctggactgtgcggagcat
gtacccgagaggctggcggccggcacgctggtggtggtggtgctgatgccgccggagcagctgcgcaa
cagctccttccacttcctgcgggagctcagccgcgtgctgcacaccaacgtggtcttcaagcgtgacg
cacacggccagcagatgatettcccctactacggccgcgaggaggagctgcgcaagcaccccatcaag
cgtgccgccgagggctgggccgcacctgacgccctgctgggccaggtgaaggcctcgctgctccctgg
tggcagcgagggtgggcggcggcggagggagctggaccccatggacgtccgcggctccatcgtctacc
tggagattgacaaccggcagtgtgtgcaggcctcctcgcagtgcttccagagtgccaccgacgtggcc
gcattcctgggagcgctcgcctcgctgggcagcctcaacatcccctacaagatcgaggccgtgcagag
tgagaccgtggagccgcccccgccggcgcagctgcacttcatgtacgtggcggcggccgcctttgtgc
ttctgttcttcgtgggctgcggggtgctgctgtcccgcaagcgccggcggcagcatggccagctctgg
ttccctgagggcttcaaagtgtctgaggccagcaagaagaagcggcgggagcccctcggcgaggactc
cgtgggcctcaagcccctgaagaacgcttcagacggtgccctcatggacgacaaccagaatgagtggg
gggacgaggacctggagaccaagaagttccggttcgaggagcccgtggttctgcctgacctggacgac
cagacagaccaccggcagtggactcagcagcacctggatgccgctgacctgcgcatgtctgccatggc
ccccacaccgccccagggtgaggttgacgccgactgcatggacgtcaatgtccgcgggcctgatggct
tcaccccgctcatgatcgcctcctgcagcgggggcggcctggagacgggcaacagcgaggaagaggag
gacgcgccggccgtcatctccgacttcatctaccagggcgccagcctgcacaaccagacagaccgcac
gggcgagaccgccttgcacctggccgcccgctactcacgctctgatgccgccaagcgcctgctggagg
ccagcgcagatgccaacatccaggacaacatgggccgcaccccgctgcatgcggctgtgtctgccgac
gcacaaggtgtcttccagatcctgatccggaaccgagccacagacctggatgcccgcatgcatgatgg
cacgacgccactgatcctggctgcccgcctggccgtggagggcatgctggaggacctcatcaactcac
acgccgacgtcaacgccgtagatgacctgggcaagtccgccctgcactgggccgccgccgtgaacaat
gtggatgccgcagttgtgctcctgaagaacggggctaacaaagatatgcagaacaacagggaggagac
acccctgtttctggccgcccgggagggcagctacgagaccgccaaggtgctgctggaccactttgcca
accgggacatcacggatcatatggaccgcctgccgcgcgacatcgcacaggagcgcatgcatcacgac
atcgtgaggctgctggacgagtacaacctggtgcgcagcccgcagctgcacggagccccgctgggggg
cacgcccaccctgtcgcccccgctctgctcgcccaacggctacctgggcagcctcaagcccggcgtgc
agggcaagaaggtccgcaagcccagcagcaaaggcctggcctgtggaagcaaggaggccaaggacctc
aaggcacggaggaagaagtcccaggacggcaagggctgcctgctggacagctccggcatgctctcgcc
cgtggactccctggagtcaccccatggctacctgtcagacgtggcctcgccgccactgctgccctccc
cgttccagcagtctccgtccgtgcccctcaaccacctgcctgggatgcccgacacccacctgggcatc
gggcacctgaacgtggcggccaagcccgagatggcggcgctgggtgggggcggccggctggcctttga
gactggcccacctcgtctctcccacctgcctgtggcctctggcaccagcaccgtcctgggctccagca
gcggaggggccctgaatttcactgtgggcgggtccaccagtttgaatggtcaatgcgagtggctgtcc
cggctgcagagcggcatggtgccgaaccaatacaaccctctgcgggggagtgtggcaccaggccccct
gagcacacaggccccctccctgcagcatggcatggtaggcccgctgcacagtagccttgctgccagcg
ccctgtcccagatgatgagctaccagggcctgcccagcacccggctggccacccagcctcacctggtg
cagacccagcaggtgcagccacaaaacttacagatgcagcagcagaacctgcagccagcaaacatcca
gcagcagcaaagcctgcagccgccaccaccaccaccacagccgcaccttggcgtgagctcagcagcca
gcggccacctgggccggagcttcctgagtggagagccgagccaggcagacgtgcagccactgggcccc
agcagcctggcggtgcacactattctgccccaggagagccccgccctgcccacgtcgctgccatcctc
gctggtcccacccgtgaccgcagcccagttcctgacgcccccctcgcagcacagctactcctcgcctg
tggacaacacccccagccaccagctacaggtgcctgagcaccccttcctcaccccgtcccctgagtcc
cctgaccagtggtccagctcgtccccgcattccaacgtctccgactggtccgagggcgtctccagccc
tcccaccagcatgcagtcccagatcgcccgcattccggaggccttcaagtaaacggcgcgccccacga
gaccccggcttcctttcccaagccttcgggcgtctgtgtgcgctctgtggatgccagggccgaccaga
ggagcctttttaaaacacatgtttttatacaaaataagaacgaggattttaattttttttagtattta
tttatgtacttttattttacacagaaacactgcctttttatttatatgtactgttttatctggcccca
ggtagaaacttttatctattctgagaaaacaagcaagttctgagagccagggttttcctacgtaggat
gaaaagattcttctgtgtttataaaatataaacaaagattcatgatttataaatgccatttatttatt
gattccttttttcaaaatccaaaaagaaatgatgttggagaagggaagttgaacgagcatagtccaaa
aagctcctggggcgtccaggccgcgccctttccccgacgcccacccaaccccaagccagcccggccgc
tccaccagcatcacctgcctgttaggagaagctgcatccagaggcaaacggaggcaaagctggctcac
cttccgcacgcggattaatttgcatctgaaataggaaacaagtgaaagcatatgggttagatgttgcc
atgtgttttagatggtttcttgcaagcatgcttgtgaaaatgtgttctcggagtgtgtatgccaagag
tgcacccatggtaccaatcatgaatctttgtttcaggttcagtattatgtagttgttcgttggttata
caagttcttggtccctccagaaccaccccggccccctgcccgttcttgaaatgtaggcatcatgcatg
tcaaacatgagatgtgtggactgtggcacttgcctgggtcacacacggaggcatcctacccttttctg
gggaaagacactgcctgggctgaccccggtggcggccccagcacctcagcctgcacagtgtcccccag
gttccgaagaagatgctccagcaacacagcctgggccccagctcgcgggacccgaccccccgtgggct
cccgtgttttgtaggagacttgccagagccgggcacattgagctgtgcaacgccgtgggctgcgtcct
ttggtcctgtccccgcagccctggcagggggcatgcggtcgggcaggggctggagggaggcgggggct
gcccttgggccacccctcctagtttgggaggagcagatttttgcaataccaagtatagcctatggcag
aaaaaatgtctgtaaatatgtttttaaaggtggattttgtttaaaaaatcttaatgaatgagtctgtt
gtgtgtcatgccagtgagggacgtcagacttggctcagctcggggagccttagccgcccatgcactgg
ggacgctccgctgccgtgccgcctgcactcctcagggcagcctcccccggctctacgggggccgcgtg
gtgccatccccagggggcatgaccagatgcgtcccaagatgttgatttttactgtgttttataaaata
gagtgtagtttacagaaaaagactttaaaagtgatctacatgaggaactgtagatgatgtattttttt
catcttttttgttaactgatttgcaataaaaatgatactgatggtgaaaaaaaaaaaaaaa
Human NOTCH2 gene (wild-type)
SEQ ID NO: 2
gcgaccgagaagatgcccgccctgcgccccgctctgctgtgggcgctgctggcgctctggctgtgctg
cgcgacccccgcgcatgcattgcagtgtcgagatggctatgaaccctgtgtaaatgaaggaatgtgtg
ttacctaccacaatggcacaggatactgcaaatgtccagaaggcttcttgggggaatattgtcaacat
cgagacccctgtgagaagaaccgctgccagaatggtgggacttgtgtggcccaggccatgctggggaa
agccacgtgccgatgtgcctcagggtttacaggagaggactgccagtactcgacatctcatccatgct
ttgtgtctcgaccctgcctgaatggcggcacatgccatatgctcagccgggatacctatgagtgcacc
tgtcaagtcgggtttacaggtaaggagtgccaatggaccgatgcctgcctgtctcatccctgtgcaaa
tggaagtacctgtaccactgtggccaaccagttctcctgcaaatgcctcacaggcttcacagggcaga
aatgtgagactgatgtcaatgagtgtgacattccaggacactgccagcatggtggcacctgcctcaac
ctgcctggttcctaccagtgccagtgccttcagggcttcacaggccagtactgtgacagcctgtatgt
gccctgtgcaccctcgccttgtgtcaatggaggcacctgtcggcagactggtgacttcacttttgagt
gcaactgccttccaggttttgaagggagcacctgtgagaggaatattgatgactgccctaaccacagg
tgtcagaatggaggggtttgtgtggatggggtcaacacttacaactgccgctgtcccccacaatggac
aggacagttctgcacagaggatgtggatgaatgcctgctgcagcccaatgcctgtcaaaatgggggca
cctgtgccaaccgcaatggaggctatggctgtgtatgtgtcaacggctggagtggagatgactgcagt
gagaacattgatgattgtgccttcgcctcctgtactccaggctccacctgcatcgaccgtgtggcctc
cttctcttgcatgtgcccagaggggaaggcaggtctcctgtgtcatctggatgatgcatgcatcagca
atccttgccacaagggggcactgtgtgacaccaaccccctaaatgggcaatatatttgcacctgccca
caaggctacaaaggggctgactgcacagaagatgtggatgaatgtgccatggccaatagcaatccttg
tgagcatgcaggaaaatgtgtgaacacggatggcgccttccactgtgagtgtctgaagggttatgcag
gacctcgttgtgagatggacatcaatgagtgccattcagacccctgccagaatgatgctacctgtctg
gataagattggaggcttcacatgtctgtgcatgccaggtttcaaaggtgtgcattgtgaattagaaat
aaatgaatgtcagagcaacccttgtgtgaacaatgggcagtgtgtggataaagtcaatcgtttccagt
gcctgtgtcctcctggtttcactgggccagtttgccagattgatattgatgactgttccagtactccg
tgtctgaatggggcaaagtgtatcgatcacccgaatggctatgaatgccagtgtgccacaggtttcac
tggtgtgttgtgtgaggagaacattgacaactgtgaccccgatccttgccaccatggtcagtgtcagg
atggtattgattcctacacctgcatctgcaatcccgggtacatgggcgccatctgcagtgaccagatt
gatgaatgttacagcagcccttgcctgaacgatggtcgctgcattgacctggtcaatggctaccagtg
caactgccagccaggcacgtcaggggttaattgtgaaattaattttgatgactgtgcaagtaaccctt
gtatccatggaatctgtatggatggcattaatcgctacagttgtgtctgctcaccaggattcacaggg
cagagatgtaacattgacattgatgagtgtgcctccaatccctgtcgcaagggtgcaacatgtatcaa
cggtgtgaatggtttccgctgtatatgccccgagggaccccatcaccccagctgctactcacaggtga
acgaatgcctgagcaatccctgcatccatggaaactgtactggaggtctcagtggatataagtgtctc
tgtgatgcaggctgggttggcatcaactgtgaagtggacaaaaatgaatgcctttcgaatccatgcca
gaatggaggaacttgtgacaatctggtgaatggatacaggtgtacttgcaagaagggctttaaaggct
ataactgccaggtgaatattgatgaatgtgcctcaaatccatgcctgaaccaaggaacctgctttgat
gacataagtggctacacttgccactgtgtgctgccatacacaggcaagaattgtcagacagtattggc
tccctgttccccaaacccttgtgagaatgctgctgtttgcaaagagtcaccaaattttgagagttata
cttgcttgtgtgctcctggctggcaaggtcagcggtgtaccattgacattgacgagtgtatctccaag
ccctgcatgaaccatggtctctgccataacacccagggcagctacatgtgtgaatgtccaccaggctt
cagtggtatggactgtgaggaggacattgatgactgccttgccaatccttgccagaatggaggttcct
gtatggatggagtgaatactttctcctgcctctgccttccgggtttcactggggataagtgccagaca
gacatgaatgagtgtctgagtgaaccctgtaagaatggagggacctgctctgactacgtcaacagtta
cacttgcaagtgccaggcaggatttgatggagtccattgtgagaacaacatcaatgagtgcactgaga
gctcctgtttcaatggtggcacatgtgttgatgggattaactccttctcttgcttgtgccctgtgggt
ttcactggatccttctgcctccatgagatcaatgaatgcagctctcatccatgcctgaatgagggaac
gtgtgttgatggcctgggtacctaccgctgcagctgccccctgggctacactgggaaaaactgtcaga
ccctggtgaatctctgcagtcggtctccatgtaaaaacaaaggtacttgcgttcagaaaaaagcagag
tcccagtgcctatgtccatctggatgggctggtgcctattgtgacgtgcccaatgtctcttgtgacat
agcagcctccaggagaggtgtgcttgttgaacacttgtgccagcactcaggtgtctgcatcaatgctg
gcaacacgcattactgtcagtgccccctgggctatactgggagctactgtgaggagcaactcgatgag
tgtgcgtccaacccctgccagcacggggcaacatgcagtgacttcattggtggatacagatgcgagtg
tgtcccaggctatcagggtgtcaactgtgagtatgaagtggatgagtgccagaatcagccctgccaga
atggaggcacctgtattgaccttgtgaaccatttcaagtgctcttgcccaccaggcactcggggccta
ctctgtgaagagaacattgatgactgtgcccggggtccccattgccttaatggtggtcagtgcatgga
taggattggaggctacagttgtcgctgcttgcctggctttgctggggagcgttgtgagggagacatca
acgagtgcctctccaacccctgcagctctgagggcagcctggactgtatacagctcaccaatgactac
ctgtgtgtttgccgtagtgcctttactggccggcactgtgaaaccttcgtcgatgtgtgtccccagat
gccctgcctgaatggagggacttgtgctgtggccagtaacatgcctgatggtttcatttgccgttgtc
ccccgggattttccggggcaaggtgccagagcagctgtggacaagtgaaatgtaggaagggggagcag
tgtgtgcacaccgcctctggaccccgctgcttctgccccagtccccgggactgcgagtcaggctgtgc
cagtagcccctgccagcacgggggcagctgccaccctcagcgccagcctccttattactcctgccagt
gtgccccaccattctcgggtagccgctgtgaactctacacggcaccccccagcacccctcctgccacc
tgtctgagccagtattgtgccgacaaagctcgggatggcgtctgtgatgaggcctgcaacagccatgc
ctgccagtgggatgggggtgactgttctctcaccatggagaacccctgggccaactgctcctccccac
ttccctgctgggattatatcaacaaccagtgtgatgagctgtgcaacacggtcgagtgcctgtttgac
aactttgaatgccaggggaacagcaagacatgcaagtatgacaaatactgtgcagaccacttcaaaga
caaccactgtgaccaggggtgcaacagtgaggagtgtggttgggatgggctggactgtgctgctgacc
aacctgagaacctggcagaaggtaccctggttattgtggtattgatgccacctgaacaactgctccag
gatgctcgcagcttcttgcgggcactgggtaccctgctccacaccaacctgcgcattaagcgggactc
ccagggggaactcatggtgtacccctattatggtgagaagtcagctgctatgaagaaacagaggatga
cacgcagatcccttcctggtgaacaagaacaggaggtggctggctctaaagtctttctggaaattgac
aaccgccagtgtgttcaagactcagaccactgcttcaagaacacggatgcagcagcagctctcctggc
ctctcacgccatacaggggaccctgtcataccctcttgtgtctgtcgtcagtgaatccctgactccag
aacgcactcagctcctctatctccttgctgttgctgttgtcatcattctgtttattattctgctgggg
gtaatcatggcaaaacgaaagcgtaagcatggctctctctggctgcctgaaggtttcactcttcgccg
agatgcaagcaatcacaagcgtcgtgagccagtgggacaggatgctgtggggctgaaaaatctctcag
tgcaagtctcagaagctaacctaattggtactggaacaagtgaacactgggtcgatgatgaagggccc
cagccaaagaaagtaaaggctgaagatgaggccttactctcagaagaagatgaccccattgatcgacg
gccatggacacagcagcaccttgaagctgcagacatccgtaggacaccatcgctggctctcacccctc
ctcaggcagagcaggaggtggatgtgttagatgtgaatgtccgtggcccagatggctgcaccccattg
atgttggcttctctccgaggaggcagctcagatttgagtgatgaagatgaagatgcagaggactcttc
tgctaacatcatcacagacttggtctaccagggtgccagcctccaggcccagacagaccggactggtg
agatggccctgcaccttgcagcccgctactcacgggctgatgctgccaagcgtctcctggatgcaggt
gcagatgccaatgcccaggacaacatgggccgctgtccactccatgctgcagtggcagctgatgccca
aggtgtcttccagattctgattcgcaaccgagtaactgatctagatgccaggatgaatgatggtacta
cacccctgatcctggctgcccgcctggctgtggagggaatggtggcagaactgatcaactgccaagcg
gatgtgaatgcagtggatgaccatggaaaatctgctcttcactgggcagctgctgtcaataatgtgga
ggcaactcttttgttgttgaaaaatggggccaaccgagacatgcaggacaacaaggaagagacacctc
tgtttcttgctgcccgggaggggagctatgaagcagccaagatcctgttagaccattttgccaatcga
gacatcacagaccatatggatcgtcttccccgggatgtggctcgggatcacatgcaccatgacattgt
gcgccttctggatgaatacaatgtgaccccaagccctccaggcaccgtgttgacttctgctctctcac
ctgtcatctgtgggcccaacagatctttcctcagcctgaagcacaccccaatgggcaagaagtctaga
cggcccagtgccaagagtaccatgcctactagcctccctaaccttgccaaggaggcaaaggatgccaa
gggtagtaggaggaagaagtctctgagtgagaaggtccaactgtctgagagttcagtaactttatccc
ctgttgattccctagaatctcctcacacgtatgtttccgacaccacatcctctccaatgattacatcc
cctgggatcttacaggcctcacccaaccctatgttggccactgccgcccctcctgccccagtccatgc
ccagcatgcactatctttttctaaccttcatgaaatgcagcctttggcacatggggccagcactgtgc
ttccctcagtgagccagttgctatcccaccaccacattgtgtctccaggcagtggcagtgctggaagc
ttgagtaggctccatccagtcccagtcccagcagattggatgaaccgcatggaggtgaatgagaccca
gtacaatgagatgtttggtatggtcctggctccagctgagggcacccatcctggcatagctccccaga
gcaggccacctgaagggaagcacataaccacccctcgggagcccttgccccccattgtgactttccag
ctcatccctaaaggcagtattgcccaaccagcgggggctccccagcctcagtccacctgccctccagc
tgttgcgggccccctgcccaccatgtaccagattccagaaatggcccgtttgcccagtgtggctttcc
ccactgccatgatgccccagcaggacgggcaggtagctcagaccattctcccagcctatcatcctttc
ccagcctctgtgggcaagtaccccacacccccttcacagcacagttatgcttcctcaaatgctgctga
gcgaacacccagtcacagtggtcacctccagggtgagcatccctacctgacaccatccccagagtctc
ctgaccagtggtcaagttcatcaccccactctgcttctgactggtcagatgtgaccaccagccctacc
cctgggggtgctggaggaggtcagcggggacctgggacacacatgtctgagccaccacacaacaacat
gcaggtttatgcgtgagagagtccacctccagtgtagagacataactgacttttgtaaatgctgctga
ggaacaaatgaaggtcatccgggagagaaatgaagaaatctctggagccagcttctagaggtaggaaa
gagaagatgttcttattcagataatgcaagagaagcaattcgtcagtttcactgggtatctgcaaggc
ttattgattattctaatctaataagacaagtttgtggaaatgcaagatgaatacaagccttgggtcca
tgtttactctcttctatttggagaataagatggatgcttattgaagcccagacattcttgcagcttgg
actgcattttaagccctgcaggcttctgccatatccatgagaagattctacactagcgtcctgttggg
aattatgccctggaattctgcctgaattgacctacgcatctcctcctccttggacattcttttgtctt
catttggtgcttttggttttgcacctctccgtgattgtagccctaccagcatgttatagggcaagacc
tttgtgcttttgatcattctggcccatgaaagcaactttggtctcctttcccctcctgtcttcccggt
atcccttggagtctcacaaggtttactttggtatggttctcagcacaaacctttcaagtatgttgttt
ctttggaaaatggacatactgtattgtgttctcctgcatatatcattcctggagagagaaggggagaa
gaatacttttcttcaacaaattttgggggcaggagatcccttcaagaggctgcaccttaatttttctt
gtctgtgtgcaggtcttcatataaactttaccaggaagaagggtgtgagtttgttgtttttctgtgta
tgggcctggtcagtgtaaagttttatccttgatagtctagttactatgaccctccccacttttttaaa
accagaaaaaggtttggaatgttggaatgaccaagagacaagttaactcgtgcaagagccagttaccc
acccacaggtccccctacttcctgccaagcattccattgactgcctgtatggaacacatttgtcccag
atctgagcattctaggcctgtttcactcactcacccagcatatgaaactagtcttaactgttgagcct
ttcctttcatatccacagaagacactgtctcaaatgttgtacccttgccatttaggactgaactttcc
ttagcccaagggacccagtgacagttgtcttccgtttgtcagatgatcagtctctactgattatcttg
ctgcttaaaggcctgctcaccaatctttctttcacaccgtgtggtccgtgttactggtatacccagta
tgttctcactgaagacatggactttatatgttcaagtgcaggaattggaaagttggacttgttttcta
tgatccaaaacagccctataagaaggttggaaaaggaggaactatatagcagcctttgctattttctg
ctaccatttcttttcctctgaagcggccatgacattccctttggcaactaacgtagaaactcaacaga
acattttcctttcctagagtcaccttttagatgataatggacaactatagacttgctcattgttcaga
ctgattgcccctcacctgaatccactctctgtattcatgctcttggcaatttctttgactttctttta
agggcagaagcattttagttaattgtagataaagaatagttttcttcctcttctccttgggccagtta
ataattggtccatggctacactgcaacttccgtccagtgctgtgatgcccatgacacctgcaaaataa
gttctgcctgggcattttgtagatattaacaggtgaattcccgactcttttggtttgaatgacagttc
tcattccttctatggctgcaagtatgcatcagtgcttcccacttacctgatttgtctgtcggtggccc
catatggaaaccctgcgtgtctgttggcataatagtttacaaatggttttttcagtcctatccaaatt
tattgaaccaacaaaaataattacttctgccctgagataagcagattaagtttgttcattctctgctt
tattctctccatgtggcaacattctgtcagcctctttcatagtgtgcaaacattttatcattctaaat
ggtgactctctgcccttggacccatttattattcacagatggggagaacctatctgcatggacctctg
tggaccacagcgtacctgcccctttctgccctcctgctccagccccacttctgaaagtatcagctact
gatccagccactggatattttatatcctcccttttccttaagcacaatgtcagaccaaattgcttgtt
tctttttcttggactactttaatttggatcctttgggtttggagaaagggaatgtgaaagctgtcatt
acagacaacaggtttcagtgatgaggaggacaacactgcctttcaaactttttactgatctcttagat
tttaagaactcttgaattgtgtggtatctaataaaagggaaggtaagatggataatcactttctcatt
tgggttctgaattggagactcagtttttatgagacacatcttttatgccatgtatagatcctcccctg
ctatttttggtttatttttattgttataaatgctttctttctttgactcctcttctgcctgcctttgg
ggataggtttttttgtttgtttatttgcttcctctgttttgttttaagcatcattttcttatgtgagg
tggggaagggaaaggtatgagggaaagagagtctgagaattaaaatattttagtataagcaattggct
gtgatgctcaaatccattgcatcctcttattgaatttgccaatttgtaatttttgcataataaagaac
caaaggtgtaatgttttgttgagaggtggtttagggattttggccctaaccaatacattgaatgtatg
atgactatttgggaggacacatttatgtacccagaggcccccactaataagtggtactatggttactt
ccttgtgtacatttctcttaaaagtgatattatatctgtttgtatgagaaacccagtaaccaataaaa
tgaccgcatattcctgactaaacgtagtaaggaaaatgcacactttgtttttacttttccgtttcatt
ctaaaggtagttaagatgaaatttatatgaaagcatttttatcacaaaataaaaaaggtttgccaagc
tcagtggtgttgtattttttattttccaatactgcatccatggcctggcagtgttacctcatgatgtc
ataatttgctgagagagcaaattttcttttctttctgaatcccacaaagcctagcaccaaacttcttt
ttttcttcctttaattagatcataaataaatgatcctggggaaaaagcatctgtcaaataggaaacat
cacaaaactgagcactcttctgtgcactagccatagctggtgacaaacagatggttgctcagggacaa
ggtgccttccaatggaaatgcgaagtagttgctatagcaagaattgggaactgggatataagtcataa
tattaattatgctgttatgtaaatgattggtttgtaacattccttaagtgaaatttgtgtagaactta
atatacaggattataaaataatattttgtgtataaatttgttataagttcacattcatacatttattt
ataaagtcagtgagatatttgaacatgaaaaaaaaaa
Human NOTCH3 gene (wild-type)
SEQ ID NO: 3
gcggcgcggaggctggcccgggacgcgcccggagcccagggaaggagggaggaggggagggtcgcggc
cggccgccatggggccgggggcccgtggccgccgccgccgccgtcgcccgatgtcgccgccaccgcca
ccgccacccgtgcgggcgctgcccctgctgctgctgctagcggggccgggggctgcagcccccccttg
cctggacggaagcccgtgtgcaaatggaggtcgttgcacccagctgccctcccgggaggctgcctgcc
tgtgcccgcctggctgggtgggtgagcggtgtcagctggaggacccctgtcactcaggcccctgtgct
ggccgtggtgtctgccagagttcagtggtggctggcaccgcccgattctcatgccggtgcccccgtgg
cttccgaggccctgactgctccctgccagatccctgcctcagcagcccttgtgcccacggtgcccgct
gctcagtggggcccgatggacgcttcctctgctcctgcccacctggctaccagggccgcagctgccga
agcgacgtggatgagtgccgggtgggtgagccctgccgccatggtggcacctgcctcaacacacctgg
ctccttccgctgccagtgtccagctggctacacagggccactatgtgagaaccccgcggtgccctgtg
caccctcaccatgccgtaacgggggcacctgcaggcagagtggcgacctcacttacgactgtgcctgt
cttcctgggtttgagggtcagaattgtgaagtgaacgtggacgactgtccaggacaccgatgtctcaa
tggggggacatgcgtggatggcgtcaacacctataactgccagtgccctcctgagtggacaggccagt
tctgcacggaggacgtggatgagtgtcagctgcagcccaacgcctgccacaatgggggtacctgcttc
aacacgctgggtggccacagctgcgtgtgtgtcaatggctggacaggcgagagctgcagtcagaatat
cgatgactgtgccacagccgtgtgcttccatggggccacctgccatgaccgcgtggcttctttctact
gtgcctgccccatgggcaagactggcctcctgtgtcacctggatgacgcctgtgtcagcaacccctgc
cacgaggatgctatctgtgacacaaatccggtgaacggccgggccatttgcacctgtcctcccggctt
cacgggtggggcatgtgaccaggatgtggacgagtgctctatcggcgccaacccctgcgagcacttgg
gcaggtgcgtgaacacgcagggctccttcctgtgccagtgcggtcgtggctacactggacctcgctgt
gagaccgatgtcaacgagtgtctgtcggggccctgccgaaaccaggccacgtgcctcgaccgcatagg
ccagttcacctgtatctgtatggcaggcttcacaggaacctattgcgaggtggacattgacgagtgtc
agagtagcccctgtgtcaacggtggggtctgcaaggaccgagtcaatggcttcagctgcacctgcccc
tcgggcttcagcggctccacgtgtcagctggacgtggacgaatgcgccagcacgccctgcaggaatgg
cgccaaatgcgtggaccagcccgatggctacgagtgccgctgtgccgagggctttgagggcacgctgt
gtgatcgcaacgtggacgactgctcccctgacccatgccaccatggtcgctgcgtggatggcatcgcc
agcttctcatgtgcctgtgctcctggctacacgggcacacgctgcgagagccaggtggacgaatgccg
cagccagccctgccgccatggcggcaaatgcctagacctggtggacaagtacctctgccgctgccctt
ctgggaccacaggtgtgaactgcgaagtgaacattgacgactgtgccagcaacccctgcacctttgga
gtctgccgtgatggcatcaaccgctacgactgtgtctgccaacctggcttcacagggcccctttgtaa
cgtggagatcaatgagtgtgcttccagcccatgcggcgagggaggttcctgtgtggatggggaaaatg
gcttccgctgcctctgcccgcctggctccttgcccccactctgcctccccccgagccatccctgtgcc
catgagccctgcagtcacggcatctgctatgatgcacctggcgggttccgctgtgtgtgtgagcctgg
ctggagtggcccccgctgcagccagagcctggcccgagacgcctgtgagtcccagccgtgcagggccg
gtgggacatgcagcagcgatggaatgggtttccactgcacctgcccgcctggtgtccagggacgtcag
tgtgaactcctctccccctgcaccccgaacccctgtgagcatgggggccgctgcgagtctgcccctgg
ccagctgcctgtctgctcctgcccccagggctggcaaggcccacgatgccagcaggatgtggacgagt
gtgctggccccgcaccctgtggccctcatggtatctgcaccaacctggcagggagtttcagctgcacc
tgccatggagggtacactggcccttcctgcgatcaggacatcaatgactgtgaccccaacccatgcct
gaacggtggctcgtgccaagacggcgtgggctccttttcctgctcctgcctccctggtttcgccggcc
cacgatgcgcccgcgatgtggatgagtgcctgagcaacccctgcggcccgggcacctgtaccgaccac
gtggcctccttcacctgcacctgcccgccaggctacggaggcttccactgcgaacaggacctgcccga
ctgcagccccagctcctgcttcaatggcgggacctgtgtggacggcgtgaactcgttcagctgcctgt
gccgtcccggctacacaggagcccactgccaacatgaggcagacccctgcctctcgcggccctgccta
cacgggggcgtctgcagcgccgcccaccctggcttccgctgcacctgcctcgagagcttcacgggccc
gcagtgccagacgctggtggattggtgcagccgccagccttgtcaaaacgggggtcgctgcgtccaga
ctggggcctattgcctttgtccccctggatggagcggacgcctctgtgacatccgaagcttgccctgc
agggaggccgcagcccagatcggggtgcggctggagcagctgtgtcaggcgggtgggcagtgtgtgga
tgaagacagctcccactactgcgtgtgcccagagggccgtactggtagccactgtgagcaggaggtgg
acccctgcttggcccagccctgccagcatggggggacctgccgtggctatatggggggctacatgtgt
gagtgtcttcctggctacaatggtgataactgtgaggacgacgtggacgagtgtgcctcccagccctg
ccagcacgggggttcatgcattgacctcgtggcccgctatctctgctcctgtcccccaggaacgctgg
gggtgctctgcgagattaatgaggatgactgcggcccaggcccaccgctggactcagggccccggtgc
ctacacaatggcacctgcgtggacctggtgggtggtttccgctgcacctgtcccccaggatacactgg
tttgcgctgcgaggcagacatcaatgagtgtcgctcaggtgcctgccacgcggcacacacccgggact
gcctgcaggacccaggcggaggtttccgttgcctttgtcatgctggcttctcaggtcctcgctgtcag
actgtcctgtctccctgcgagtcccagccatgccagcatggaggccagtgccgtcctagcccgggtcc
tgggggtgggctgaccttcacctgtcactgtgcccagccgttctggggtccgcgttgcgagcgggtgg
cgcgctcctgccgggagctgcagtgcccggtgggcgtcccatgccagcagacgccccgcgggccgcgc
tgcgcctgccccccagggttgtcgggaccctcctgccgcagcttcccggggtcgccgccgggggccag
caacgccagctgcgcggccgccccctgtctccacgggggctcctgccgccccgcgccgctcgcgccct
tcttccgctgcgcttgcgcgcagggctggaccgggccgcgctgcgaggcgcccgccgcggcacccgag
gtctcggaggagccgcggtgcccgcgcgccgcctgccaggccaagcgcggggaccagcgctgcgaccg
cgagtgcaacagcccaggctgcggctgggacggcggcgactgctcgctgagcgtgggcgacccctggc
ggcaatgcgaggcgctgcagtgctggcgcctcttcaacaacagccgctgcgaccccgcctgcagctcg
cccgcctgcctctacgacaacttcgactgccacgccggtggccgcgagcgcacttgcaacccggtgta
cgagaagtactgcgccgaccactttgccgacggccgctgcgaccagggctgcaacacggaggagtgcg
gctgggatgggctggattgtgccagcgaggtgccggccctgctggcccgcggcgtgctggtgctcaca
gtgctgctgccgccagaggagctactgcgttccagcgccgactttctgcagcggctcagcgccatcct
gcgcacctcgctgcgcttccgcctggacgcgcacggccaggccatggtcttcccttaccaccggccta
gtcctggctccgaaccccgggcccgtcgggagctggcccccgaggtgatcggctcggtagtaatgctg
gagattgacaaccggctctgcctgcagtcgcctgagaatgatcactgcttccccgatgcccagagcgc
cgctgactacctgggagcgttgtcagcggtggagcgcctggacttcccgtacccactgcgggacgtgc
ggggggagccgctggagcctccagaacccagcgtcccgctgctgccactgctagtggcgggcgctgtc
ttgctgctggtcattctcgtcctgggtgtcatggtggcccggcgcaagcgcgagcacagcaccctctg
gttccctgagggcttctcactgcacaaggacgtggcctctggtcacaagggccggcgggaacccgtgg
gccaggacgcgctgggcatgaagaacatggccaagggtgagagcctgatgggggaggtggccacagac
tggatggacacagagtgcccagaggccaagcggctaaaggtagaggagccaggcatgggggctgagga
ggctgtggattgccgtcagtggactcaacaccatctggttgctgctgacatccgcgtggcaccagcca
tggcactgacaccaccacagggcgacgcagatgctgatggcatggatgtcaatgtgcgtggcccagat
ggcttcaccccgctaatgctggcttccttctgtgggggggctctggagccaatgccaactgaagagga
tgaggcagatgacacatcagctagcatcatctccgacctgatctgccagggggctcagcttggggcac
ggactgaccgtactggcgagactgctttgcacctggctgcccgttatgcccgtgctgatgcagccaag
cggctgctggatgctggggcagacaccaatgcccaggaccactcaggccgcactcccctgcacacagc
tgtcacagccgatgcccagggtgtcttccagattctcatccgaaaccgctctacagacttggatgccc
gcatggcagatggctcaacggcactgatcctggcggcccgcctggcagtagagggcatggtggaagag
ctcatcgccagccatgctgatgtcaatgctgtggatgagcttgggaaatcagccttacactgggctgc
ggctgtgaacaacgtggaagccactttggccctgctcaaaaatggagccaataaggacatgcaggata
gcaaggaggagacccccctattcctggccgcccgcgagggcagctatgaggctgccaagctgctgttg
gaccactttgccaaccgtgagatcaccgaccacctggacaggctgccgcgggacgtagcccaggagag
actgcaccaggacatcgtgcgcttgctggatcaacccagtgggccccgcagcccccccggtccccacg
gcctggggcctctgctctgtcctccaggggccttcctccctggcctcaaagcggcacagtcggggtcc
aagaagagcaggaggccccccgggaaggcggggctggggccgcaggggccccgggggcggggcaagaa
gctgacgctggcctgcccgggccccctggctgacagctcggtcacgctgtcgcccgtggactcgctgg
actccccgcggcctttcggtgggccccctgcttcccctggtggcttcccccttgaggggccctatgca
gctgccactgccactgcagtgtctctggcacagcttggtggcccaggccgggcgggtctagggcgcca
gccccctggaggatgtgtactcagcctgggcctgctgaaccctgtggctgtgcccctcgattgggccc
ggctgcccccacctgcccctccaggcccctcgttcctgctgccactggcgccgggaccccagctgctc
aacccagggacccccgtctccccgcaggagcggcccccgccttacctggcagtcccaggacatggcga
ggagtacccggcggctggggcacacagcagccccccaaaggcccgcttcctgcgggttcccagtgagc
acccttacctgaccccatcccccgaatcccctgagcactgggccagcccctcacctccctccctctca
gactggtccgaatccacgcctagcccagccactgccactggggccatggccaccaccactggggcact
gcctgcccagccacttcccttgtctgttcccagctcccttgctcaggcccagacccagctggggcccc
agccggaagttacccccaagaggcaagtgttggcctgagacgctcgtcagttcttagatcttgggggc
ctaaagagacccccgtcctgcctcctttctttctctgtctcttccttccttttagtctttttcatcct
cttctctttccaccaaccctcctgcatccttgccttgcagcgtgaccgagataggtcatcagcccagg
gcttcagtcttcctttatttataatgggtgggggctaccacccaccctctcagtcttgtgaagagtct
gggacctccttcttccccacttctctcttccctcattcctttctctctccttctggcctctcatttcc
ttacactctgacatgaatgaattattattatttttatttttctttttttttttacattttgtatagaa
acaaattcatttaaacaaacttattattattattttttacaaaatatatatatggagatgctccctcc
ccctgtgaaccccccagtgcccccgtggggctgagtctgtgggcccattcggccaagctggattctgt
gtacctagtacacaggcatgactgggatcccgtgtaccgagtacacgacccaggtatgtaccaagtag
gcacccttgggcgcacccactggggccaggggtcgggggagtgttgggagcctcctccccaccccacc
tccctcacttcactgcattccagatgggacatgttccatagccttgctggggaagggcccactgccaa
ctccctctgccccagccccacccttggccatctccctttgggaactagggggctgctggtgggaaatg
ggagccagggcagatgtatgcattcctttgtgtccctgtaaatgtgggactacaagaagaggagctgc
ctgagtggtactttctcttcctggtaatcctctggcccagcctcatggcagaatagaggtatttttag
gctatttttgtaatatggcttctggtcaaaatccctgtgtagctgaattcccaagccctgcattgtac
agccccccactcccctcaccacctaataaaggaatagttaacactcaaaaaaaaaaaaaaaaaaa
Human NOTCH4 gene (wild-type)
SEQ ID NO: 4
agacgtgaggcttgcagcaggccgaggaggaagaagaggggcagtgggagcagaggaggtggctcctg
ccccagtgagagctctgagggtccctgcctgaagagggacagggaccggggcttggagaaggggctgt
ggaatgcagcccccttcactgctgctgctgctgctgctgctgctgctgctatgtgtctcagtggtcag
acccagagggctgctgtgtgggagtttcccagaaccctgtgccaatggaggcacctgcctgagcctgt
ctctgggacaagggacctgccagtgtgcccctggcttcctgggtgagacgtgccagtttcctgacccc
tgccagaacgcccagctctgccaaaatggaggcagctgccaagccctgcttcccgctcccctagggct
ccccagctctccctctccattgacacccagcttcttgtgcacttgcctccctggcttcactggtgaga
gatgccaggccaagcttgaagacccttgtcctccctccttctgttccaaaaggggccgctgccacatc
caggcctcgggccgcccacagtgctcctgcatgcctggatggacaggtgagcagtgccagcttcggga
cttctgttcagccaacccatgtgttaatggaggggtgtgtctggccacatacccccagatccagtgcc
actgcccaccgggcttcgagggccatgcctgtgaacgtgatgtcaacgagtgcttccaggacccagga
ccctgccccaaaggcacctcctgccataacaccctgggctccttccagtgcctctgccctgtggggca
ggagggtccacgttgtgagctgcgggcaggaccctgccctcctaggggctgttcgaatgggggcacct
gccagctgatgccagagaaagactccacctttcacctctgcctctgtcccccaggtttcataggccca
gactgtgaggtgaatccagacaactgtgtcagccaccagtgtcagaatgggggcacttgccaggatgg
gctggacacctacacctgcctctgcccagaaacctggacaggctgggactgctccgaagatgtggatg
agtgtgagacccagggtccccctcactgcagaaacgggggcacctgccagaactctgctggtagcttt
cactgcgtgtgtgtgagtggctggggcggcacaagctgtgaggagaacctggatgactgtattgctgc
cacctgtgccccgggatccacctgcattgaccgggtgggctctttctcctgcctctgcccacctggac
gcacaggactcctgtgccacttggaagacatgtgtctgagccagccgtgccatggggatgcccaatgc
agcaccaaccccctcacaggctccacactctgcctgtgtcagcctggctattcggggcccacctgcca
ccaggacctggacgagtgtctgatggcccagcaaggcccaagtccctgtgaacatggcggttcctgcc
tcaacactcctggctccttcaactgcctctgtccacctggctacacaggctcccgttgtgaggctgat
cacaatgagtgcctctcccagccctgccacccaggaagcacctgtctggacctacttgccaccttcca
ctgcctctgcccgccaggcttagaagggcagctctgtgaggtggagaccaacgagtgtgcctcagctc
cctgcctgaaccacgcggattgccatgacctgctcaacggcttccagtgcatctgcctgcctggattc
tccggcacccgatgtgaggaggatatcgatgagtgcagaagctctccctgtgccaatggtgggcagtg
ccaggaccagcctggagccttccactgcaagtgtctcccaggctttgaagggccacgctgtcaaacag
aggtggatgagtgcctgagtgacccatgtcccgttggagccagctgccttgatcttccaggagccttc
ttttgcctctgcccctctggtttcacaggccagctctgtgaggttcccctgtgtgctcccaacctgtg
ccagcccaagcagatatgtaaggaccagaaagacaaggccaactgcctctgtcctgatggaagccctg
gctgtgccccacctgaggacaactgcacctgccaccacgggcactgccagagatcctcatgtgtgtgt
gacgtgggttggacggggccagagtgtgaggcagagctagggggctgcatctctgcaccctgtgccca
tggggggacctgctacccccagccctctggctacaactgcacctgccctacaggctacacaggaccca
cctgtagtgaggagatgacagettgtcactcagggccatgtctcaatggcggctcctgcaaccctage
cctggaggctactactgcacctgccctccaagccacacagggccccagtgccaaaccagcactgacta
ctgtgtgtctgccccgtgcttcaatgggggtacctgtgtgaacaggcctggcaccttctcctgcctct
gtgccatgggcttccagggcccgcgctgtgagggaaagctccgccccagctgtgcagacagcccctgt
aggaatagggcaacctgccaggacagccctcagggtccccgctgcctctgccccactggctacaccgg
aggcagctgccagactctgatggacttatgtgcccagaagccctgcccacgcaattcccactgcctcc
agactgggccctccttccactgcttgtgcctccagggatggaccgggcctctctgcaaccttccactg
tcctcctgccagaaggctgcactgagccaaggcatagacgtctcttccctttgccacaatggaggcct
ctgtgtcgacagcggcccctcctatttctgccactgcccccctggattccaaggcagcctgtgccagg
atcacgtgaacccatgtgagtccaggccttgccagaacggggccacctgcatggcccagcccagtggg
tatctctgccagtgtgccccaggctacgatggacagaactgctcaaaggaactcgatgcttgtcagtc
ccaaccctgtcacaaccatggaacctgtactcccaaacctggaggattccactgtgcctgccctccag
gctttgtggggctacgctgtgagggagacgtggacgagtgtctggaccagccctgccaccccacaggc
actgcagcctgccactctctggccaatgccttctactgccagtgtctgcctggacacacaggccagtg
gtgtgaggtggagatagacccctgccacagccaaccctgctttcatggagggacctgtgaggccacag
caggatcacccctgggtttcatctgccactgccccaagggttttgaaggccccacctgcagccacagg
gccccttcctgcggcttccatcactgccaccacggaggcctgtgtctgccctcccctaagccaggctt
cccaccacgctgtgcctgcctcagtggctatgggggtcctgactgcctgaccccaccagctcctaaag
gctgtggccctccctccccatgcctatacaatggcagctgctcagagaccacgggcttggggggccca
ggctttcgatgctcctgccctcacagctctccagggccccggtgtcagaaacccggagccaaggggtg
tgagggcagaagtggagatggggcctgcgatgctggctgcagtggcccgggaggaaactgggatggag
gggactgctctctgggagtcccagacccctggaagggctgcccctcccactctcggtgctggcttctc
ttccgggacgggcagtgccacccacagtgtgactctgaagagtgtctgtttgatggctacgactgtga
gacccctccagcctgcactccagcctatgaccagtactgccatgatcacttccacaacgggcactgtg
agaaaggctgcaacactgcagagtgtggctgggatggaggtgactgcaggcctgaagatggggaccca
gagtgggggccctccctggccctgctggtggtactgagccccccagccctagaccagcagctgtttgc
cctggcccgggtgctgtccctgactctgagggtaggactctgggtaaggaaggatcgtgatggcaggg
acatggtgtacccctatcctggggcccgggctgaagaaaagctaggaggaactcgggaccccacctat
caggagagagcagcccctcaaacgcagcccctgggcaaggagaccgactccctcagtgctgggtttgt
ggtggtcatgggtgtggatttgtcccgctgtggccctgaccacccggcatcccgctgtccctgggacc
ctgggcttctactccgcttccttgctgcgatggctgcagtgggagccctggagcccctgctgcctgga
ccactgctggctgtccaccctcatgcagggaccgcaccccctgccaaccagcttccctggcctgtgct
gtgctccccagtggccggggtgattctcctggccctaggggctcttctcgtcctccagctcatccggc
gtcgacgccgagagcatggagctctctggctgccccctggtttcactcgacggcctcggactcagtca
gctccccaccgacgccggcccccactaggcgaggacagcattggtctcaaggcactgaagccaaaggc
agaagttgatgaggatggagttgtgatgtgctcaggccctgaggagggagaggaggtgggccaggctg
aagaaacaggcccaccctccacgtgccagctctggtctctgagtggtggctgtggggcgctccctcag
gcagccatgctaactcctccccaggaatctgagatggaagcccctgacctggacacccgtggacctga
tggggtgacacccctgatgtcagcagtttgctgtggggaagtacagtccgggaccttccaaggggcat
ggttgggatgtcctgagccctgggaacctctgctggatggaggggcctgtccccaggctcacaccgtg
ggcactggggagacccccctgcacctggctgcccgattctcccggccaaccgctgcccgccgcctcct
tgaggctggagccaaccccaaccagccagaccgggcagggcgcacaccccttcatgctgctgtggctg
ctgatgctcgggaggtctgccagcttctgctccgtagcagacaaactgcagtggacgctcgcacagag
gacgggaccacacccttgatgctggctgccaggctggcggtggaagacctggttgaagaactgattgc
agcccaagcagacgtgggggccagagataaatgggggaaaactgcgctgcactgggctgctgccgtga
acaacgcccgagccgcccgctcgcttctccaggccggagccgataaagatgcccaggacaacagggag
cagacgccgctattcctggcggcgcgggaaggagcggtggaagtagcccagctactgctggggctggg
ggcagcccgagagctgcgggaccaggctgggctagcgccggcggacgtcgctcaccaacgtaaccact
gggatctgctgacgctgctggaaggggctgggccaccagaggcccgtcacaaagccacgccgggccgc
gaggctgggcccttcccgcgcgcacggacggtgtcagtaagcgtgcccccgcatgggggcggggctct
gccgcgctgccggacgctgtcagccggagcaggccctcgtgggggcggagcttgtctgcaggctcgga
cttggtccgtagacttggctgcgcgggggggcggggcctattctcattgccggagcctctcgggagta
ggagcaggaggaggcccgacccctcgcggccgtaggttttctgcaggcatgcgcgggcctcggcccaa
ccctgcgataatgcgaggaagatacggagtggctgccgggcgcggaggcagggtctcaacggatgact
ggccctgtgattgggtggccctgggagcttgcggttctgcctccaacattccgatcccgcctccttgc
cttactccgtccccggagcggggatcacctcaacttgactgtggtcccccagccctccaagaaatgcc
cataaaccaaggaggagagggtaaaaaatagaagaatacatggtagggaggaattccaaaaatgatta
cccattaaaaggcaggctggaaggccttcctggttttaagatggatcccccaaaatgaagggttgtga
gtttagtttctctcctaaaatgaatgtatgcccaccagagcagacatcttccacgtggagaagctgca
gctctggaaagagggtttaagatgctaggatgaggcaggcccagtcctcctccagaaaataagacagg
ccacaggagggcagagtggagtggaaatacccctaagttggaaccaagaattgcaggcatatgggatg
taagatgttctttcctatatatggtttccaaagggtgcccctatgatccattgtccccactgcccaca
aatggctgacaaatatttattgggcacctactatgtgccaggcactgtgtaggtgctgaaaagtggcc
aagggccacccccgctgatgactccttgcattccctcccctcacaacaaagaactccactgtggggat
gaagcgcttcttctagccactgctatcgctatttaagaaccctaaatctgtcacccataataaagctg
atttgaagtgttaaaaaaaaaaaaaaaaaa
52M51 Heavy chain CDR1
SEQ ID NO: 5
RGYWIE
52M51 Heavy chain CDR2
SEQ ID NO: 6
QILPGTGRTNYNEKFKG
52M51 Heavy chain CDR3
SEQ ID NO: 7
FDGNYGYYAMDY
52M51 Light chain CDR1
SEQ ID NO: 8
RSSTGAVTTSNYAN
52M51 Light chain CDR2
SEQ ID NO: 9
GTNNRAP
52M51 Light chain CDR3
SEQ ID NO: 10
ALWYSNHWVFGGGTKL
Humanized 52M51 sequences:
52M51-H4 Heavy chain polynucleotide sequence (Putative signal
sequence underlined)
SEQ ID NO: 11
ATGGATTGGACATGGAGGGTGTTCTGCCTCCTCGCTGTGGCTCCTGGAGTCCTGAGCCAG
GTCCAGCTCGTCCAGAGCGGGGCTGAAGTCAAGAAGCCTGGCGCTAGCGTCAAAATCAGC
TGTAAGGTCAGCGGATACACACTGAGGGGATACTGGATCGAGTGGGTGAGGCAGGCTCCA
GGAAAGGGCCTGGAATGGATCGGCCAGATCCTGCCTGGAACCGGAAGGACAAATTACAAT
GAGAAGTTTAAGGGAAGGGTCACAATGACAGCAGACACAAGCACAGACACAGCTTATATG
GAACTCAGCTCCCTCAGATCCGAGGACACCGCTGTCTACTATTGTGCCAGGTTCGATGGA
AATTACGGATACTATGCCATGGATTACTGGGGACAGGGGACAACGGTCACCGTGAGCTCA
GCCAGCACAAAGGGCCCTAGCGTCTTCCCTCTGGCTCCCTGCAGCAGGAGCACCAGCGAG
AGCACAGCCGCCCTGGGCTGCCTGGTCAAGGACTACTTCCCCGAACCGGTGACGGTGTCG
TGGAACTCAGGCGCTCTGACCAGCGGCGTGCACACCTTCCCAGCTGTCCTACAGTCCTCA
GGACTCTACTCCCTCAGCAGCGTGGTGACCGTGCCCTCCAGCAACTTCGGCACCCAGACC
TACACCTGCAACGTAGATCACAAGCCCAGCAACACCAAGGTGGACAAGACAGTTGAGCGC
AAATGTTGTGTCGAGTGCCCACCGTGCCCAGCACCACCTGTGGCAGGACCGTCAGTCTTC
CTCTTCCCCCCAAAACCCAAGGACACCCTCATGATCTCCCGGACCCCTGAGGTCACGTGC
GTGGTGGTGGACGTGAGCCACGAAGACCCCGAGGTCCAGTTCAACTGGTACGTGGACGGC
GTGGAGGTGCATAATGCCAAGACAAAGCCACGGGAGGAGCAGTTCAACAGCACGTTCCGT
GTGGTCAGCGTCCTCACCGTTGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGG
CAGCCCCGAGAACCACAGGTGTACACCCTGCCCCCATCCCGGGAGGAGATGACCAAGAAC
CAGGTCAGCCTGACCTGCCTGGTCAAAGGCTTCTACCCCAGCGACATCGCCGTGGAGTGG
GAGAGCAATGGGCAGCCGGAGAACAACTACAAGACCACACCTCCCATGCTGGACTCCGAC
GGCTCCTTCTTCCTCTACAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTC
TCCCTGTCTCCGGGTAAATGA
52M51 H4 Heavy chain amino acid sequence (Putative signal sequence
underlined)
SEQ ID NO: 12
MDWTWRVFCLLAVAPGVLSQVQLVQSGAEVKKPGASVKISCKVSGYTLRGYWIEWVRQAP
GKGLEWIGQILPGTGRTNYNEKFKGRVTMTADTSTDTAYMELSSLRSEDTAVYYCARFDG
NYGYYAMDYWGQGTTVTVSSASTKGPSVFPLAPCSRSTSESTAALGCLVKDYFPEPVTVS
WNSGALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
52M51-H4 Heavy chain variable region amino acid sequence
(Putative signal sequence underlined)
SEQ ID NO: 13
MDWTWRVFCLLAVAPGVLSQVQLVQSGAEVKKPGASVKISCKVSGYTLRGYWIEWVRQAP
GKGLEWIGQILPGTGRTNYNEKFKGRVTMTADTSTDTAYMELSSLRSEDTAVYYCARFDG
NYGYYAMDYWGQGTTVTVSSA
52M51-H4 Heavy chain variable region amino acid sequence without
putative signal sequence
SEQ ID NO: 14
QVQLVQSGAEVKKPGASVKISCKVSGYTLRGYWIEWVRQAPGKGLEWIGQILPGTGRTNY
NEKFKGRVTMTADTSTDTAYMELSSLRSEDTAVYYCARFDGNYGYYAMDYWGQGTTVTVS
SA
52M51-L3 Light chain polynucleotide sequence (Putative signal
sequence is underlined)
SEQ ID NO: 15
ATGAGCGTCCCTACAATGGCTTGGATGATGCTCCTGCTGGGACTCCTGGCTTATGGAAGC
GGAGTGGATAGCCAGGCCGTCGTCACACAGGAACCTAGCCTCACCGTTAGCCCTGGAGGA
ACAGTCACACTGACCTGTAGGAGCTCCACAGGAGCTGTGACAACAAGCAATTACGCTAAC
TGGTTCCAGCAGAAGCCCGGTCAAGCCCCTAGAACCCTCATCGGCGGCACCAATAACAGA
GCTCCCGGAGTCCCCGCCAGGTTCTCCGGCTCCCTCCTGGGTGGCAAGGCTGCTCTGACA
CTCAGCGGTGCCCAGCCAGAGGATGAAGCGGAGTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
52M51-L3 Light chain amino acid sequence (Putative signal
sequence is underlined)
SEQ ID NO: 16
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
52M51-L3 Light chain variable region amino acid sequence
(Putative signal sequence is underlined)
SEQ ID NO: 17
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLG
52M51-L3 Light chain variable region amino acid sequence without
putative signal sequence
SEQ ID NO: 18
SGVDSQAVVTQEPSLTVSPGGTVTLTCRSSTGAVTTSNYANWFQQKPGQAPRTLIGGTNN
RAPGVPARFSGSLLGGKAALTLSGAQPEDEAEYYCALWYSNHWVFGGGTKLTVLG
52M51-L4 Light chain polynucleotide sequence (Putative signal
sequence is underlined)
SEQ ID NO: 19
ATGAGCGTCCCTACAATGGCTTGGATGATGCTCCTGCTGGGACTCCTGGCTTATGGAAGC
GGAGTGGATAGCCAGACCGTCGTCACACAGGAACCTAGCTTTTCCGTTAGCCCTGGAGGA
ACAGTCACACTGACCTGTAGGAGCTCCACAGGAGCTGTGACAACAAGCAATTACGCTAAC
TGGTATCAGCAGACTCCCGGTCAAGCCCCTAGAACCCTCATCGGCGGCACCAATAACAGA
GCTCCCGGAGTCCCCGACAGGTTCTCCGGCTCCATCCTGGGAAATAAAGCTGCTCTGACA
ATCACAGGTGCCCAGGCTGACGATGAAAGCGACTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
52M51-L4 Light chain amino acid sequence (Putative signal
sequence is underlined)
SEQ ID NO: 20
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
52M51-L4 Light chain variable region amino acid sequence
(Putative signal sequence is underlined)
SEQ ID NO: 21
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLG
52M51-L4 Light chain variable region amino acid sequence without
putative signal sequence
SEQ ID NO: 22
SGVDSQTVVTQEPSFSVSPGGTVTLTCRSSTGAVTTSNYANWYQQTPGQAPRTLIGGTNN
RAPGVPDRFSGSILGNKAALTITGAQADDESDYYCALWYSNHWVFGGGTKLTVLG
59R5 Heavy chain CDR1
SEQ ID NO: 23
SSSGMS
59R5 Heavy chain CDR2
SEQ ID NO: 24
VIASSGSNTYYADSVKG
59R5 Heavy chain CDR3
SEQ ID NO: 25
SIFYTT
59R5 Light chain CDR1
SEQ ID NO: 26
RASQSVRSNYLA
59R5 Light chain CDR2
SEQ ID NO: 27
GASSRAT
59R5 Light chain CDR3
SEQ ID NO: 28
QQYSNFPI
59R5 Heavy chain
SEQ ID NO: 29
EVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYTTWGQGTLVTVSSASTKG
PSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPK
PKDTLMISRTPEVTCVVVDVSHEDPEVQFNWYVDGVEVHNAKTKPREEQFNSTFRVVSVL
TVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCS
VMHEALHNHYTQKSLSLSPGK
59R5 Heavy chain variable region
SEQ ID NO: 30
EVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYTTWGQGTLVTVSSAST
Predicted protein sequence of anti-NOTCH2/3 59R5 light chain, plus
signal sequence. The signal sequence is underlined.
SEQ ID NO: 31
MVLQTQVFISLLLWISGAYGDIVLTQSPATLSLSPGERATLSCRASQSVRSNYLAWYQQKPGQAPRLL
IYGASSRATGVPARFSGSGSGTDFTLTISSLEPEDFAVYYCQQYSNFPITFGQGTKVEIKRTVAAPSV
FIFPPSDEQLKSGTASVVCLLNNFYPREAKVQWKVDNALQSGNSQESVTEQDSKDSTYSLSSTLTLSK
ADYEKHKVYACEVTHQGLSSPVTKSFNRGEC
59R1 Light chain VL of 59R5 IgG antibody
SEQ ID NO: 32
DIVLTQSPATLSLSPGERATLSCRASQSVRSNYLAWYQQKPGQAPRLLIYGASSRATGVPARFSGSGS
GTDFTLTISSLEPEDFAVYYCQQYSNFPITFGQGTKVEIKR
Minimal NOTCH1 receptor PEST domain
SEQ ID NO: 33
FLTPSPESP
Minimal NOTCH2/3 receptor PEST domain
SEQ ID NO: 34
YLTPSPESP
Minimal NOTCH4 receptor PEST domain
SEQ ID NO: 35
LTPSPE
NOTCH1 ICD peptide
SEQ ID NO: 36
VLLSRKRRRQHGQLW
NOTCH3 ICD peptide
SEQ ID NO: 37
VMVARRKREHSTLW

What is claimed is:

1. A method of identifying solid tumor cells that exhibit increased NOTCH receptor signaling, comprising identifying whether said cells contain a mutation in the proline, glutamate, serine, threonine-rich (PEST) domain of a human NOTCH receptor or in the TAD domain of human NOTCH1, wherein said solid tumor cells are selected from the group consisting of: a lung tumor, a glioma, a gastrointestinal tumor, a renal tumor, an ovarian tumor, a liver tumor, a colorectal tumor, an endometrial tumor, kidney tumor, prostate tumor, thyroid tumor, neuroblastoma, pancreatic tumor, glioblastoma multiforme, cervical tumor, stomach tumor, bladder tumor, hepatoma, breast tumor, colon tumor, melanoma, biliary tract tumor, and head and neck tumor.

2. A method for determining whether a patient diagnosed with cancer should be administered a NOTCH inhibitor, comprising:
(a) determining whether tumor cells from said patient contain an activating mutation of the PEST domain or the TAD domain of a human NOTCH receptor, wherein the presence of the mutation is predictive of said patient having a favorable response to treatment with a NOTCH inhibitor; and/or
(b) determining the level of NOTCH ICD in solid tumor cells from said patient, wherein a level of NOTCH ICD above a reference level is predictive of said patient having a favorable response to treatment with a NOTCH inhibitor.

3. The method of claim 1, wherein said NOTCH receptor is NOTCH1 or NOTCH3.

4. The method of claim 1, wherein said mutation is
a) a missense, nonsense, or frameshift mutation;
b) a frameshift or nonsense mutation of the PEST domain;
c) a deletion at nucleotide 7279 of the human NOTCH1 gene;
d) a guanine (G) deletion at nucleotide 7279 (B40 mutation) of the human NOTCH1 gene;
e) a missense mutation of the TAD domain;
f) a substitution at nucleotide 6733 of the human NOTCH1 gene;
g) a substitution to adenine (A) or cytosine (C) at nucleotide 6733 of the human NOTCH1 gene;
h) a substitution at nucleotide 6788 of the human NOTCH1 gene;
i) a substitution to adenine (A) at nucleotide 6788 of the human NOTCH1 gene;
j) an insertion at position 6622 of the human NOTCH3 gene;
k) a cytosine (C) insertion at position 6622 of the human NOTCH3 gene (B37 mutation);
l) an insertion at position 6096 of the human NOTCH3 gene; and/or
m) a cytosine (C) insertion at position 6096 of the human NOTCH3 gene.

5-6. (canceled)

7. The method of claim 2, further comprising administering a NOTCH inhibitor to said patient.

8. A method of treating cancer in a patient having a solid tumor comprising administering to said patient a therapeutically effective amount of a NOTCH inhibitor, wherein:
(a) at least one of the solid tumor cells in said patient comprises an activating mutation in the human NOTCH1 or NOTCH3 gene; and/or
(b) the solid tumor cells in said patient comprise NOTCH ICD at a level above a reference level.

9-10. (canceled)

11. The method of claim 8, wherein said mutation increases NOTCH signaling.

12. The method of claim 8, wherein at least about 0.1%, at least about 1%, at least about 2%, or at least about 5%, of the tumor cells from said patient comprise said mutation.

13. The method of claim 8, wherein said activating mutation of the human NOTCH1 receptor
a) is of the PEST domain;
b) is of the TAD domain;
c) increases NOTCH signaling;
d) comprises a guanine deletion at nucleotide 7279 of the human NOTCH1 gene;
e) comprises a substitution to adenine (A) or cytosine (C) at nucleotide 6733 of the human NOTCH1 gene; and/or
f) comprises a substitution to adenine (A) at nucleotide 6788 of the human NOTCH1 gene, or
wherein said activating mutation of the human NOTCH3 receptor
g) is of the PEST domain;
h) increases NOTCH signaling;
i) comprises a cytosine insertion at position 6622 of human NOTCH3 gene; and/or
j) comprises a cytosine insertion at position 6096 of human NOTCH3 gene.

14-15. (canceled)

16. The method of claim 2, further comprising administering a NOTCH inhibitor to said patient.

17-20. (canceled)

21. The method of claim 8, wherein the level of NOTCH ICD is the level in the nucleus of tumor cells.

22-25. (canceled)

26. The method of claim 8, wherein the solid tumor cells in said patient are characterized by an H-score of about 30 or more in an immunohistochemical assay with an anti-NOTCH1 ICD antibody.

27. The method of claim 8, wherein said NOTCH inhibitor is a gamma-secretase inhibitor or an anti-NOTCH antibody.

28. (canceled)

29. The method of claim 27, wherein said anti-NOTCH antibody is an anti-NOTCH1 antibody or an anti-NOTCH3 antibody.

30. The method of claim 29, wherein said anti-NOTCH1 antibody
a) blocks ligand binding to the NOTCH1 receptor;
b) blocks cleavage of the NOTCH1 receptor;
c) comprises a heavy chain variable region comprising CDR amino acid sequences CDR1 (SEQ ID NO:5), CDR2 (SEQ ID NO:6), and CDR3 (SEQ ID NO:7), and a light chain variable region comprising CDR amino acid sequences CDR1 (SEQ ID NO: 8); CDR2 (SEQ ID NO 9); and CDR3 (SEQ ID NO: 10);
d) comprises the heavy chain variable region sequence of SEQ ID NO:14 and the light chain variable region sequence of SEQ ID NO: 18; and/or
e) is OMP-52M51

31. The method of claim 29, wherein said anti-NOTCH3 antibody
a) blocks ligand biding to the NOTCH3 receptor;
b) blocks cleavage of the NOTCH3 receptor; and/or
c) comprises a heavy chain variable region comprising CDR amino acid sequences CDR1 (SEQ ID NO:23), CDR2 (SEQ ID NO:24), and CDR3 (SEQ ID NO:25), and a light chain variable region comprising CDR amino acid sequences CDR1 (SEQ ID NO: 26); CDR2 (SEQ ID NO: 27); and CDR3 (SEQ ID NO: 28).

32. The method of claim 8, wherein the method further comprises administering a second therapeutic agent.

33. The method of claim 8, wherein said patient is a human.

34. The method of claim 8, wherein said patient
a) comprises triple negative breast cancer cells;
b) previously failed a cancer therapy; and/or
c) has chemotherapy resistant breast cancer.

35. An isolated polynucleotide encoding a mutant human NOTCH1 or NOTCH3 receptor, wherein said polynucleotide comprises
a) a deletion at nucleotide 7279 of the human NOTCH1 gene;
b) a guanine (G) deletion at nucleotide 7279 of the human NOTCH1 gene;
c) an insertion at position 6622 of the human NOTCH3 gene;
d) a cytosine (C) insertion at position 6622 of the human NOTCH3 gene;
e) an insertion at position 6096 of the human NOTCH3 gene;
f) a cytosine (C) insertion at position 6096 of the human NOTCH3 gene;
g) a substitution at position 6733 of the human NOTCH1 gene;
h) an adenine (A) or cytosine (C) at position 6733 of the human NOTCH1 gene;
i) a substitution at position 6788 of the human NOTCH1 gene; and/or
j) an adenine (A) at position 6788 of the human NOTCH1 gene.

36. An isolated polypeptide encoded by the polynucleotide of claim 35.

37. A vector comprising the polynucleotide of claim 35.

38. A host cell transformed with the vector of claim 37.

39. (canceled)

40. A kit comprising at least one reagent for specifically detecting a mutated NOTCH receptor encoded for by the polvnucleotide of claim 35, wherein said reagent is optionally an antibody or nucleic acid probe that binds a mutated NOTCH receptor of the invention.


SEQUENCE LISTING (37 sequences)

SEQ ID NO: 1 - DNA, 9309 bp, Homo sapiens
  misc_feature (1)..(9309): Human NOTCH1 gene (wild-type)
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaaacggcgcgccccacgagaccccggcttcctttcccaagccttcgggcgtctgtgtgcgctctgtggatgccagggccgaccagaggagcctttttaaaacacatgtttttatacaaaataagaacgaggattttaattttttttagtatttatttatgtacttttattttacacagaaacactgcctttttatttatatgtactgttttatctggccccaggtagaaacttttatctattctgagaaaacaagcaagttctgagagccagggttttcctacgtaggatgaaaagattcttctgtgtttataaaatataaacaaagattcatgatttataaatgccatttatttattgattccttttttcaaaatccaaaaagaaatgatgttggagaagggaagttgaacgagcatagtccaaaaagctcctggggcgtccaggccgcgccctttccccgacgcccacccaaccccaagccagcccggccgctccaccagcatcacctgcctgttaggagaagctgcatccagaggcaaacggaggcaaagctggctcaccttccgcacgcggattaatttgcatctgaaataggaaacaagtgaaagcatatgggttagatgttgccatgtgttttagatggtttcttgcaagcatgcttgtgaaaatgtgttctcggagtgtgtatgccaagagtgcacccatggtaccaatcatgaatctttgtttcaggttcagtattatgtagttgttcgttggttatacaagttcttggtccctccagaaccaccccggccccctgcccgttcttgaaatgtaggcatcatgcatgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctgtaaatatgtttttaaaggtggattttgtttaaaaaatcttaatgaatgagtctgttgtgtgtcatgccagtgagggacgtcagacttggctcagctcggggagccttagccgcccatgcactggggacgctccgctgccgtgccgcctgcactcctcagggcagcctcccccggctctacgggggccgcgtggtgccatccccagggggcatgaccagatgcgtcccaagatgttgatttttactgtgttttataaaatagagtgtagtttacagaaaaagactttaaaagtgatctacatgaggaactgtagatgatgtatttttttcatcttttttgttaactgatttgcaataaaaatgatactgatggtgaaaaaaaaaaaaaaa

SEQ ID NO: 2 - DNA, 11189 bp, Homo sapiens
  misc_feature (1)..(11189): Human NOTCH2 gene (wild-type)
gcgaccgagaagatgcccgccctgcgccccgctctgctgtgggcgctgctggcgctctggctgtgctgcgcgacccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcgacatctcatccatgctttgtgtctcgaccctgcctgaatggcggcacatgccatatgctcagccgggatacctatgagtgcacctgtcaagtcgggtttacaggtaaggagtgccaatggaccgatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccttcagggcttcacaggccagtactgtgacagcctgtatgtgccctgtgcaccctcgccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcacatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaaaaaaaaaa

SEQ ID NO: 3 - DNA, 8089 bp, Homo sapiens
  misc_feature (1)..(8089): Human NOTCH3 gene (wild-type)
gcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcaccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggcgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgcgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccaggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccagaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcgggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggcggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttatttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagatgggacatgttccatagccttgctggggaagggcccactgccaactccctctgccccagccccacccttggccatctccctttgggaactagggggctgctggtgggaaatgggagccagggcagatgtatgcattcctttgtgtccctgtaaatgtgggactacaagaagaggagctgcctgagtggtactttctcttcctggtaatcctctggcccagcctcatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 4 - DNA, 6762 bp, Homo sapiens
  misc_feature (1)..(6762): Human NOTCH4 gene (wild-type)
agacgtgaggcttgcagcaggccgaggaggaagaagaggggcagtgggagcagaggaggtggctcctgccccagtgagagctctgagggtccctgcctgaagagggacagggaccggggcttggagaaggggctgtggaatgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatagaagaatacatggtagggaggaattccaaaaatgattacccattaaaaggcaggctggaaggccttcctggttttaagatggatcccccaaaatgaagggttgtgagtttagtttctctcctaaaatgaatgtatgcccaccagagcagacatcttccacgtggagaagctgcagctctggaaagagggtttaagatgctaggatgaggcaggcccagtcctcctccagaaaataagacaggccacaggagggcagagtggagtggaaatacccctaagttggaaccaagaattgcaggcatatgggatgtaagatgttctttcctatatatggtttccaaagggtgcccctatgatccattgtccccactgcccacaaatggctgacaaatatttattgggcacctactatgtgccaggcactgtgtaggtgctgaaaagtggccaagggccacccccgctgatgactccttgcattccctcccctcacaacaaagaactccactgtggggatgaagcgcttcttctagccactgctatcgctatttaagaaccctaaatctgtcacccataataaagctgatttgaagtgttaaaaaaaaaaaaaaaaaa

SEQ ID NO: 5 - protein, 6 aa, Artificial sequence
  52M51 Heavy chain CDR1
RGYWIE

SEQ ID NO: 6 - protein, 17 aa, Artificial sequence
  52M51 Heavy chain CDR2
QILPGTGRTNYNEKFKG

SEQ ID NO: 7 - protein, 12 aa, Artificial sequence
  52M51 Heavy chain CDR3
FDGNYGYYAMDY

SEQ ID NO: 8 - protein, 14 aa, Artificial sequence
  52M51 Light chain CDR1
RSSTGAVTTSNYAN

SEQ ID NO: 9 - protein, 7 aa, Artificial sequence
  52M51 Light chain CDR2
GTNNRAP

SEQ ID NO: 10 - protein, 16 aa, Artificial sequence
  52M51 Light chain CDR3
ALWYSNHWVFGGGTKL

SEQ ID NO: 11 - DNA, 1401 bp, Artificial Sequence
  52M51-H4 heavy chain polynucleotide sequence
atggattggacatggagggtgttctgcctcctcgctgtggctcctggagtcctgagccaggtccagctcgtccagagcggggctgaagtcaagaagcctggcgctagcgtcaaaatcagctgtaaggtcagcggatacacactgaggggatactggatcgagtgggtgaggcaggctccaggaaagggcctggaatggatcggccagatcctgcctggaaccggaaggacaaattacaatgagaagtttaagggaagggtcacaatgacagcagacacaagcacagacacagcttatatggaactcagctccctcagatccgaggacaccgctgtctactattgtgccaggttcgatggaaattacggatactatgccatggattactggggacaggggacaacggtcaccgtgagctcagccagcacaaagggccctagcgtcttccctctggctccctgcagcaggagcaccagcgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 466 aa, Artificial sequence
  52M51 H4 heavy chain amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 141 aa, Artificial sequence
  52M51-H4 heavy chain variable region amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 14 - protein, 122 aa, Artificial sequence
  52M51-H4 heavy chain variable region amino acid sequence without putative signal sequence
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 15 - DNA, 720 bp, Artificial Sequence
  52M51-L3 Light chain polynucleotide sequence
atgagcgtccctacaatggcttggatgatgctcctgctgggactcctggcttatggaagcggagtggatagccaggccgtcgtcacacaggaacctagcctcaccgttagccctggaggaacagtcacactgacctgtaggagctccacaggagctgtgacaacaagcaattacgctaactggttccagcagaagcccggtcaagcccctagaaccctcatcggcggcaccaataacagagctcccggagtccccgccaggttctccggctccctcctgggtggcaaggctgctctgacactcagcggtgcccagccagaggatgaagcggagtactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 16 - protein, 239 aa, Artificial sequence
  52M51-L3 light chain amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 17 - protein, 134 aa, Artificial sequence
  52M51-L3 light chain variable amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 18 - protein, 115 aa, Artificial sequence
  52M51-L3 light chain variable region amino acid sequence without putative signal sequence
SGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 19 - DNA, 720 bp, Artificial Sequence
  52M51-L4 Light chain polynucleotide sequence
atgagcgtccctacaatggcttggatgatgctcctgctgggactcctggcttatggaagcggagtggatagccagaccgtcgtcacacaggaacctagcttttccgttagccctggaggaacagtcacactgacctgtaggagctccacaggagctgtgacaacaagcaattacgctaactggtatcagcagactcccggtcaagcccctagaaccctcatcggcggcaccaataacagagctcccggagtccccgacaggttctccggctccatcctgggaaataaagctgctctgacaatcacaggtgcccaggctgacgatgaaagcgactactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 20 - protein, 239 aa, Artificial sequence
  52M51-L4 light chain amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 21 - protein, 134 aa, Artificial sequence
  52M51-L4 light chain variable region amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 22 - protein, 115 aa, Artificial sequence
  52M51-L4 light chain variable region amino acid sequence wtihout putative signal sequence
SGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 23 - protein, 6 aa, Artificial sequence
  59R5 heavy chain CDR1
SSSGMS

SEQ ID NO: 24 - protein, 17 aa, Artificial sequence
  59R5 heavy chain CDR2
VIASSGSNTYYADSVKG

SEQ ID NO: 25 - protein, 6 aa, Artificial sequence
  59R5 heavy chain CDR3
SIFYTT

SEQ ID NO: 26 - protein, 12 aa, Artificial sequence
  59R5 light chain CDR1
RASQSVRSNYLA

SEQ ID NO: 27 - protein, 7 aa, Artificial sequence
  59R5 light chain CDR2
GASSRAT

SEQ ID NO: 28 - protein, 8 aa, Artificial sequence
  59R5 light chain CDR3
QQYSNFPI

SEQ ID NO: 29 - protein, 441 aa, Artificial sequence
  59R5 heavy chain
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 118 aa, Artificial sequence
  59R5 heavy chain variable region
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSAST

SEQ ID NO: 31 - protein, 235 aa, Artificial sequence
  Predicted protein sequence of anti-NOTCH2/3 59R5 light chain, plus signal sequence
MVLQTQVFISLLLWISGAYGDIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 109 aa, Artificial sequence
  59R1 light chain VL of 59R5 IgG antibody
DIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKR

SEQ ID NO: 33 - protein, 9 aa, Artificial sequence
  Minimal NOTCH1 receptor PEST domain
FLTPSPESP

SEQ ID NO: 34 - protein, 9 aa, Artificial sequence
  Minimal NOTCH2/3 receptor PEST domain
YLTPSPESP

SEQ ID NO: 35 - protein, 6 aa, Artificial sequence
  Minimal NOTCH4 receptor PEST domain
LTPSPE

SEQ ID NO: 36 - protein, 15 aa, Artificial sequence
  NOTCH1 ICD peptide
VLLSRKRRRQHGQLW

SEQ ID NO: 37 - protein, 14 aa, Artificial sequence
  NOTCH3 ICD peptide
VMVARRKREHSTLW